(19) United States
(12) Patent Application Publication
(10) Pub. No.: US 2023/0142283 A1
(43) Pub. Date: May 11, 2023

(54) RIGID HELICAL JUNCTIONS FOR MODULAR REPEAT PROTEIN SCULPTING AND METHODS OF USE

(71) Applicant: University of Washington, Seattle, WA (US)

(72) Inventors: David BAKER, Seattle, WA (US); Matthew BICK, Seattle, WA (US); TJ BRUNETTE, Seattle, WA (US)

(21) Appl. No.: 17/759,175

(22) PCT Filed: Mar. 3, 2021
(86) PCT No.: PCT/US2021/020630

§ 371 Date: Jul. 20, 2022

Related U.S. Application Data
(60) Provisional application No. 62/985,760, filed on Mar. 5, 2020.

(51) Int. Cl. G16B 15/20; G16B 35/10; C12N 15/10; C07K 14/47; C07K 14/00

(52) U.S. Cl. CPC G16B 15/20; G16B 35/10; C12N 15/1068; C07K 14/47; C07K 14/001; C07K 2319/00

(57) ABSTRACT

Disclosed herein are junction polypeptides that can be used, for example, to join together protein building blocks via a rigid fusion to generate a wide range of protein shapes; fusion proteins comprising such junction polypeptides, polymers thereof, and methods for designing such junction polypeptides.

### CROSS REFERENCE

This application claims priority to U.S. Provisional Patent Application Ser. No. 62/985,760 filed Mar. 5, 2020, incorporated by reference herein in its entirety.

### FEDERAL FUNDING STATEMENT

This invention was made with government support under Grant Nos. OD018483 and P30 GM124169 and R01 GM118396 and R01 GM127648, awarded by the National Institutes of Health. The government has certain rights in the invention.

### Sequence Listing Statement

A computer readable form of the Sequence Listing is filed with this application by electronic submission and is incorporated into this application by reference in its entirety. The Sequence Listing is contained in the file created on Mar. 2, 2021, having the file name “21-0149-PCT Sequence-Listing ST25.txt” and is 345 kb in size.

### BACKGROUND

A modular combination of structured elements is difficult with proteins because they can adopt a wide variety of folds that are not universally complementary. The rigid body orientation of multiple protein domains with flexible linkers is not fixed, making it difficult to programmatically assemble larger structures using this approach. The design of complex structures would be considerably facilitated by general methods for rigidly fusing together pre-existing modules.

### SUMMARY

In one aspect, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NOS:1-78, wherein residues in parentheses are optional and may be present or absent. In one embodiment, the disclosure provides fusion proteins comprising the polypeptides of the first aspect. In another embodiment, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NOS:121-142, wherein residues in parentheses are optional and may be present or absent. In another embodiment, the disclosure provides polymers comprising 2 or more copies of the fusion proteins or polypeptides of preceding claim; in various embodiments the polymers comprise 3, 4, 5, 6, 7, 8, 9, 10, 15, 20, 25, 50, 75, 100, or more copies of the fusion protein or polypeptide.

The disclosure further provides libraries of the polypeptides, fusion proteins, and/or polymers of the disclosure; nucleic acids encoding the polypeptide or fusion protein of the disclosure, expression vectors comprising the nucleic acid of the disclosure operatively linked to a suitable control element; host cell comprising the polypeptide, fusion protein, polymer, nucleic acid, and/or expression vector of the disclosure, and methods for designing the polypeptides and fusion proteins of the disclosure.

### DESCRIPTION OF THE FIGURES

FIG. 1(a-e) A general method to create protein shapes using a library of designed junctions. a. Building blocks: (left, with different numbers of repeat units indicated in parentheses) Designed Helical Repeat proteins (DHRs), (middle) 20 homo-oligomers made from DHRs and (right) an ankyrin. b. Junctions can be made by superimposing helices. In this protocol, we overlap 6 residues in terminal repeats. The nearby residues are then redesigned (sticks). c. Junctions can also be made by building additional protein backbone as a contiguous chain with Rosetta™ fragment assembly. The DHRs are first trimmed of an entire helix and/or 1-4 terminal helix residues. Sequence near the interface is redesigned (sticks). d. Designs from both fusion methods are filtered to ensure they are lower in energy than other conformations in the energy landscape, contains two or more helices in contact throughout the junction, and there are no buried unsatisfied residues. (see examples, Discussion S2). e. The junction library is then used to sculpt proteins into various shapes. In this case, a repeat protein is connected first to a repeat protein followed by a repeat protein.

FIG. 2(a-b). Experimental characterization of the designed junction. a. Numbers of designs at each characterization stage; the overall success rate through the SAXS stage is 82%. b. Representative data for the four crystalized designs. Top row, junction names. Second row, the energy landscapes from Rosetta@Home simulations. The y-axis is energy as Rosetta™ Energy Units (roughly 1 kcal/mol) and the x-axis is the RMSD to the design. Third row, circular dichroism spectra collected at 25° C., 95° C. and then cooled to 25° C. All four proteins are stable to 95° C. The bottom row, crystal structures and RMSD.

FIG. 3. Characterization of long-armed junctions by negative stain EM. The designs shown match the EM averages at the resolution of the technique. Column 1: design model with each junction in a different shade of green or blue. Column 2: negative stain micrographs. Column 3: 2D class averages of the designs; the different views and orientations are consistent with the design models.

FIG. 4. Flowchart of the design protocol.

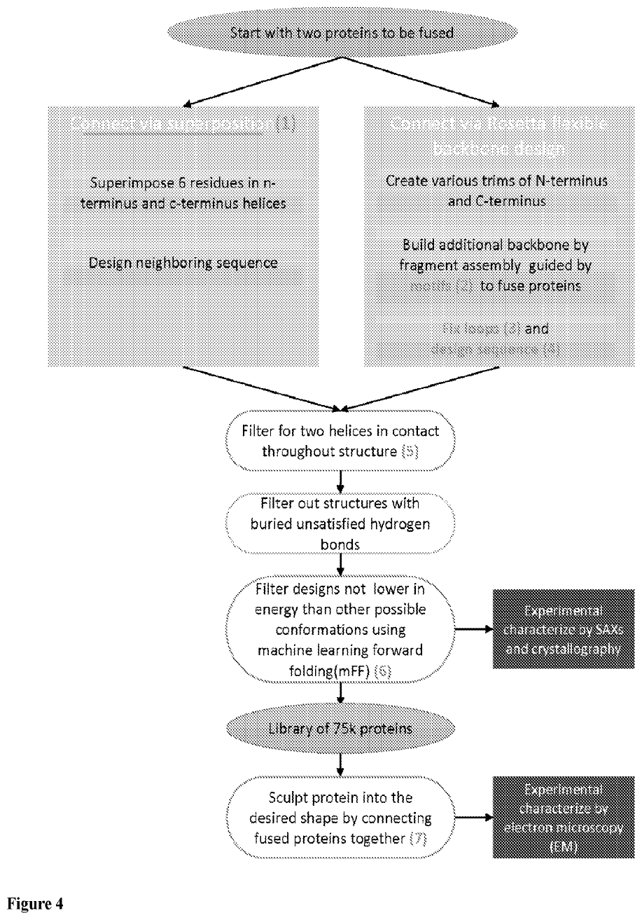

FIG. 5(a-e) a. Flowchart of the machine learning forward folding algorithm (mFF). 2250 Rosetta@Home simulations were used to train the model with 70% used for training and 30% set aside for testing. The Rosetta@Home simulations took two-three weeks to generate sufficient samples for training while each run of mFF took three-four hours on a single core. b, Exploration of the energy landscape by the different fragment sets in centroid. Fragment sets strongly biased toward the design focus exploration of the energy landscape on the region closest to the design, while weakly biased sets explore more broadly. c, to speed the algorithm, only a subset of centroid models are relaxed in the full-atom energy function. The decoys chosen for relaxing are the low energy cluster centers d, the roc curve, and e, the confusion matrix illustrates the accuracy of mFF as compared to the Rosetta@Home simulation.

FIG. 6(a-b). Fragment assembly sampling improvements. To evaluate Rosetta™ flexible backbone sampling improvements we designed approximately 2000 Denovo Helical Repeat (DHR) proteins with the sampling strategies described herein. Orig is the method from (8). RPX Motifs is a centroid score term that indicates when the backbone packs together with hydrophobic residues. Native loops replace fragment sampled loops with their closest natural loop. Structure profile biases the sequence design toward sequences of naturally occurring proteins. a. RPX motifs made a 116× improvement in sampling efficiency. Only 0.08% of designs made with the original method pass the centroid filtering while 9% pass with RPX motifs. b. After centroid sampling, full-atom design occurs. Designs are evaluated by what percent pass machine learning forward folding. We see a 1.6× improvement between the original Rosetta™ design procedure (motifs) and those designs generated with native loops and the structure profile.

FIG. 7. Structural validation by SAXS. Vr values for the fit of SAXs profiles to design models. The Vr cutoff value of 2.5 was calibrated using designs confirmed by crystallography. 28 of 30 designs were validated.

FIG. 8(a-b). Filtering of junction library. a. The number of designs left after each stage of filtering. Designs are filtered to 1.0 RMSD for uniqueness, 0 unsatisfied hydrogen bonds, 2 helices in connection throughout the structure, and lower energy than other conformation explored in the energy landscape (mFF). 52k designs pass all filters. Not all DHRs pass the filters so to enable all DHRs to be joined we also generated a second 75k database that includes junctions that were better than their component DHRs (See Discussion S5). b. The number of designs per junction correlates with the quality of the DHRs that make up the junction. Shown is the number of designs per DHR vs mFF quality of the component DHR. The counts in this graph are from the 75k library of junctions.

FIG. 9(a-c). Joinability of DHR. Illustrations of the DHRs that can be connected together after filtering a. via superposition of helices. b. via Rosetta™ fragment assembly and c. both methods

FIG. 10(a-c). Connections between junctions. a. From two junctions there are 4 possible structures, three of which are unique. The four ways to join junctions are by superimposing the outer two repeats (1), the inner two repeats (3) or one inner repeat to one outer repeat (2a and 2b). When one inner and one outer repeat is used the structure is identical independent of which of the junction provides the outer repeat. This is a byproduct of having 2 structurally identically and superimposable repeats at the end of each junction. Note: Sequence of connection type 1 are identical to the repeated sequence in the DHR. In case 2 and 3 each residue in the overlap derives its amino acid type for the residue from whichever building block has a residue closer. b. Superimposition of the 4 ways to join two junctions. The box highlights when the structure is identical. c. From the 75k designs in our databases, there are 542 million possible unique two junction combinations. If repeats protein extensions are counted the number of possibilities climbs into the billions.

FIG. 11. Ankyrin junction EM image. Characterization of DHR-ankyrin by negative stain EM. Column 1: design model. Column 2: raw negative stain micrograph. Column 3: 2D projections of the monomer design model. Column 4: 2D class averages of the design that appears to be structurally consistent with the 2D projection of monomer. Note the distinctive shape of the DHR component that is wider and shorter than the ankyrin component.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NOS:1-78, wherein residues in parentheses are optional and may be present or absent.

As disclosed in the examples that follow, the polypeptides of this first aspect are “junction” polypeptides that can be used, for example, to join together via a rigid fusion protein building blocks to generate a wide range of protein shapes. Such repeat proteins are excellent building blocks for protein-based nano-scale materials as they can readily be shortened or lengthened by changing the number of copies or repeats.

Sequences of exemplary polypeptides of the disclosure are provided in Table 1, wherein residues in parentheses are optional and may be present or absent.

TABLE 1

| Name | Sequence |
|---|---|
| Junction 1 | MDSEEVNERVKQLAEKAKEATDKEEVIEIVKELAELAKQSTDPEVVKEIVTQLAQVAQESTNEELIREIIEVLKELLKEAQ |
| DHR14-DHR14 | TPEEQAFIAAAIAAAAAKSGNEEEVRQAIQKAAELASQTSEESVKELVRELAELAKKAKDPKAVEAIVQLLAELAKKSSDS |
| | ELVNEIVKQLEEVAKEATDKELVEHIEKILEELKKQSTDGWLEHHHHHH(SEQ ID NO: 1) |
| | (M)DSEEVNERVKQLAEKAKEATDKEEVIEIVKELAELAKQSTDPEVVKEIVTQLAQVAQESTNEELIREIIEVLKELLKE |
| | AQTPEEQAFIAAAIAAAAAKSGNEEEVRQAIQKAAELASQTSEESVKELVRELAELAKKAKDPKAVEAIVQLLAELAKKSS |
| | DSELVNEIVKQLEEVAKEATDKELVEHIEKILEELKKQSTD(GWLEHHHHHH)(SEQ ID NO: 2) |
| Junction 2 | MDSEEVNERVKQLAEKAKEATDKEEVIEIVKELAELAKQSTDPELVLEILKQLIEVLKKSQNEELQEEILEVLKELLQLGD |
| DHR14-DHR54 | LEVILRAAQLAAKKGDQEVVRAALEAVAEKAIKAARKGNTDEVRKALEVALKIAEDAGTEEAVRLALEVVKRVSDEAKKQG |
| | NEDAVKEAEEVRKKIEEESGTGWLEHHHHHH(SEQ ID NO: 3) |
| | (M)DSEEVNERVKQLAEKAKEATDKEEVIEIVKELAELAKQSTDPELVLEILKQLIEVLKKSQNEELQEEILEVLKELLQL |
| | GDLEVILRAAQLAAKKGDQEVVRAALEAVAEKAIKAARKGNTDEVRKALEVALKIAEDAGTEEAVRLALEVVKRVSDEAKK |
| | QGNEDAVKEAEEVRKKIEEESGT(GWLEHHHHHH)(SEQ ID NO: 4) |
| Junction 3 | MDSEEVNERVKQLAEKAKEATDKEEVIEIVKELAELAKQSTDPNLVKEIVEQLLQVAQESTDEELLETILQVIKELAKNAQ |
| DHR14-DHR54 | SPEAALRAAEAILELAKEAGKLTEEEAKELLEIIARAAIEAARSGNVEAVRKALELALQVAKSAGTEEAVRLALEVVKRVS |
| | DEAKKQGNEDAVKEAEEVRKKIEEESGTGWLEHHHHHH(SEQ ID NO: 5) |
| | (M)DSEEVNERVKQLAEKAKEATDKEEVIEIVKELAELAKQSTDPNLVKEIVEQLLQVAQESTDEELLETILQVIKELAKN |
| | AQSPEAALRAAEAILELAKEAGKLTEEEAKELLEIIARAAIEAARSGNVEAVRKALELALQVAKSAGTEEAVRLALEVVKR |
| | VSDEAKKQGNEDAVKEAEEVRKKIEEESGT(GWLEHHHHHH)(SEQ ID NO: 6) |
| Junction 4 | MDSEEVNERVKQLAEKAKEATDKEEVIEIVKELAELAKQSTDPEVVAEIVTQLLQVAKESTDVELILEIAEVLLRLAEKAQ |
| DHR14-DHR71 | SKELASKALSSAVEAVTYLAELLKEGPPNPEAALEAAEAALQAARLAAENGNEEAFKKAAEAALQAAKILVEVASESGDPE |
| | LVEEAAKVAEEVRKLAKKQGDEEVYEKARETAREVKEELKRVREEKGDGWLEHHHHHH(SEQ ID NO: 7) |
| | (M)DSEEVNERVKQLAEKAKEATDKEEVIEIVKELAELAKQSTDPEVVAEIVTQLLQVAKESTDVELILEIAEVLLRLAEK |
| | AQSKELASKALSSAVEAVTYLAELLKEGPPNPEAALEAAEAALQAARLAAENGNEEAFKKAAEAALQAAKILVEVASESGD |
| | PELVEEAAKVAEEVRKLAKKQGDEEVYEKARETAREVKEELKRVREEKGD(GWLEHHHHHH)(SEQ ID NO: 8) |
| Junction 5 | MDSEEVNERVKQLAEKAKEATDKEEVIEIVKELAELAKQSTDPEVISEILELLEEVARKSTDKELILEIVQVILQLAKRNH |
| DHR14-DHR71 | GSPLAVKAARIAAKLAADAGDAELALRAAELAVEIARTAVENGDDEVAKEAAEAALEIAKKVVEAASEKGDPELVEEAAKV |
| | AEEVRKLAKKQGDEEVYEKARETAREVKEELKRVREEKGDGWLEHHHHHH(SEQ ID NO: 9) |
| | (M)DSEEVNERVKQLAEKAKEATDKEEVIEIVKELAELAKQSTDPEVISEILELLEEVARKSTDKELILEIVQVILQLAKR |
| | NHGSPLAVKAARIAAKLAADAGDAELALRAAELAVEIARTAVENGDDEVAKEAAEAALEIAKKVVEAASEKGDPELVEEAA |
| | KVAEEVRKLAKKQGDEEVYEKARETAREVKEELKRVREEKGD(GWLEHHHHHH)(SEQ ID NO: 10) |
| Junction 6 | MDSEEVNERVKQLAEKAKEATDKEEVIEIVKELAELAKQSTDPEVISEILELLEEVARKSTDKELILEIVQVILQLAKRNH |
| DHR14-DHR76 | GSPLAVKAARIAAKLAADAGDAELALRAAELAVEIARTAVENGDDEVAKEAAEAALEIAKKVVEAASEKGDPELVEEAAKV |
| | AEEVRKLAKKQGDEEVYEKARETAREVKEELKRVREEKGDGWLEHHHHHH(SEQ ID NO: 11) |
| | (M)DSEEVNERVKQLAEKAKEATDKEEVIEIVKELAELAKQSTDPEVISEILELLEEVARKSTDKELILEIVQVILQLAKR |
| | NHGSPLAVKAARIAAKLAADAGDAELALRAAELAVEIARTAVENGDDEVAKEAAEAALEIAKKVVEAASEKGDPELVEEAA |
| | KVAEEVRKLAKKQGDEEVYEKARETAREVKEELKRVREEKGD(GWLEHHHHHH)(SEQ ID NO: 12) |
| Junction 7 | MDSEEVNERVKQLAEKAKEATDKEEVIEIVKELAELAKQSTDPKVVAKILQALAEVAQQSTDPELARRIIEVIAELAKESG |
| DHR14-DHR79 | DEALLQAAEAAKEAAQKGNTELLLAVLQALLVAVEVLIVAEQARENGNEELAEAARELIRAVAEAITEAVQQGNPELVERV |
| | ARLAKKAAELIKRAIRAEKEGNRDERREALERVREVIERIEELVRQGNGWLEHHHHHH(SEQ ID NO: 13) |
| | (M)DSEEVNERVKQLAEKAKEATDKEEVIEIVKELAELAKQSTDPKVVAKILQALAEVAQQSTDPELARRIIEVIAELAKE |
| | SGDEALLQAAEAAKEAAQKGNTELLLAVLQALLVAVEVLIVAEQARENGNEELAEAARELIRAVAEAITEAVQQGNPELVE |
| | RVARLAKKAAELIKRAIRAEKEGNRDERREALERVREVIERIEELVRQGN(GWLEHHHHHH)(SEQ ID NO: 14) |
| Junction 8 | MDSEEVNERVKQLAEKAKEATDKEEVIEIVKELAELAKQSTDEKAIQEIAERLAEVAKESQDEELILTIILVLLNLLSTST |
| DHR14-DHR79 | DPEALEQIARAVLELARQNGDEELAQLAEEALRAVQTAKEAKEKGDEDLAQAALLIALAAAAAAAALIAAKQTGDPEVREL |
| | AQKLVELAQTAATQVKQNPKDEEVNEALKKIVKAIQEAVESLREAEESGDPEKREKARERVREAVERAEEVQRDPSSGWLE |
| | HHHHHH(SEQ ID NO: 15) |
| | (M)DSEEVNERVKQLAEKAKEATDKEEVIEIVKELAELAKQSTDEKAIQEIAERLAEVAKESQDEELILTIILVLLNLLST |
| | STDPEALEQIARAVLELARQNGDEELAQLAEEALRAVQTAKEAKEKGDEDLAQAALLIALAAAAAAAALIAAKQTGDPEVR |
| | ELAQKLVELAQTAATQVKQNPKDEEVNEALKKIVKAIQEAVESLREAEESGDPEKREKARERVREAVERAEEVQRDPSS(G |
| | WLEHHHHHH)(SEQ ID NO: 16) |
| Junction 9 | MDSEEVNERVKQLAEKAKEATDKEEVIEIVKELAELAKQSTDPTLISKIAERLTEVAEQGTNDELLVQIIYVLLRILQNGQ |
| DHR14-DHR79 | TDDLKKRVEKNAIKVLQKVVSNRDAADLAAKAVRKVAEDTLREHPDSSDVEKALKLVEEAQKAAERAREAADRTGTEDVQR |
| | LAQELIRLAIEAALQVVSDPSSEEVNEALKKIVKAIQEAVESLREAEESGDPEKREKARERVREAVERAEEVQRDPSSGWL |
| | EHHHHHH (SEQ ID NO:17) |
| | (M)DSEEVNERVRQLAERAREATDREEVIEIVRELAELARQSTDPTLISRIAERLTEVAEQGTNDELLVQIIYVLLRILQN |
| | GQTDDLRRRVERNAIRVLQKVVSNRDAADLAARAVRRVAEDTLREHPDSSDVERALRLVEEAQRAAERAREAADRTGTEDV |
| | QRLAQELIRLAIEAALQVVSDPSSEEVNEALKKIVKAIQEAVESLREAEESGDPEKREKARERVREAVERAEEVQRDPSS( |
| | GWLEHHHHHH)(SEQ ID NO: 18) |
| Junction 10 | MDSEEVNERVKQLAEKAKEATDKEEVIEIVKELAELAKQSTDPEVVLEIVEQLAQVATEAQDPELVSRILEVLARLAETLT |
| DHR14-DHR79 | NPEALSTVIQILTELARELLEQGNLEAAAEAIAIALEALARTTGDEEVRRAAELARLALQAAQEATEAAQRTGDPEVRRLA |
| | QRLARLAATAALQILQNPDDEEVNEALRRIVRAIQEAVESLREAEESGDPERRERARERVREAVERAEEVQRDPSSGWLEH |
| | HHHHH(SEQ ID NO: 19) |
| | (M)DSEEVNERVRQLAERAREATDREEVIEIVRELAELARQSTDPEVVLEIVEQLAQVATEAQDPELVSRILEVLARLAET |
| | LTNPEALSTVIQILTELARELLEQGNLEAAAEAIAIALEALARTTGDEEVRRAAELARLALQAAQEATEAAQRTGDPEVRR |
| | LAQRLARLAATAALQILQNPDDEEVNEALRRIVRAIQEAVESLREAEESGDPERRERARERVREAVERAEEVQRDPSS(GW |
| | LEHHHHHH)(SEQ ID NO: 20) |
| Junction 11 | MDSEEVNERVKQLAEKAKEATDKEEVIEIVKELAELAKQSTDTELVKKVVSLLAEVAVESKNEELIQEIIEVLKELISSIQ |
| DHR14-DHR79 | DPEQLRELAQELREQLQEALERGDYDAARVLAEALAAAARESGDEDLAEAARL1ARAAEA1RRAREAADRTGDPEVQRLAE |
| | ELARLALEAALQVLQDPRDEEVNEALRR1VRA1QEAVESLREAEESGDPERRERARERVREAVERAEEVQRDPSSGWLEHH |
| | HHHH(SEQ ID NO: 21) |
| | (M)DSEEVNERVRQLAERAREATDREEVIEIVRELAELARQSTDTELVKKVVSLLAEVAVESRNEELIQEIIEVLRELISS |
| | IQDPEQLRELAQELREQLQEALERGDYDAARVLAEALAAAARESGDEDLAEAARLIARAAEAIRRAREAADRTGDPEVQRL |
| | AEELARLALEAALQVLQDPRDEEVNEALRRIVRAIQEAVESLREAEESGDPERRERARERVREAVERAEEVQRDPSS(GWL |
| | EHHHHHH)(SEQ ID NO: 22) |
| Junction 12 | MDSEEVNERVKQLAEKAKEATDKEEVIEIVKELAELAKQSTDEEVIKRILELLKQVLKESTDPELQARILLVLARLASQQG |
| DHR14-DHR81 | NLREAARLAVRAAETAAKAGDQEALKEALEIARKALEEAQQQARQAKNEGDLETLAKALIAIALAIIAAAIVACTSGDKEE |
| | AERAYEDARRVEEEARKVKESAEEQGDSEVKRLAEEAEQLAREARRHVQECRGNGWLEHHHHHH(SEQ ID NO: 23) |
| | (M)DSEEVNERVKQLAEKAKEATDKEEVIEIVKELAELAKQSTDEEVIKRILELLKQVLKESTDPELQARILLVLARLASQ |
| | QGNLREAARLAVRAAETAAKAGDQEALKEALEIARKALEEAQQQARQAKNEGDLETLAKALIAIALAIIAAAIVACTSGDK |
| | EEAERAYEDARRVEEEARKVKESAEEQGDSEVKRLAEEAEQLAREARRHVQECRGN(GWLEHHHHHH)(SEQ ID |
| | NO: 24) |
| Junction 13 | MDSEEVNERVRQLAERAREATDREEVIEIVRELAELARQSTDPTLVARILADLAEAALEARDPELVQRIIEILQELARQAT |
| DHR14-DHR8 | SEDLLTIAQLAISAARAAQNGDEAVARVALALLQAVRLALENGNPEVAATIARVARRILEALRENPSDEMARRMLELARRV |
| | LDAARNNDDETAREIARQAAEEVEADRENNSGWLEHHHHHH(SEQ ID NO: 25) |
| | (M)DSEEVNERVRQLAERAREATDREEVIEIVRELAELARQSTDPTLVARILADLAEAALEARDPELVQRIIEILQELARQ |
| | ATSEDLLTIAQLAISAARAAQNGDEAVARVALALLQAVRLALENGNPEVAATIARVARRILEALRENPSDEMARRMLELAR |
| | RVLDAARNNDDETAREIARQAAEEVEADRENNS(GWLEHHHHHH)(SEQ ID NO: 26) |
| Junction 14 | MDSEEVNERVRQLAERAREATDREEVIEIVRELAELARQSTDPEAVREVAIQLAAVAAQAQDPELVRRIAQILEEILQQFP |
| DHR14-DHR8 | DDEAAREALQIARAILIVLEALHSSNSEEFRRVARALLEAVLLALENGDPRVALEIARAAEAIIRALRENPSDEMARRMLE |
| | LARRVLDAARNNDDETAREIARQAAEEVEADRENNSGWLEHHHHHH(SEQ ID NO: 27) |
| | (M)DSEEVNERVRQLAERAREATDREEVIEIVRELAELARQSTDPEAVREVAIQLAAVAAQAQDPELVRRIAQILEEILQQ |
| | FPDDEAAREALQIARAILIVLEALHSSNSEEFRRVARALLEAVLLALENGDPRVALEIARAAEAIIRALRENPSDEMARRM |
| | LELARRVLDAARNNDDETAREIARQAAEEVEADRENNS(GWLEHHHHHH)(SEQ ID NO: 28) |
| Junction 15 | MDIEKLCKKAESEAREARSKAEELRQRHPDSQAARDAQKLASQAEEAVKLACELAQEHPNADRAKACILLASAAAYAASKA |
| DHR18-DHR14 | VEDAQRHPDNQTARDKIKEASRIAELVIQFCRAAQENNDQKALDVLEKLATVASESGNEHVLKIIVEVLAILAQTITNKDD |
| | VIQAVDIARKIAEESTNSELVNEIVKQLEEVAKEATDKELVEHIEKILEELKKQSTDGWLEHHHHHH(SEQ ID |
| | NO: 29) |
| | (M)DIEKLCKKAESEAREARSKAEELRQRHPDSQAARDAQKLASQAEEAVKLACELAQEHPNADRAKACILLASAAAYAAS |
| | KAVEDAQRHPDNQTARDKIKEASRIAELVIQFCRAAQENNDQKALDVLEKLATVASESGNEHVLKIIVEVLAILAQTITNK |
| | DDVIQAVDIARKIAEESTNSELVNEIVKQLEEVAKEATDKELVEHIEKILEELKKQSTD(GWLEHHHHHH)(SEQ ID |
| | NO: 30) |
| Junction 16 | MDSEEEQERIRRILKEARKSGTEESLRQAIEDVAQLAKKSQDPEVIAHAVHVIAKIAQTSGSEEAKQQALRAVTEILSNAS |
| DHR49-DHR14 | EEEILEALKEALETAQQEGDDEALKLLVAAAAAAAKNSKDPDAIKEIVQLLLEAAKNSTDSELVNEIVKQLEEVAKEATDK |
| | ELVEHIEKILEELKKQSTDGWLEHHHHHH(SEQ ID NO: 31) |
| | (M)DSEEEQERIRRILKEARKSGTEESLRQAIEDVAQLAKKSQDPEVIAHAVHVIAKIAQTSGSEEAKQQALRAVTEILSN |
| | ASEEEILEALKEALETAQQEGDDEALKLLVAAAAAAAKNSKDPDAIKEIVQLLLEAAKNSTDSELVNEIVKQLEEVAKEAT |
| | DKELVEHIEKILEELKKQSTD(GWLEHHHHHH)(SEQ ID NO: 32) |
| Junction 17 | MDSEEEQERIRRILKEARKSGTEESLRQAIEDVAQLAKKSQDPEVLRTAVEVIKEIAETSGSPEALYEAIQAVIEIARSAQ |
| DHR49-DHR81 | DEEALATAAIAAAELADQLLQTASESGDEEALTEAAELAREILREARRVLEQAQRSGNLEVAAKALIAIALAILVIAKVAC |
| | QKGDKEEAERAYEDARRVEEEARKVKESAEEQGDSEVKRLAEEAEQLAREARRHVQECRGNGWLEHHHHHH(SEQ ID |
| | NO: 33) |
| | (M)DSEEEQERIRRILKEARKSGTEESLRQAIEDVAQLAKKSQDPEVLRTAVEVIKEIAETSGSPEALYEAIQAVIEIARS |
| | AQDEEALATAAIAAAELADQLLQTASESGDEEALTEAAELAREILREARRVLEQAQRSGNLEVAAKALIAIALAILVIAKV |
| | ACQKGDKEEAERAYEDARRVEEEARKVKESAEEQGDSEVKRLAEEAEQLAREARRHVQECRGN(GWLEHHHHHH)(SEQ |
| | ID NO: 34) |
| Junction 18 | MSNDEKEKLKELLKRAEELAKSPDPEDLKEAVRLAEEVVRERPGSEDAKKALKIVIKAAAELAKAPNPEALKEAIEALQKV |
| DHR54-DHR79 | AEHSNSEEVKEAIEAIKSVLEAAREALESGDEEAAQELARLAYRAAQLLIKLEDSQDDEEKKALLLAVQALAAAAQALQAA |
| | SQTGDPEVIELAQKLVELAETAATQVEQNPKDEEVNEALKKIVKAIQEAVESLREAEESGDPEKREKARERVREAVERAEE |
| | VQRDPSSGWLEHHHHHH(SEQ ID NO: 35) |
| | (M)SNDEKEKLKELLKRAEELAKSPDPEDLKEAVRLAEEVVRERPGSEDAKKALKIVIKAAAELAKAPNPEALKEAIEALQ |
| | KVAEHSNSEEVKEAIEAIKSVLEAAREALESGDEEAAQELARLAYRAAQLLIKLEDSQDDEEKKALLLAVQALAAAAQALQ |
| | AASQTGDPEVIELAQKLVELAETAATQVEQNPKDEEVNEALKKIVKAIQEAVESLREAEESGDPEKREKARERVREAVERA |
| | EEVQRDPSS(GWLEHHHHHH)(SEQ ID NO: 36) |
| Junction 19 | MTTEDERRELEKVARKAIEAAREGNTDEVREQLQRALEIARESGTKTAVKLALDVALRVAQEAAKRGNKDAIDEAAEVVVR |
| DHR54-DHR79 | IAEESNNSDALEQALRVLEEIAKAVLKSEKTEDAKKAVKLVQEAYKAAQRAIEAAKRTGTPDVIKLAIKLAKLAARAALEV |
| | IKRPKSEEVNEALKKIVKAIQEAVESLREAEESGDPEKREKARERVREAVERAEEVQRDPSSGWLEHHHHHH(SEQ ID |
| | NO: 37) |
| | (M)TTEDERRELEKVARKAIEAAREGNTDEVREQLQRALEIARESGTKTAVKLALDVALRVAQEAAKRGNKDAIDEAAEVV |
| | VRIAEESNNSDALEQALRVLEEIAKAVLKSEKTEDAKKAVKLVQEAYKAAQRAIEAAKRTGTPDVIKLAIKLAKLAARAAL |
| | EVIKRPKSEEVNEALKKIVKAIQEAVESLREAEESGDPEKREKARERVREAVERAEEVQRDPSS(GWLEHHHHHH)(SEQ |
| | ID NO: 38) |
| Junction 20 | MSSDEEEARELIERAKEAAERAQEAAERTGDPRVRELARELKRLAQEAAEEVKRDPSSRITLDILKAVIEAIEVAVRSLEK |
| DHR79-DHR14 | AYRNGNPEDVKKASKIVEEAVRLAEAATKGNYQEINKAAREATKNNNEDLVRIAVKAAAAAAKETQTKDDVKKIVDELRKI |
| | AKNNTNSELVNEIVKQLEEVAKEATDKELVEHIEKILEELKKQSTDGWLEHHHHHH(SEQ ID NO: 39) |
| | (M)SSDEEEARELIERAKEAAERAQEAAERTGDPRVRELARELKRLAQEAAEEVKRDPSSRITLDILKAVIEAIEVAVRSL |
| | EKAYRNGNPEDVKKASKIVEEAVRLAEAATKGNYQEINKAAREATKNNNEDLVRIAVKAAAAAAKETQTKDDVKKIVDELR |
| | KIAKNNTNSELVNEIVKQLEEVAKEATDKELVEHIEKILEELKKQSTD(GWLEHHHHHH)(SEQ ID NO: 40) |
| Junction 21 | MSSDEEEARELIERAKEAAERAQEAAERTGDPRVRELARELKRLAQEAAEEVKRDPSSKTTLIALKLIIIAIELAVRALEE |
| DHR79-DHR14 | AIKKGNPEEVKKATKIVEKAVRLAEEIQHGNQKQIARAAADIAKLAIESGNEDVARKVVKVVAELAQTGTNKDVVTEIVKA |
| | LEKIARQGTNSELVNEIVKQLEEVAKEATDKELVEHIEKILEELKKQSTDGWLEHHHHHH(SEQ ID NO: 41) |
| | (M)SSDEEEARELIERAKEAAERAQEAAERTGDPRVRELARELKRLAQEAAEEVKRDPSSKTTLIALKLIIIAIELAVRAL |
| | EEAIKKGNPEEVKKATKIVEKAVRLAEEIQHGNQKQIARAAADIAKLAIESGNEDVARKVVKVVAELAQTGTNKDVVTEIV |
| | KALEKIARQGTNSELVNEIVKQLEEVAKEATDKELVEHIEKILEELKKQSTD(GWLEHHHHHH)(SEQ ID NO: 42) |
| Junction 22 | MSSDEEEARELIERAKEAAERAQEAAERTGDPRVRELARELKRLAQEAAEEVKRDPSSKDTLRALSIIIIAIEVAVIALEV |
| DHR79-DHR54 | AQKQGNPKVKERASQLVEEAVRAAEEVQNDPTDDAVYNAVHTLARAALDAVKNGPDTRDVVKKALEVVARLAIIAARQGST |
| | DAVRDALKVALKIARTAGNEEAVRLALEVVKRVSDEAKKQGNEDAVKEAEEVRKKIEEESGTGWLEHHHHHH(SEQ ID |
| | NO: 43) |
| | (M)SSDEEEARELIERAKEAAERAQEAAERTGDPRVRELARELKRLAQEAAEEVKRDPSSKDTLRALSIIIIAIEVAVIAL |
| | EVAQKQGNPKVKERASQLVEEAVRAAEEVQNDPTDDAVYNAVHTLARAALDAVKNGPDTRDVVKKALEVVARLAIIAARQG |
| | STDAVRDALKVALKIARTAGNEEAVRLALEVVKRVSDEAKKQGNEDAVKEAEEVRKKIEEESGT(GWLEHHHHHH) |
| | (SEQ ID NO: 44) |
| Junction 23 | MDSEEVNERVKQLAEKAKEATDKEEVIEIVKELAELAKQSTDPNLVAEVVRALTEVAKTSTDTELIREIIKVLLELASKLR |
| DHR14-DHR18 | DPQAVLEALQAVAELARELAEKTGDPIAKECAEAVSAAAEAVKKAADLLKRHPGSEAAQAALELAKAAAEAVLIACLLALD |
| | YPKSDIAKKCIKAASEAAEEASKAAEEAQRHPDSQKARDEIKEASQKAEEVKERCERAQEHPNAGWLEHHHHHH(SEQ |
| | ID NO: 45) |
| | (M)DSEEVNERVKQLAEKAKEATDKEEVIEIVKELAELAKQSTDPNLVAEVVRALTEVAKTSTDTELIREIIKVLLELASK |
| | LRDPQAVLEALQAVAELARELAEKTGDPIAKECAEAVSAAAEAVKKAADLLKRHPGSEAAQAALELAKAAAEAVLIACLLA |
| | LDYPKSDIAKKCIKAASEAAEEASKAAEEAQRHPDSQKARDEIKEASQKAEEVKERCERAQEHPNA(GWLEHHHHHH) |
| | (SEQ ID NO: 46) |
| Junction 24 | MDSEEVNERVKQLAEKAKEATDKEEVIEIVKELAELAKQSTDPNVVAEIVYQLAEVAEHSTDPELIKEILQEALRLAEEQG |
| DHR14-DHR18 | DEELAEAARLALKAARLLEEARQLLSKDPENEAAKECLKAVRAALEAALLALLLLAKHPGSQAAQDAVQLATAALRAVEAA |
| | CQLAKQYPNSDIAKKCIKAASEAAEEASKAAEEAQRHPDSQKARDEIKEASQKAEEVKERCERAQEHPNAGWLEHHHHHH |
| | (SEQ ID NO: 47) |
| | (M)DSEEVNERVKQLAEKAKEATDKEEVIEIVKELAELAKQSTDPNVVAEIVYQLAEVAEHSTDPELIKEILQEALRLAEE |
| | QGDEELAEAARLALKAARLLEEARQLLSKDPENEAAKECLKAVRAALEAALLALLLLAKHPGSQAAQDAVQLATAALRAVE |
| | AACQLAKQYPNSDIAKKCIKAASEAAEEASKAAEEAQRHPDSQKARDEIKEASQKAEEVKERCERAQEHPNA(GWLEHHHH |
| | HH)(SEQ ID NO: 48) |
| Junction 25 | MDSEEVNERVKQLAEKAKEATDKEEVIEIVKELAELAKQSTDKEVVKRIVELLTEVAKESTDVELIAEIIAVLIELAAHAS |
| DHR14-DHR54 | SETLQEANQLIRELLHEAASGNKEAVQILLEAIAELAVKAARKGNVEAVKLALQAALEVAESAGTEEAVRLALEVVKRVSD |
| | EAKKQGNEDAVKEAEEVRKKIEEESGTGWLEHHHHHH(SEQ ID NO: 49) |
| | (M)DSEEVNERVKQLAEKAKEATDKEEVIEIVKELAELAKQSTDKEVVKRIVELLTEVAKESTDVELIAEIIAVLIELAAH |
| | ASSETLQEANQLIRELLHEAASGNKEAVQILLEAIAELAVKAARKGNVEAVKLALQAALEVAESAGTEEAVRLALEVVKRV |
| | SDEAKKQGNEDAVKEAEEVRKKIEEESGT(GWLEHHHHHH)(SEQ ID NO: 50) |
| Junction 26 | MDSEEVNERVKQLAEKAKEATDKEEVIEIVKELAELAKQSTDEELVNRIVEALEEVAKESTDPQLIIEILLVLALLAVESG |
| DHR14-DHR7I | GTEKADEALRRITEQAREAAQQGDAEAVLEAARAALQAAKAAAEKGDDEVFKSAAEAALTIAKELVEAASEKGDPELVEEA |
| | AKVAEEVRKLAKKQGDEEVYEKARETAREVKEELKRVREEKGDGWLEHHHHHH(SEQ ID NO: 51) |
| | (M)DSEEVNERVKQLAEKAKEATDKEEVIEIVKELAELAKQSTDEELVNRIVEALEEVAKESTDPQLIIEILLVLALLAVE |
| | SGGTEKADEALRRITEQAREAAQQGDAEAVLEAARAALQAAKAAAEKGDDEVFKSAAEAALTIAKELVEAASEKGDPELVE |
| | EAAKVAEEVRKLAKKQGDEEVYEKARETAREVKEELKRVREEKGD(GWLEHHHHHH)(SEQ ID NO: 52) |
| Junction 27 | MDSEEVNERVKQLAEKAKEATDKEEVIEIVKELAELAKQSTDPEVVKEIVEQLLQVAQEAQDPELVKEIIRILKELAKTAE |
| DHR14-DHR79 | NEEAAATALLAVAEALAVLAELLARTTGDDSARQAAELAKEAAEAAKRAQEAAKRTGDPEVKRLALELVRLAAEAAEEVTK |
| | NPDDEEVNEALKKIVKAIQEAVESLREAEESGDPEKREKARERVREAVERAEEVQRDPSSGWLEHHHHHH(SEQ ID |
| | NO: 53) |
| | (M)DSEEVNERVKQLAEKAKEATDKEEVIEIVKELAELAKQSTDPEVVKEIVEQLLQVAQEAQDPELVKEIIRILKELAKT |
| | AENEEAAATALLAVAEALAVLAELLARTTGDDSARQAAELAKEAAEAAKRAQEAAKRTGDPEVKRLALELVRLAAEAAEEV |
| | TKNPDDEEVNEALKKIVKAIQEAVESLREAEESGDPEKREKARERVREAVERAEEVQRDPSS(GWLEHHHHHH)(SEQ |
| | ID NO: 54) |
| Junction 28 | MDSEEVNERVKQLAEKAKEATDKEEVIEIVKELAELAKQSTDPTLVAKIAVLLAEVAAEAQDPELIKRILEILRQLIKNAK |
| DHR14-DHR79 | SDEARKAAKALAEAVEVALKAAQQLKQNPEDESARQALELILEAVEAAARALKAALETGSPEVIELALKLAELAIEAARQV |
| | LKNPDNEEVNEALKKIVKAIQEAVESLREAEESGDPEKREKARERVREAVERAEEVQRDPSSGWLEHHHHHH(SEQ ID |
| | NO: 55) |
| | (M)DSEEVNERVKQLAEKAKEATDKEEVIEIVKELAELAKQSTDPTLVAKIAVLLAEVAAEAQDPELIKRILEILRQLIKN |
| | AKSDEARKAAKALAEAVEVALKAAQQLKQNPEDESARQALELILEAVEAAARALKAALETGSPEVIELALKLAELAIEAAR |
| | QVLKNPDNEEVNEALKKIVKAIQEAVESLREAEESGDPEKREKARERVREAVERAEEVQRDPSS(GWLEHHHHHH)(SEQ |
| | ID NO: 56) |
| Junction 29 | MDSEEVNERVKQLAEKAKEATDKEEVIEIVKELAELAKQSTDKEAIKDIVRALKEVLKHSQDDELREQILIVLALLAAQAG |
| DHR14-DHR8 | DVEEALEALERLAQEAKEKGDEEALKVLKALAEAVRTAKENGNPEVAATVAEAAAKIATALRENPSDEMAKKMLELAKRVL |
| | DAAKNNDDETAREIARQAAEEVEADRENNSGWLEHHHHHH(SEQ ID NO: 57) |
| | (M)DSEEVNERVKQLAEKAKEATDKEEVIEIVKELAELAKQSTDKEAIKDIVRALKEVLKHSQDDELREQILIVLALLAAQ |
| | AGDVEEALEALERLAQEAKEKGDEEALKVLKALAEAVRTAKENGNPEVAATVAEAAAKIATALRENPSDEMAKKMLELAKR |
| | VLDAAKNNDDETAREIARQAAEEVEADRENNS(GWLEHHHHHH)(SEQ ID NO: 58) |
| Junction 30 | MDSEEVNERVKQLAEKAKEATDKEEVIEIVKELAELAKQSTDEEAVKEVVRQLALVAATATDPELIAEILQVILQLAEQAG |
| DHR14-DHR8 | DEEVAEAARQALEEIKQAQEQGSEAVALVLAALAVAVLAAAANGNPEVARVVKHAARLIKEALEENPSDEMAKKMLELAKR |
| | VLDAAKNNDDETAREIARQAAEEVEADRENNSGWLEHHHHHH(SEQ ID NO: 59) |
| | (M)DSEEVNERVKQLAEKAKEATDKEEVIEIVKELAELAKQSTDEEAVKEVVRQLALVAATATDPELIAEILQVILQLAEQ |
| | AGDEEVAEAARQALEEIKQAQEQGSEAVALVLAALAVAVLAAAANGNPEVARVVKHAARLIKEALEENPSDEMAKKMLELA |
| | KRVLDAAKNNDDETAREIARQAAEEVEADRENNS(GWLEHHHHHH)(SEQ ID NO: 60) |
| Junction 3I | MDSEEVNERVKQLAEKAKEATDKEEVIEIVKELAELAKQSTDKKLALQIVLLLAEVLQEAQDPELAIRIAEELAEIIKEAG |
| DHR14-DHR8 | GSEDALQIVQEIATALRQGNEEVAKVLAVLLIAVILALQNGNPEVAHEVARVAREILKALEENPTDEMAKKMLELAKRVLD |
| | AAKNNDDETAREIARQAAEEVEADRENNSGWLEHHHHHH(SEQ ID NO: 61) |
| | (M)DSEEVNERVKQLAEKAKEATDKEEVIEIVKELAELAKQSTDKKLALQIVLLLAEVLQEAQDPELAIRIAEELAEIIKE |
| | AGGSEDALQIVQEIATALRQGNEEVAKVLAVLLIAVILALQNGNPEVAHEVARVAREILKALEENPTDEMAKKMLELAKRV |
| | LDAAKNNDDETAREIARQAAEEVEADRENNS(GWLEHHHHHH)(SEQ ID NO: 62) |
| Junction 32 | MDSEEEQERIRRILKEARKSGTEESLRQAIEDVAQLAKKSQDEEVLREAVEVITQAARDSGSEEALQQAVRAVLEIAKSGK |
| DHR49-DHR79 | DVEAAAHAAKLLLEKNPEDESAREALELVERAVQAAQEAQEAANRTGDPEVQELAEKLLALAADAAAQVVKNPDDEEVNEA |
| | LKKIVKAIQEAVESLREAEESGDPEKREKARERVREAVERAEEVQRDPSSGWLEHHHHHH(SEQ ID NO: 63) |
| | (M)DSEEEQERIRRILKEARKSGTEESLRQAIEDVAQLAKKSQDEEVLREAVEVITQAARDSGSEEALQQAVRAVLEIAKS |
| | GKDVEAAAHAAKLLLEKNPEDESAREALELVERAVQAAQEAQEAANRTGDPEVQELAEKLLALAADAAAQVVKNPDDEEVN |
| | EALKKIVKAIQEAVESLREAEESGDPEKREKARERVREAVERAEEVQRDPSS(GWLEHHHHHH)(SEQ ID NO: 64) |
| Junction 33 | MSYEDECEEKARRVAEKVERLKRSGTSEDEIAEEVAREISEVIRTLKESGSSDEEIATCVALILAAAARALKESGVSDEQI |
| DHR4-DHR64 | NRILATLIKEVLRALNQETNKSNEEILRELLQALIELASKSDSETALLAVQLVVVLAKVALEVAQSEGSEEALELALEAAE |
| | EAARLAKEVLRLATENGNPEVARRAVELVKRVAELLERIARESGSEEAKERAERVREEARELQERVKELREREGDGWLEHH |
| | HHHH(SEQ ID NO: 65) |
| | (M)SYEDECEEKARRVAEKVERLKRSGTSEDEIAEEVAREISEVIRTLKESGSSDEEIATCVALILAAAARALKESGVSDE |
| | QINRILATLIKEVLRALNQETNKSNEEILRELLQALIELASKSDSETALLAVQLVVVLAKVALEVAQSEGSEEALELALEA |
| | AEEAARLAKEVLRLATENGNPEVARRAVELVKRVAELLERIARESGSEEAKERAERVREEARELQERVKELREREGD(GWL |
| | EHHHHHH)(SEQ ID NO: 66) |
| Junction 34 | MSNDEKEKLKELLKRAEELAKSPDPEDLKEAVRLAEEVVRERPGSEAAKKALEIIQEAAELLKKSPDPEAIIAAARALLKI |
| DHR53-DHR4 | AATTGDNEAAKQAIEAASKAAQLAEQRGDDELVCEALALLIAAQVLLLKQQGTSDEEVAEHVARTISQLVQRLKRKGASYE |
| | VIKECVQRIVEEIVEALKRSGTSEDEINEIVRRVKSEVERTLKESGSSGWLEHHHHHH(SEQ ID NO: 67) |
| | (M)SNDEKEKLKELLKRAEELAKSPDPEDLKEAVRLAEEVVRERPGSEAAKKALEIIQEAAELLKKSPDPEAIIAAARALL |
| | KIAATTGDNEAAKQAIEAASKAAQLAEQRGDDELVCEALALLIAAQVLLLKQQGTSDEEVAEHVARTISQLVQRLKRKGAS |
| | YEVIKECVQRIVEEIVEALKRSGTSEDEINEIVRRVKSEVERTLKESGSS(GWLEHHHHHH)(SEQ ID NO: 68) |
| ank_DHR18 | SVLGKVLIMAALVGNKDVVKVLIEVGADVNASLVSGATPLHAAAMNGHKEVVKLLISKGADVNALDEVGWTPLHLAVWVVL |
| | EIVECLLKNGADVNAADIDGYTPLHLAAFSGHLEIVEVLLKYGADVNADDQAGFTPLHLAAIFGHKEVVKLLISKGADLNT |
| | SAKDGATPVLLALRRGDEEVVRLLKEEAKKRGDEFLARCAEAAELAIEALKLAEELLRRYPNDEAARLAHHLAKLALEAVE |
| | LACILASEHPNADIAKLCIKAASEAAEAASKAAELAQRHPDSQAARDAIKLASQAAEAVKLACELAQEHPNADIAKKCIKA |
| | ASEAAEEASKAAEEAQRHPDSQKARDEIKEASQKAEEVKERCERAQE(SEQ ID NO: 69) |
| ank_DHR27 | SVLGKVLIMAALVGNKDVVKVLIEVGADVNASLVSGATPLHAAAMNGHKEVVKLLISKGADVNALDEVGWTPLHLAVWVVL |
| | EIVECLLKNGADVNAADIDGYTPLHLAAFSGHLEIVEVLLKYGADVNADDQAGFTPLHLAAIFGHKEVVKLLISKGADLNT |
| | SAKDGATPAALAASSGDKDVVETLERQARRNGDKELAQLAEVAREIYRLAEEARKLAKDEEEAKKIQKAANEAIAALALAV |
| | EKVTDNEVIEKLLEVVKEIIRLAEEAMKKMTDEEEAAKIAKEALEAIKMLARAVEEVTDKERIEQLLREVKEEIRRAEEES |
| | RKETDDEEAAKRAREALRRIRERAREVEE(SEQ ID NO: 70) |
| ank_DHR54 | SVLGKVLIMAALVGNKDVVKVLIEVGADVNASLVSGATPLHAAAMNGHKEVVKLLISKGADVNALDEVGWTPLHLAVWVVL |
| | EIVECLLKNGADVNAADIDGYTPLHLAAFSGHLEIVEVLLKYGADVNADDQAGFTPLHLAAIFGHKEVVKLLISKGADLNT |
| | SAKDGATPLLLAVRRGDEEAIRELLRELIERARESEEQAKRILHIILLAAEEAARRGNEEILRLALEAALEVARRSGTTEA |
| | VKLALEVVARVAIEAARRGNTDAVREALEVALEIARESGTEEAVRLALEVVKRVSDEAKKQGNEDAVKEAEEVRKKIEEES |
| | (SEQ ID NO: 71) |
| ank_DHR7 0 | SVLGKVLIMAALVGNKDVVKVLIEVGADVNASLVSGATPLHAAAMNGHKEVVKLLISKGADVNALDEVGWTPLHLAVWVVL |
| | EIVECLLKNGADVNAADIDGYTPLHLAAFSGHLEIVEVLLKYGADVNADDQAGFTPLHLAAIFGHKEVVKLLISKGADLNT |
| | SAKDGATPIALAIKRGDEEVAEKLIRSSSEEIIIEAARLAIEIARELLKKGDEELALRAARIALRAVRRLEEEARRTGSTE |
| | VLIEAARLAIEVARVALKVGSPETAREAVRTALELVQELERQARKTGSDEVLKRAAELAKEVARVAKEVGSPETARQARET |
| | AERLREELRRNREKK(SEQ ID NO: 72) |
| ank_DHR7I | SVLGKVLIMAALVGNKDVVKVLIEVGADVNASLVSGATPLHAAAMNGHKEVVKLLISKGADVNALDEVGWTPLHLAVWVVL |
| | EIVECLLKNGADVNAADIDGYTPLHLAAFSGHLEIVEVLLKYGADVNADDQAGFTPLHLAAIFGHKEVVKLLISKGADLNT |
| | SAKDGATPLLFAIKRGDEEAVRILLEELERRGEHNKEEALIAARIALKVAEIARRQGNEELFKEAAEIALRLAKLLVRIAK |
| | KEGDPELVLEAAKVALRVAELAAKNGDKEVFKKAAESALEVAKRLVEVASKEGDPELVEEAAKVAEEVRKLAKKQGDEEVY |
| | EKARETAREVKEELKRVREEK(SEQ ID NO: 73) |
| ank_DHR8 | SVLGKVLIMAALVGNKDVVKVLIEVGADVNASLVSGATPLHAAAMNGHKEVVKLLISKGADVNALDEVGWTPLHLAVWVVL |
| | EIVECLLKNGADVNAADIDGYTPLHLAAFSGHLEIVEVLLKYGADVNADDQAGFTPLHLAAIFGHKEVVKLLISKGADLNT |
| | SAKDGATPVILAARRGDEEVIELLLREAEKRGDEELLVIARLAQAIAIAKKNGNEEVAKEILKAALIIYEALRENNSDEMA |
| | KVMLALAKAVLLAAKNNDDEVAREIARAAAEIVEALRENNSDEMAKKMLELAKRVLDAAKNNDDETAREIARQAAEEVEAD |
| | RE(SEQ ID NO: 74) |
| DHR20_ank | SDIEEIRQLAEELRKKSDNEEVRKLAQEAAELAKRSTDSDVLEIVKDALELAKQSTNEEVIKLALKAAVLAAKSTDEEILK |
| | IVLEALRKARKSTNEEEILLILRAAVLAAKGDLEEALIIAARRGDEELVELARRGGADVNASLVSGATPLHAAAMNGHKEV |
| | VKLLISKGADVNALDEVGWTPLHLAVWVVLEIVECLLKNGADVNAADIDGYTPLHLAAFSGHLEIVEVLLKYGADVNADDQ |
| | AGFTPLHLAAIFGHKEVVKLLISKGADLNTSAKDGATPLDMARESGNEEVVKLLEKQ(SEQ ID NO: 75) |
| DHR21_ank | SEKEKVEELAQRIREQLPDTELAREAQELADEARKSDDSEALKVVYLALRIVQQLPDTELAREALELAKEAVKSTDEEILK |
| | AIYHALELVRRFPNTELAEAALLAALARQRGDEELAEKALILAAKRGSEEVVELARRAGADVNASLVSGATPLHAAAMNGH |
| | KEVVKLLISKGADVNALDEVGWTPLHLAVWVVLEIVECLLKNGADVNAADIDGYTPLHLAAFSGHLEIVEVLLKYGADVNA |
| | DDQAGFTPLHLAAIFGHKEVVKLLISKGADLNTSAKDGATPLDMARESGNEEVVKLLEKQ(SEQ ID NO: 76) |
| DHR55_ank | SVAEEIEKRAKKISKELKKEGKNPEWIEELQRAADKLVEVARRATSSDALEIAKRAVKIAEELAKQGSNPKWIAELLKAAA |
| | KLVEVAARATSEEALEIAKLAIKIAEELAKRGHDPEEIAEILKEAAKAVELARRGNLEEALIIAAKRGNEEIVEEARRGGA |
| | DVNASLVSGATPLHAAAMNGHKEVVKLLISKGADVNALDEVGWTPLHLAVWVVLEIVECLLKNGADVNAADIDGYTPLHLA |
| | AFSGHLEIVEVLLKYGADVNADDQAGFTPLHLAAIFGHKEVVKLLISKGADLNTSAKDGATPLDMARESGNEEVVKLLEKQ |
| | (SEQ ID NO: 77) |
| DHR18_ANK | SELGKRLIEAAENGNKDRVKDLIENGADVNASDSDGRTPLHHAAENGHKEVVKLLISKGADVNAKDSDGRTPLHHAAENGH |
| | KEVVKLLISKGADVNAKDSDGRTPLHHAAENGHKEVVKLLISKGADVNAKADRGMTPLHFAAWRGHKEVVKLLISKGADLN |
| | TSAKDGATPVLLALRRGDEEVVRLLKEEAKKRGDEFLARCAEAAELAIEALKLAEELLRRYPNDEAARLAHHLAKLALEAV |
| | ELACILASEHPNADIAKLCIKAASEAAEAASKAAELAQRHPDSQAARDAIKLASQAAEAVKLACELAQEHPNADIAKLCII |
| | AASLAAEAASKAAELAQRHPDSQAARDAIKLASQAAEAVKLACELAQEHPNAIIAILCIVAAIAAAIAASMAAALAQRHPD |
| | SQAARDAIKLASQAAEAVKLACELAQEHPNAKIAVLCILAAALAAIAAALAALLAQLHPDSQAARDAIKLASQAAEAVKLA |
| | CELAQEHPNADIAEKCILLAILAALLAILAALLAMLHPDSQLARDLIDLASELAEEVKERCER(SEQ ID NO: 78) |

In one embodiments, the polypeptide is at least 80%, 85%, 90%, 91%, 92%, 93%, 94%, 95%, 96%, 97%, 98%, 99%, or 100% identical to the amino acid sequence selected from SEQ ID NOS:1-78, wherein residues in parentheses are optional and may be present or absent. In another embodiment, the polypeptide is at least 90%, 91%, 92%, 93%, 94%, 95%, 96%, 97%, 98%, 99%, or 100% identical to the amino acid sequence selected from SEQ ID NOS:1-78, wherein residues in parentheses are optional and may be present or absent.

The polypeptides may include deletions of 1, 2, 3, 4, 5, 6, 7, 8, 9, or 10 amino acids relative to the N- or C-terminus of the polypeptide.

As noted above, the polypeptides of this first aspect are “junction” polypeptides that can be used, for example, to join together via a rigid fusion de novo designed repeat protein building blocks to generate a wide range of protein shapes. Thus, in another embodiment the disclosure provides fusion proteins comprising the polypeptides acting as junction polypeptides for repeat protein building blocks. In various non-limiting embodiments, the protein building blocks may comprise helix containing proteins, including but not limited to monomeric and homo-oligomeric de novo designed helix containing proteins (DHR) and ankyrin repeat proteins.

In non-limiting embodiments, the protein building blocks may comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO:79-120, and 143, wherein residues in parentheses (the N-terminal methionine residue) is optional and may be present or absent.

DHR3
(SEQ ID NO: 79)
(M)SSEDTVRKIAQKCSEAIRESNDCEEAARKCAKTISEAIRESNSSELAVRIIAQVCSEAIRESNDCECAARIC
AKIISEAIRESNSSELAVRIIAQVCSEAIRESNDCECAARICAKIISEAIRESNSSELAKRIIKQVCSEAKRESN
DTECAKRICTKIKSEAKRESNSWLE
DHR4
(SEQ ID NO: 80)
(M)SYEDECEEKARRVAEKVERLKRSGTSEDEIAEEVAREISEVIRTLKESGSSYEVICECVARIVAEIVEALKR
SGTSEDEIAEIVARVISEVIRTLKESGSSYEVICECVARIVAEIVEALKRSGTSEDEIAEIVARVISEVIRTLKE
SGSSYEVIKECVQRIVEEIVEALKRSGTSEDEINEIVRRVKSEVERTLKESGSS
DHR5
(SEQ ID NO: 81)
(M)SSEKEELRERLVKICVENAKRKGDDTEEAREAAREAFELVREAAERAGIDSSEVLELAIRLIKECVENAQRE
GYDISEACRAAAEAFKRVAEAAKRAGITSSEVLELAIRLIKECVENAQREGYDISEACRAAAEAFKRVAEAAKRA
GITSSETLKRAIEEIRKRVEEAQREGNDISEACRQAAEEFRKKAEELKRRGDG
DHR7
(SEQ ID NO: 82)
(M)STKEDARSTCEKAARKAAESNDEEVAKQAAKDCLEVAKQAGMPTKEAARSFCEAAARAAAESNDEEVAKIAA
KACLEVAKQAGMPTKEAARSFCEAAARAAAESNDEEVAKIAAKACLEVAKQAGMPTKEAARSFCEAAKRAAKESN
DEEVEKIAKKACKEVAKQAGMP
DHR8
(SEQ ID NO: 83)
(M)SDEMKKVMEALKKAVELAKKNNDDEVAREIERAAKEIVEALRENNSDEMAKVMLALAKAVLLAAKNNDDEVA
REIARAAAEIVEALRENNSDEMAKVMLALAKAVLLAAKNNDDEVAREIARAAAEIVEALRENNSDEMAKKMLELA
KRVLDAAKNNDDETAREIARQAAEEVEADRENNS
DHR9
(SEQ ID NO: 84)
(M)SYEDEAEEKARRVAEKVERLKRSGTSEDEIAEEVAREISEVIRTLKESGSSYEVIAEIVARIVAEIVEALKR
SGTSEDEIAEIVARVISEVIRTLKESGSSYEVIAEIVARIVAEIVEALKRSGTSEDEIAEIVARVISEVIRTLKE
SGSSYEVIKEIVQRIVEEIVEALKRSGTSEDEINEIVRRVKSEVERTLKESGSS
DHR10
(SEQ ID NO: 85)
(M)SSEKEELRERLVKIVVENAKRKGDDTEEAREAAREAFELVREAAERAGIDSSEVLELAIRLIKEVVENAQRE
GYDISEAARAAAEAFKRVAEAAKRAGITSSEVLELAIRLIKEVVENAQREGYDISEAARAAAEAFKRVAEAAKRA
GITSSETLKRAIEEIRKRVEEAQREGNDISEAARQAAEEFRKKAEELKRRGDG
DHR14
(SEQ ID NO: 86)
(M)DSEEVNERVKQLAEKAKEATDKEEVIEIVKELAELAKQSTDSELVNEIVKQLAEVAKEATDKELVIYIVKIL
AELAKQSTDSELVNEIVKQLAEVAKEATDKELVIYIVKILAELAKQSTDSELVNEIVKQLEEVAKEATDKELVEH
IEKILEELKKQSTDG
DHR15
(SEQ ID NO: 87)
(M)NDERQKQREEVRKLAEELASKATDEELIKEIKKCAQLAEELASRSTNDELIKQILEVAKLAFELASKATDEE
LIKEILKCCQLAFELASRSTNDELIKQILEVAKLAFELASKATDEELIKEILKCCQLAFELASRSTNDEEIKQIL
ETAKEAFERASKATDEEEIKEILKKCQEKFEKKSRSTNG
DHR18
(SEQ ID NO: 88)
(M)DIEKLCKKAESEAREARSKAEELRQRHPDSQAARDAQKLASQAEEAVKLACELAQEHPNADIAKLCIKAASE
AAEAASKAAELAQRHPDSQAARDAIKLASQAAEAVKLACELAQEHPNADIAKLCIKAASEAAEAASKAAELAQRH
PDSQAARDAIKLASQAAEAVKLACELAQEHPNADIAKKCIKAASEAAEEASKAAEEAQRHPDSQKARDEIKEASQ
KAEEVKERCERAQEHPNA
DHR20
(SEQ ID NO: 89)
(M)SDIEEIRQLAEELRKKSDNEEVRKLAQEAAELAKRSTDSDVLEIVKDALELAKQSTNEEVIKLALKAAVLAA
KSTDSDVLEIVKDALELAKQSTNEEVIKLALKAAVLAAKSTDEEVLEEVKEALRRAKESTDEEEIKEELRKAVEE
AESTDG
DHR21
(SEQ ID NO: 90)
(M)SEKEKVEELAQRIREQLPDTELAREAQELADEARKSDDSEALKVVYLALRIVQQLPDTELAREALELAKEAV
KSTDSEALKVVYLALRIVQQLPDTELAREALELAKEAVKSTDQEALKSVYEALQRVQDKPNTEEARESLERAKED
VKSTDG
DHR23
(SEQ ID NO: 91)
(M)SDSEKLAKRVLKELKRRGTSDEELERMKRELEKIIKSATSSDAMRLALRWLELVRRGTSSEILEKMMRMLI
KIIQSATSSDAMRLALRWLELVRRGTSSEILEKMMRMLIKIIQSATSDDQMREALRQVLEEVRKGTSSEQLERS
MRKLIKEIKKRTSG
DHR24
(SEQ ID NO: 92)
(M)SEAEELARRAAKEAKELCKRSTDEELCKELKKLAELLKELAERYPDSEAAKLALKAALEAIELCKQSTDEEL
CEELVKLAQKLIELAKRYPDSEAAKLALKAALEAIELCKQSTDEELCEELVKLAQKLIELAKRYPDSEEAKRALK
EAKELIEQCKESTDEDECRELVKRAEELIREAKENPDG
DHR26
(SEQ ID NO: 93)
(M)DECERLRQEVEKAEKELEKLAKQSTDEEVRQIAREVAKQLRRLAEEACRSNSDECLRLASEVVKAVQELVKL
AEQATDEEVIRVALEVARELIRLAQEACRSNDDECLRLASEVVKAVQELVKLAEQATDEEVIRVALEVARELIRL
AQEACRSNDEECLREASEVVKEVQELVKEAEKSTDEEEIRELLQRAEERIREAQERCREGDG
DHR27
(M)TRQKEQLDEVLEEIQRLAEEARKLMTDEEEAKKIQEEAERAKEMLRRAVEKVTDNEVIEKLLEVVKEIIRLA
EEAMKKMTDEEEAAKIAKEALEAIKMLARAVEEVTDNEVIEKLLEVVKEIIRLAEEAMKKMTDEEEAAKIAKEAL
EAIKMLARAVEEVTDKERIEQLLREVKEEIRRAEEESRKETDDEEAAKRAREALRRIRERAREVEEDKSG
(SEQ ID NO: 94)
DHR31
(SEQ ID NO: 95)
(M)DSYTERARKAVKRYVKEEGGSEEEAEREAEKVREEIRKKASDSYLIQAAAAVVAYVIEEGGSPEEAVKIAEE
VVRRIKEKADDSYLIQAAAAVVAYVIEEGGSPEEAVKIAEEVVRRIKEKADDRELIRRAAERVAEVIERGGSPEE
AVKEAEKEVKKQKEESDG
DHR32
(SEQ ID NO: 96)
(M)SIQEKAKQSVIRKVKEEGGSEEEARERAKEVEERLKKEADDSTLVRAAAAVVLYVLEKGGSTEEAVQRAREV
IERLKKEASDSTLVRAAAAVVLYVLEKGGSTEEAVQRAREVIERLKKEASDEELIREAAKEVLKVLEEGGSVEEA
VERARERIEELQKRSDDG
DHR36
(SEQ ID NO: 97)
(M)SDLEKALKRFVKEEKKKGRNPEEAKKEAKKLKKKLKKSAGSSDLLTALAKFVLEEVRKGRNPEEAVKEAIKL
AEKLKRSAGSSDLLTALAKFVLEEVRKGRNPEEAVKEAIKLAEKLKRSAGSSEQLEKLATKVLEEVKKGRNPKRA
VEEAIKQAKEDRKRSNSG
DHR39
(SEQ ID NO: 98)
(M)SDLQEVADRIVEQLKREGRSPEEARKEARRLIEEIKQSAGGDSELIEVAVRIVKELEEQGRSPSEAAKEAVE
LIERIRRAAGGDSELIEVAVRIVKELEEQGRSPSEAAKEAVELIERIRRAAGGDSDRIKKAVELVRELEERGRSP
SEAARRAVEEIQRSVEEDGGNG
DHR46
(SEQ ID NO: 99)
(M)STKEEKERIERIEKEVRSPDPENIREAVRKAEELLRENPSTEAEELLRRAIEAAVRAPDPEAIREAVRAAEE
LLRENPSTEAEELLRRAIEAAVRAPDPEAIREAVRAAEELLRENPSEEAKELLRRAIESAKKAPDPEAQREAKRA
EEELRKEDPG
DHR47
(SEQ ID NO: 100)
(M)STKEEKERIERIEKEVRSPDCENIREAVRKAEELLRENPSTEAEELLRRAIEAAVRCPDCEAIREAVRAAEE
LLRENPSTEAEELLRRAIEAAVRCPDCEAIREAVRAAEELLRENPSEEAKELLRRAIESAKKCPDPEAQREAKRA
EEELRKEDPG
DHR49
(SEQ ID NO: 101)
(M)DSEEEQERIRRILKEARKSGTEESLRQAIEDVAQLAKKSQDSEVLEEAIRVILRIAKESGSEEALRQAIRAV
AEIAKEAQDSEVLEEAIRVILRIAKESGSEEALRQAIRAVAEIAKEAQDPRVLEEAIRVIRQIAEESGSEEARRQ
AERAEEEIRRRAQG
DHR52
(SEQ ID NO: 102)
(M)QCEDRKEKIRELERKARENTGSDEARQAVKEIARIAKEALEEGCCDTAKEAIQRLEDLARDYSGSDVASLAV
KAIAKIAETALRNGCCDTAKEAIQRLEDLARDYSGSDVASLAVKAIAKIAETALRNGCKETAEEAIKRLRELAED
YKGSEVAKLAEEAIERIEKVSRERGG
DHR53
(SEQ ID NO: 103)
(M)SNDEKEKLKELLKRAEELAKSPDPEDLKEAVRLAEEVVRERPGSNLAKKALEIILRAAEELAKLPDPEALKE
AVKAAEKVVREQPGSNLAKKALEIILRAAEELAKLPDPEALKEAVKAAEKVVREQPGSELAKKALEIIERAAEEL
KKSPDPEAQKEAKKAEQKVREERPGG
DHR54
(SEQ ID NO: 104)
(M)TTEDERRELEKVARKAIEAAREGNTDEVREQLQRALEIARESGTTEAVKLALEVVARVAIEAARRGNTDAVR
EALEVALEIARESGTTEAVKLALEVVARVAIEAARRGNTDAVREALEVALEIARESGTEEAVRLALEVVKRVSDE
AKKQGNEDAVKEAEEVRKKIEEESGG
DHR55
(SEQ ID NO: 105)
(M)SSVAEEIEKRAKKISKELKKEGKNPEWIEELQRAADKLVEVARRATSSDALEIAKRAVKIAEELAKQGSNPK
WIAELLKAAAKLVEVAARATSSDALEIAKRAVKIAEELAKQGSNPKWIAELLKAAAKLVEVAARATSPKALKQAK
EAVKEAEELAKKGRNPKEIAEELKKRAKEVEKLARSTG
DHR57
(SEQ ID NO: 106)
(M)STEELKKVLERVRELSERAKESTDPEEALKIAKEVIELALKAVKEDPSTDALRAVLEAVRLASEVAKRVTDP
DKALKIAKLVIELALEAVKEDPSTDALRAVLEAVRLASEVAKRVTDPDKALKIAKLVIELALEAVKEDPSEEAKR
AVEEAKRLAEEVSKRVTDPELSEKIRQLVKELEEEAQKEDPG
DHR58
(SEQ ID NO: 107)
(M)STEELKKVLERVRELCERAKESTDPEEALKIAKEVIELALKAVKEDPSTDALRAVLEAVRCACEVAKRVTDP
DKALKIAKLVIELALEAVKEDPSTDALRAVLEAVRCACEVAKRVTDPDKALKIAKLVIELALEAVKEDPSEEAKR
AVEEAKRCAEEVSKRVTDPELSEKIRQLVKELEEEAQKEDPG
DHR59
(SEQ ID NO: 108)
(M)KTEVEKKAKEVIKEAKELAKELDSEEAKKVVERIKEAAEAAKRAAEQGKTEVAKLALKVLEEAIELAKENRS
EEALKVVLEIARAALAAAQAAEEGKTEVAKLALKVLEEAIELAKENRSEEALKVVLEIARAALAAAQAAEEGKSD
EARDALRRLEEAIEEAKENRSKESLEKVREEAKEAEQQAEDAREGG
DHR62
(SEQ ID NO: 109)
(M)DNDEKRKRAEKALQRAQEAEKKGDVEEAVRAAQEAVRAAKESGDNDVLRKVAEQALRIAKEAEKQGNVEVAV
KAARVAVEAAKQAGDNDVLRKVAEQALRIAKEAEKQGNVEVAVKAARVAVEAAKQAGDQDVLRKVSEQAERISKE
AKKQGNSEVSEEARKVADEAKKQTGG
DHR64
(SEQ ID NO: 110)
(M)DPEDELKRVEKLVKEAEELLRQAKEKGSEEDLEKALRTAEEAAREAKKVLEQAEKEGDPEVALRAVELVVRV
AELLLRIAKESGSEEALERALRVAEEAARLAKRVLELAEKQGDPEVALRAVELVVRVAELLLRIAKESGSEEALE
RALRVAEEAARLAKRVLELAEKQGDPEVARRAVELVKRVAELLERIARESGSEEAKERAERVREEARELQERVKE
LREREGG
DHR68
(SEQ ID NO: 111)
(M)TPRERLEEAKERVEEIRELIDKARKLQEQGNKEEAEKVLREAREQIREVTRELEEIAKNSDTPELALRAAEL
LVRLIKLLIEIAKLLQEQGNKEEAEKVLREATELIKRVTELLEKIAKNSDTPELALRAAELLVRLIKLLIEIAKL
LQEQGNKEEAEKVLREATELIKRVTELLEKIAKNSDTPELAKRAAELLKRLIELLKEIAKLLEEEGNEDEAEKVK
EEAKELEERVRELEERIRKNSDG
DHR70
(SEQ ID NO: 112)
(M)STEEKIEEARQSIKEAERSLREGNPEKAREDVRRALELVRELEKLARKTGSTEVLIEAARLAIEVARVALKV
GSPETAREAVRTALELVQELERQARKTGSTEVLIEAARLAIEVARVALKVGSPETAREAVRTALELVQELERQAR
KTGSDEVLKRAAELAKEVARVAKEVGSPETARQARETAERLREELRRNREKKGG
DHR71
(SEQ ID NO: 113)
(M)DPEEILERAKESLERAREASERGDEEEFRKAAEKALELAKRLVEQAKKEGDPELVLEAAKVALRVAELAAKN
GDKEVFKKAAESALEVAKRLVEVASKEGDPELVLEAAKVALRVAELAAKNGDKEVFKKAAESALEVAKRLVEVAS
KEGDPELVEEAAKVAEEVRKLAKKQGDEEVYEKARETAREVKEELKRVREEKGG
DHR72
(SEQ ID NO: 114)
(M)DSTKEKARQLAEEAKETAEKVGDPELIKLAEQASQEGDSEKAKAILLAAEAARVAKEVGDPELIKLALEAAR
RGDSEKAKAILLAAEAARVAKEVGDPELIKLALEAARRGDSEKARAILEAAERAREAKERGDPEQIKKARELAKR
GG
DHR76
(SEQ ID NO: 115)
(M)NPELEEVVIRRAKEVAKEVEKVAQRAEEEGNPDLRDSAKELRRAVEEAIEEAKKQGNPELVEWVARAAKVAAE
VIKVAIQAEKEGNRDLFRAALELVRAVIEAIEEAVKQGNPELVEWVARAAKVAAEVIKVAIQAEKEGNRDLFRAA
LELVRAVIEAIEEAVKQGNPELVERVARLAKKAAELIKRAIRAEKEGNRDERREALERVREVIERIEELVRQGG
DHR77
(SEQ ID NO: 116)
(M)NSDEEEAREWAERAEEAAKEALEQAKREGDEDARRVAEELEKQAEEARRKKDSEEAEAVYWAARAVLAALEA
LEQAKREGDEDARRVAEELLRQAEEAARKKNSEEAEAVYWAARAVLAALEALEQAKREGDEDARRVAEELLRQAE
EAARKKNPEEARAVYEAARDVLEALQRLEEAKRRGDEEERREAEERLRQAEERARKKG
DHR78
(SEQ ID NO: 117)
(M)NSDEEEAREWAERAEEAAKEALEQAKREGDEDARRCAEELEKQAEEARRKKDSEEAEAVYWAARAVLAALEA
LEQAKREGDEDARRCAEELLRQACEAARKKNSEEAEAVYWAARAVLAALEALEQAKREGDEDARRCAEELLRQAC
EAARKKNPEEARAVYEAARDVLEALQRLEEAKRRGDEEERREAEERLRQACERARKKG
DHR79
(SEQ ID NO: 118)
(M)SSDEEEARELIERAKEAAERAQEAAERTGDPRVRELARELKRLAQEAAEEVKRDPSSSDVNEALKLIVEAIE
AAVRALEAAERTGDPEVRELARELVRLAVEAAEEVQRNPSSSDVNEALKLIVEAIEAAVRALEAAERTGDPEVRE
LARELVRLAVEAAEEVQRNPSSEEVNEALKKIVKAIQEAVESLREAEESGDPEKREKARERVREAVERAEEVQRD
PSG
DHR80
(SEQ ID NO: 119)
(M)NSEELERESEEAERRLQEARKRSEEARERGDLKELAEALIEEARAVQELARVASERGNSEEAERASEKAQRV
LEEARKVSEEAREQGDDEVLALALIAIALAVLALAEVASSRGNSEEAERASEKAQRVLEEARKVSEEAREQGDDE
VLALALIAIALAVLALAEVASSRGNKEEAERAYEDARRVEEEARKVKESAEEQGDSEVKRLAEEAEQLAREARRH
VQETRGG
DHR81
(SEQ ID NO: 120)
(M)NSEELERESEEAERRLQEARKRSEEARERGDLKELAEALIEEARAVQELARVACERGNSEEAERASEKAQRV
LEEARKVSEEAREQGDDEVLALALIAIALAVLALAEVACCRGNSEEAERASEKAQRVLEEARKVSEEAREQGDDE
VLALALIAIALAVLALAEVACCRGNKEEAERAYEDARRVEEEARKVKESAEEQGDSEVKRLAEEAEQLAREARRH
VQECRGG
DHR82
(SEQ ID NO: 143)
(M)NDEEVQEAVERAEELREEAEELIKKARKTGDPELLRKALEALEEAVRAVEEAIKRNPDNDEAVETAVRLARE
LKKVAEELQERAKKTGDPELLKLALRALEVAVRAVELAIKSNPDNDEAVETAVRLARELKKVAEELQERAKKTGD
PELLKLALRALEVAVRAVELAIKSNPDNEEAVETAKRLAEELRKVAELLEERAKETGDPELQELAKRAKEVADRA
RELAKKSNPNG

In various specific embodiments, the fusion protein comprises the general formula X1-X2-X3, wherein the fusion protein is selected from the group consisting of, the following (taken from Table 1 examples; see the left-hand column) wherein residues in parentheses are optional and may be present or absent:

(a) X1 and X3 each independently is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86, wherein residues in parentheses are optional; and X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:1-2, wherein residues in parentheses are optional;

(b) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:3-4, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104;

(c) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:5-6, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104;

(d) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7-8, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:113;

(e) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:9-10, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:113;

(f) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:11-12, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:115;

(g) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction 80%, 85%, 90%, 91%, 92%, 93%, 94%, 95%, 96%, 97%, 98%, 99%, or 100% identical to the amino acid sequence of SEQ ID NO:13-14, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;

(h) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:15-16, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;

(i) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:17-18, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;

(j) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:19-20, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;

(k) X1 is a DHR polypeptide comprising an amino acid sequence at least 50%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:21-22, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;

(l) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:23-24, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:120;

(m) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:25-26, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:83;

(n) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:27-28, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:83;

(o) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:88; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:29-30, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86;

(p) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:101; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:31-32, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86;

(q) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:101; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:33-34, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:120;

(r) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:35-36, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;

(s) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:37-38, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;

(t) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:39-40, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86;

(u) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:41-42, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86;

(v) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:43-44, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104;

(w) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:45-46, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:88;

(x) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:47-48, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:88;

(y) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:49-50, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104;

(z) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction 80%, 85%, 90%, 91%, 92%, 93%, 94%, 95%, 96%, 97%, 98%, 99%, or 100% identical to the amino acid sequence of SEQ ID NO:51-52, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:113;

(aa) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:53-54, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;

(bb) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:55-56, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;

(cc) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:57-58, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:83;

(dd) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:59-60, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:83;

(ee) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:61-62, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:83;

(ff) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:101; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:63-64, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;

(gg) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:80; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:65-66, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:110; and

(hh) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:103; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:67-68, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:80.

The above embodiments are exemplified in the examples. In one specific embodiment, each of X1, X2, an X3 are at least 60% identical to the reference polypeptide. In another embodiment, each of X1, X2, an X3 are at least 75% identical to the reference polypeptide. In a further embodiment, each of X1, X2, an X3 are at least 80% identical to the reference polypeptide. In another embodiment, each of X1, X2, an X3 are at least 85% identical to the reference polypeptide. In one embodiment, each of X1, X2, an X3 are at least 90% identical to the reference polypeptide. In another embodiment, each of X1, X2, an X3 are at least 95% identical to the reference polypeptide. In a further embodiment, each of X1, X2, an X3 are 100% identical to the reference polypeptide.

The fusion proteins may include deletions of 1, 2, 3, 4, 5, 6, 7, 8, 9, or 10 amino acids relative to the N- or C-terminus of the polypeptide.

As described in detail in the examples that follow, the fusion proteins are combinatorial, and can be used to generate polymers.

In one embodiment, the fusion protein comprises the general formula X1-X2-X3-X4, wherein X4 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to a junction polypeptide that can be used to form a junction with X3 as shown in Table 1. The left-hand column of Table 1 provides exemplary junction polypeptides that can be used with exemplary DHR polypeptides. For example:

Junction 1 (SEQ ID NO:1-2) can be used to join two DHR14 polypeptides (SEQ ID NO: 86);

Junction 2 (SEQ ID NO:3-4) can be used to join DHR14 (SEQ ID NO: 86)-DHR54 (SEQ ID NO: 104);

Junction 3 (SEQ ID NO:5-6) can be used to join DHR14 (SEQ ID NO: 86)-DHR54 (SEQ ID NO: 104);

Junction 4 (SEQ ID NO:7-8) can be used to join DHR14 (SEQ ID NO: 86)-DHR71 (SEQ ID NO: 113);

Junction 5 (SEQ ID NO:9-10) can be used to join DHR14 (SEQ ID NO: 86)-DHR71 (SEQ ID NO: 113);

Junction 6 (SEQ ID NO:11-12) can be used to join DHR14 (SEQ ID NO: 86)-DHR76 (SEQ ID NO: 115);

Junction 7 (SEQ ID NO:13-14) can be used to join DHR14 (SEQ ID NO: 86)-DHR79 (SEQ ID NO: 118);

Junction 8 (SEQ ID NO:15-16) can be used to join DHR14 (SEQ ID NO: 86)-DHR79 (SEQ ID NO: 118);

Junction 9 (SEQ ID NO:17-18) can be used to join DHR14 (SEQ ID NO: 86)-DHR79 (SEQ ID NO: 118);

Junction 10 (SEQ ID NO:19-20) can be used to join DHR14 (SEQ ID NO: 86)-DHR79 (SEQ ID NO: 118);

Junction 11 (SEQ ID NO:21-22) can be used to join DHR14 (SEQ ID NO: 86)-DHR79 (SEQ ID NO: 118);

Junction 12 (SEQ ID NO:23-24) can be used to join DHR14 (SEQ ID NO: 86)-DHR81 (SEQ ID NO: 120);

Junction 13 (SEQ ID NO:25-26) can be used to join DHR14 (SEQ ID NO: 86)-DHR8 (SEQ ID NO: 83);

Junction 14 (SEQ ID NO:27-28) can be used to join DHR14 (SEQ ID NO: 86)-DHR8 (SEQ ID NO: 83);

Junction 15 (SEQ ID NO:29-30) can be used to join DHR18 (SEQ ID NO: 88)-DHR14 (SEQ ID NO: 86);

Junction 16 (SEQ ID NO:31-32) can be used to join DHR49 (SEQ ID NO: 101)-DHR14 (SEQ ID NO: 86);

Junction 17 (SEQ ID NO:33-34) can be used to join DHR49 (SEQ ID NO: 101)-DHR81 (SEQ ID NO: 120);

Junction 18 (SEQ ID NO:35-36) can be used to join DHR54 (SEQ ID NO: 104)-DHR79 (SEQ ID NO: 118);

Junction 19 (SEQ ID NO:37-38) can be used to join DHR54 (SEQ ID NO: 104)-DHR79 (SEQ ID NO: 118);

Junction 20 (SEQ ID NO:39-40) can be used to join DHR79 (SEQ ID NO: 118)-DHR14 (SEQ ID NO: 86);

Junction 21 (SEQ ID NO:41-42) can be used to join DHR79 (SEQ ID NO: 118)-DHR14 (SEQ ID NO: 86);

Junction 22 (SEQ ID NO:43-44) can be used to join DHR79 (SEQ ID NO: 118)-DHR54 (SEQ ID NO: 104);

Junction 23 (SEQ ID NO:45-46) can be used to join DHR14 (SEQ ID NO:86)-DHR18 (SEQ ID NO: 88);

Junction 24 (SEQ ID NO:47-48) can be used to join DHR14 (SEQ ID NO:86)-DHR18 (SEQ ID NO: 88);

Junction 25 (SEQ ID NO:49-50) can be used to join DHR14 (SEQ ID NO:86)-DHR54 (SEQ ID NO: 104);

Junction 26 (SEQ ID NO:51-52) can be used to join DHR14 (SEQ ID NO:86)-DHR71 (SEQ ID NO: 113);

Junction 27 (SEQ ID NO:53-54) can be used to join DHR14 (SEQ ID NO:86)-DHR79 (SEQ ID NO: 118);

Junction 28 (SEQ ID NO:55-56) can be used to join DHR14 (SEQ ID NO:86)-DHR79 (SEQ ID NO: 118);

Junction 29 (SEQ ID NO:57-58) can be used to join DHR14 (SEQ ID NO:86)-DHR8 (SEQ ID NO: 83);

Junction 30 (SEQ ID NO:59-60) can be used to join DHR14 (SEQ ID NO:86)-DHR8 (SEQ ID NO: 83);

Junction 31 (SEQ ID NO:61-62) can be used to join DHR14 (SEQ ID NO:86)-DHR8 (SEQ ID NO: 83);

Junction 32 (SEQ ID NO:63-64) can be used to join DHR49 (SEQ ID NO: 101)-DHR79 (SEQ ID NO: 118);

Junction 33 (SEQ ID NO:65-66) can be used to join DHR4 (SEQ ID NO: 80)-DHR64 (SEQ ID NO: 110); and

Junction 34 (SEQ ID NO:67-68) can be used to join DHR53 (SEQ ID NO: 103)-DHR4 (SEQ ID NO: 80).

Thus, if X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to DHR4 (SEQ ID NO:80), then X4 may be (for example) a junction polypeptide at least 50%, 55%, 60%, 65%, 70%, 75%, 80%, 85%, 90%, 91%, 92%, 93%, 94%, 95%, 96%, 97%, 98%, 99%, or 100% identical to junction polypeptide 33 (SEQ ID NO:65 or 66).

Similarly, if X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to DHR49 (SEQ ID NO:101), then X4 may be (for example) a junction polypeptide at least 50%, 55%, 60%, 65%, 70%, 75%, 80%, 85%, 90%, 91%, 92%, 93%, 94%, 95%, 96%, 97%, 98%, 99%, or 100% identical to junction polypeptide 32 (SEQ ID NO:63 or 64).

In light of these exemplary embodiments, those of skill in the art will understand the numerous other embodiments contemplated by the recitation that X4 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to a junction polypeptide that can be used to form a junction with X3 as shown in Table 1.

In some embodiments, X4 is at least 50%, 55%, 60%, 65%, 70%, 75%, 80%, 85%, 90%, 91%, 92%, 93%, 94%, 95%, 96%, 97%, 98%, 99%, or 100% identical to the junction polypeptide of X2. Thus, in some embodiments the X2 junction polypeptide may be identical to the X4 junction polypeptide; in other embodiments it may be related but containing modifications relative to the X4 junction polypeptide.

In a further embodiment, the fusion protein comprises the general formula X1-X2-X3-X4-X5, wherein X5 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to a DHR polypeptide that can be used with the X4 junction as shown in Table 1. As noted above, the fusion proteins can be linked together in various combinations for form polymers. By way of non-limiting example, if X4 is a junction polypeptide at least 50%, 55%, 60%, 65%, 70%, 75%, 80%, 85%, 90%, 91%, 92%, 93%, 94%, 95%, 96%, 97%, 98%, 99%, or 100% identical to junction polypeptide 33 (SEQ ID NO:65 or 66), then X5 may be (for example), a DHR polypeptide at least 50%, 55%, 60%, 65%, 70%, 75%, 80%, 85%, 90%, 91%, 92%, 93%, 94%, 95%, 96%, 97%, 98%, 99%, or 100% identical to DHR64 (SEQ ID NO: 110). In light of this exemplary embodiments, those of skill in the art will understand the numerous other embodiments contemplated by the recitation that X5 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to a DHR polypeptide that can be used with the X4 junction as shown in Table 1.

Furthermore, those of skill in the art will understand that the various junction polypeptides and DHR polypeptides may be continually combined to produce a polymer of any number of X₍ₙ₎ domains as deemed appropriate for an intended use.

In another embodiment, the disclosure provides polypeptides comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NOS:121-142, wherein residues in parentheses are optional and may be present or absent. Exemplary such polypeptides are shown in Table 2, representing fusion proteins capable of forming polymers as described in detail herein.

TABLE 2

| name | Sequence |
|---|---|
| Sculpt 35 | MSAEKLMLMAKLIIIVAENAKRKGDDTLIAIMAAKLAFEIVRIAAEEAGIDSSEVLELAIRLIKEVVENAQREGYD |
| HR10C5_2- | ISIAALAAAMAFALVAIAAKRAGITSPEVLKLAIILIKLVVLAAQLSGYDIEEAAKKAAETFLRVAEEAREKGIDP |
| (DHR10-DHR14) | REVIARSIADAAEEAATLAVRKGDEESLKSIVRLAATAAKTAKNPEVITKIVNLLLEIAERATDNELVNEIVKQLA |
| -DHR14₃ | EVAKEATDKDLVIHIVRILAELAKHSTDSELVNEIVKQLAEVAKRATDKELVIEIVRILAELAKESTDSRLVEEIV |
| | RQLKEVAERATDKELVEEIEKILEELKKESTDGWLEHHHHHH (SEQ ID NO: 121) |
| | (M)SAEKLMLMAKLIIIVAENAKRKGDDTLIAIMAAKLAFEIVRIAAEEAGIDSSEVLELAIRLIKEVVENAQREG |
| | YDISIAALAAAMAFALVAIAAKRAGITSPEVLKLAIILIKLVVLAAQLSGYDIEEAAKKAAETFLRVAEEAREKGI |
| | DPREVIARSIADAAEEAATLAVRKGDEESLKSIVRLAATAAKTAKNPEVITKIVNLLLEIAERATDNELVNEIVKQ |
| | LAEVAKEATDKDLVIHIVRILAELAKHSTDSELVNEIVKQLAEVAKRATDKELVIEIVRILAELAKESTDSRLVEE |
| | IVRQLKEVAERATDKELVEEIEKILEELKKESTD(GWLEHHHHHH) (SEQ ID NO: 122) |
| Sculpt 36 | MHHHHHHHGGSGENLYFQGGSGWGNEEEEKLKELLERAKELAKSPDPEDLKEAVRLAEEVVRERPGSEAAKKALEI |
| (DHR53-DHR4) | IQEAAELLKESPDPEAIIAAARALLKIAATTGDEEAAKEAIEAAEKAARLAEERGDDELVCEALALLIAARVLLLK |
| -DHR4₂ | QQGTSDEEVAETVARTISKLVKRLKKKGASEEVICECVARIVAEIVKALKRSGTSEEEIAEIVARVISEVIRTLEE |
| -HR04C4_1 | SGSSYEVICECVARIVAEIVEALKRSGTSAVEIAKIVARVISEVIRTLKESGSSYEVICECVARIVAEIVEALKRS |
| | GTSAAIIALIVALVISEVIRTLKESGSSFEVILECVIRIVLEIIEALKRSGTSEQDVMLIVMAVLLVVLATLQLSG |
| | S (SEQ ID NO: 123) |
| | (MHHHHHHHGGSGENLYFQGGSGWG)NEEEEKLKELLERAKELAKSPDPEDLKEAVRLAEEVVRERPGSEAAKKAL |
| | EIIQEAAELLKESPDPEAIIAAARALLKIAATTGDEEAAKEAIEAAEKAARLAEERGDDELVCEALALLIAARVLL |
| | LKQQGTSDEEVAETVARTISKLVKRLKKKGASEEVICECVARIVAEIVKALKRSGTSEEEIAEIVARVISEVIRTL |
| | EESGSSYEVICECVARIVAEIVEALKRSGTSAVEIAKIVARVISEVIRTLKESGSSYEVICECVARIVAEIVEALK |
| | RSGTSAAIIALIVALVISEVIRTLKESGSSFEVILECVIRIVLEIIEALKRSGTSEQDVMLIVMAVLLVVLATLQL |
| | SGS(SEQ ID NO: 124) |
| Sculpt 37 | MSAEKLMLMAKLIIIVAENAKRKGDDTLIAIMAAKLAFEIVRIAAEEAGIDSSEVLELAIRLIKEVVENAQREGYD |
| HRI0C5_2 | ISIAALAAAMAFALVAIAAKRAGITSSEVLELAIRLIKKVVENAQREGYDIEEAARAAAEAFERVAEAAKRAGITS |
| -(DHR10-DHR9) | SKAIKIAIELIEVVVRAASRNGHDISKAARKAAETIKTAADLAKKGNPDELAKHIAKTVEELKRNGVSEDEIARTV |
| -DHR9₃ | AAIIAFVIQALKSSGSSEDVIATIVARIVAEIVRALKRSGTSEDEIAEIVAKVISEVIRTLKESGSSHEVIAKIVA |
| | RIVAEIVEALKDSGTSEEEIAKIVAHVISEVIRTLKESGSSEEVIHHIVKRIVHEIVKALKESGTSEDEIREIVKH |
| | VEHEVERTLHESGSSGWLEHHHHHH (SEQ ID NO: 125) |
| | (M)SAEKLMLMAKLIIIVAENAKRKGDDTLIAIMAAKLAFEIVRIAAEEAGIDSSEVLELAIRLIKEVVENAQREG |
| | YDISIAALAAAMAFALVAIAAKRAGITSSEVLELAIRLIKKVVENAQREGYDIEEAARAAAEAFERVAEAAKRAGI |
| | TSSKAIKIAIELIEVVVRAASRNGHDISKAARKAAETIKTAADLAKKGNPDELAKHIAKTVEELKRNGVSEDEIAR |
| | TVAAIIAFVIQALKSSGSSEDVIATIVARIVAEIVRALKRSGTSEDEIAEIVAKVISEVIRTLKESGSSHEVIAKI |
| | VARIVAEIVEALKDSGTSEEEIAKIVAHVISEVIRTLKESGSSEEVIHHIVKRIVHEIVKALKESGTSEDEIREIV |
| | KHVEHEVERTLHESGSS(GWLEHHHHHH) (SEQ ID NO: 126) |
| Sculpt 38 | MHHHHHHHGGSGENLYFQGGSGWGSEEVNERVKQLAEKAKEATDKEEVIEIVKELAELAKQSTDPNLVAEVVRALT |
| (DHR14-DHR18) | EVAKTSTDTELIREIIKVLLELASKLRDPQAVLEALQAVAELARELAEKTGDPIAKLCAIAVSLAAEAVKKAAELL |
| -tj18C2_V03 | KRHPDSQAAQDALKLAKQAAEAVLLACLLALEHPNAIIAILCIVAAIAAAIAASMAAALAQRHPDSQAARDAIKLA |
| | SQAAEAVKLACELAQEHPNAKIAVLCILAAALAAIAAALAALLAQLHPDSQAARDAIKLASQAAEAVKLACELAQE |
| | HPNADIAEKCILLAILAALLAILAALLAMLHPDSQLARDLIDLASELAEEVKERCER (SEQ ID |
| | NO: 127) |
| | (MHHHHHHHGGSGENLYFQGGSGWG)SEEVNERVKQLAEKAKEATDKEEVIEIVKELAELAKQSTDPNLVAEVVRA |
| | LTEVAKTSTDTELIREIIKVLLELASKLRDPQAVLEALQAVAELARELAEKTGDPIAKLCAIAVSLAAEAVKKAAE |
| | LLKRHPDSQAAQDALKLAKQAAEAVLLACLLALEHPNAIIAILCIVAAIAAAIAASMAAALAQRHPDSQAARDAIK |
| | LASQAAEAVKLACELAQEHPNAKIAVLCILAAALAAIAAALAALLAQLHPDSQAARDAIKLASQAAEAVKLACELA |
| | QEHPNADIAEKCILLAILAALLAILAALLAMLHPDSQLARDLIDLASELAEEVKERCER (SEQ ID |
| | NO: 128) |
| Sculpt 39 | MHHHHHHHGGSGENLYFQGGSGWGNEEEEHLKELLKRAEELAKSPDPDDLREAVRLAEEVVRTRPGSELAKKALEI |
| DHR53₂ | ILRAAEELAKLPDPEALHEAVRAAEHVVRSQPGSEAAKEALRIIQEAAELLKESPDPTAIIRAARALLKIARTTGD |
| -(DHR53-DHR4) | EEAAKEAIEAAKKAADLARERGDDELVCEALALLVAAQVELLKQQGTSAVEIAKIVARVISEVIRTLKEKGSSYEV |
| -HR04C4_l | ICECVARIVAEIVEALKRSGTSAAIIALIVALVISEVIRTLKESGSSFEVILECVIRIVLEIIEALKRSGTSEQDV |
| | MLIVMAVLLVVLATLQLSGS (SEQ ID NO: 129) |
| | (MHHHHHHHGGSGENLYFQGGSGWG)NEEEEHLKELLKRAEELAKSPDPDDLREAVRLAEEVVRTRPGSELAKKAL |
| | EIILRAAEELAKLPDPEALHEAVRAAEHVVRSQPGSEAAKEALRIIQEAAELLKESPDPTAIIRAARALLKIARTT |
| | GDEEAAKEAIEAAKKAADLARERGDDELVCEALALLVAAQVELLKQQGTSAVEIAKIVARVISEVIRTLKEKGSSY |
| | EVICECVARIVAEIVEALKRSGTSAAIIALIVALVISEVIRTLKESGSSFEVILECVIRIVLEIIEALKRSGTSEQ |
| | DVMLIVMAVLLVVLATLQLSGS (SEQ ID NO: 130) |
| Sculpt 40 | MHHHHHHHGGSGENLYFQGGSGWGSELGKRLIEAAENGNKDRVKDLIENGADVNASDSDGRTPLHHAAENGHKEVV |
| (ankl-DHR18 ) | KLLISKGADVNAKDSDGRTPLHHAAENGHKEVVKLLISKGADVNAKDSDGRTPLHHAAENGHKEVVKLLISKGADV |
| -DHR18₂ | NAKADRGMTPLHFAAWRGHKEVVKLLISKGADLNTSAKDGATPVLLALRRGDEEVVRLLKEEAKKRGDEFLARCAE |
| -tj18C2_V03 | AAELAIEALKLAEELLRRYPNDEAARLAHHLAKLALEAVELACILASEHPNADIAKLCIKAASEAAEAASKAAELA |
| | QRHPDSQAARDAIKLASQAAEAVKLACELAQEHPNADIAKLCIIAASLAAEAASKAAELAQRHPDSQAARDAIKLA |
| | SQAAEAVKLACELAQEHPNAIIAILCIVAAIAAAIAASMAAALAQRHPDSQAARDAIKLASQAAEAVKLACELAQE |
| | HPNAKIAVLCILAAALAAIAAALAALLAQLHPDSQAARDAIKLASQAAEAVKLACELAQEHPNADIAEKCILLAIL |
| | AALLAILAALLAMLHPDSQLARDLIDLASELAEEVKERCER (SEQ ID NO: 131) |
| | (MHHHHHHHGGSGENLYFQGGSGWG)SELGKRLIEAAENGNKDRVKDLIENGADVNASDSDGRTPLHHAAENGHKE |
| | VVKLLISKGADVNAKDSDGRTPLHHAAENGHKEVVKLLISKGADVNAKDSDGRTPLHHAAENGHKEVVKLLISKGA |
| | DVNAKADRGMTPLHFAAWRGHKEVVKLLISKGADLNTSAKDGATPVLLALRRGDEEVVRLLKEEAKKRGDEFLARC |
| | AEAAELAIEALKLAEELLRRYPNDEAARLAHHLAKLALEAVELACILASEHPNADIAKLCIKAASEAAEAASKAAE |
| | LAQRHPDSQAARDAIKLASQAAEAVKLACELAQEHPNADIAKLCIIAASLAAEAASKAAELAQRHPDSQAARDAIK |
| | LASQAAEAVKLACELAQEHPNAIIAILCIVAAIAAAIAASMAAALAQRHPDSQAARDAIKLASQAAEAVKLACELA |
| | QEHPNAKIAVLCILAAALAAIAAALAALLAQLHPDSQAARDAIKLASQAAEAVKLACELAQEHPNADIAEKCILLA |
| | ILAALLAILAALLAMLHPDSQLARDLIDLASELAEEVKERCER (SEQ ID NO: 132) |
| Sculpt 41 | MIEEVVAEMIDILAESSKKSIEELARAADNKTTEKAVAEAIEEIARLATAAIQLIEALAKNLASEEFMARAISAIA |
| (139_tj41C3_ | ELAKKAIEAIYRLADNHTTDTFMARAIAAIANLAVTAILAIAALASNHTTEEFMARAISAIAELAKKAIEAIYRLA |
| pmlv2_DHR27) | DNHTTDKFMAAAIEAIALLATLAILAIALLASNHTTEEFMAKAISAIAELAKKAIEAIYRLADNHTNEELIRHAIE |
| tj4lC3_pmlv2 | IIREIAEIAARAIIEIAKRLKSEEYALHALRAVLEIIEHALERIARKADKEEKKALELLIEVAREIYRLAEEAAKR |
| -(TJ41-DHR27) | AKDEEEAAKIAVIAAEAILELLRAQRKVTDNEVIEKLLEVVKEIIRLAEEAMKKMTDEEEAAKIAKEALEAIKMLA |
| | RAVEEVTDNEVIEKLLEVVKEIIRLAEEAMKKMTDEEEAAKIAKEALEAIKMLARAVEEVTDKERIEQLLREVKEE |
| | IRRAEEESRKETDDEEAAKRAREALRRIRERAREVEEDKSGWLEHHHHHH (SEQ ID NO: 133) |
| | (M)IEEVVAEMIDILAESSKKSIEELARAADNKTTEKAVAEAIEEIARLATAAIQLIEALAKNLASEEFMARAISA |
| | IAELAKKAIEAIYRLADNHTTDTFMARAIAAIANLAVTAILAIAALASNHTTEEFMARAISAIAELAKKAIEAIYR |
| | LADNHTTDKFMAAAIEAIALLATLAILAIALLASNHTTEEFMAKAISAIAELAKKAIEAIYRLADNHTNEELIRHA |
| | IEIIREIAEIAARAIIEIAKRLKSEEYALHALRAVLEIIEHALERIARKADKEEKKALELLIEVAREIYRLAEEAA |
| | KRAKDEEEAAKIAVIAAEAILELLRAQRKVTDNEVIEKLLEVVKEIIRLAEEAMKKMTDEEEAAKIAKEALEAIKM |
| | LARAVEEVTDNEVIEKLLEVVKEIIRLAEEAMKKMTDEEEAAKIAKEALEAIKMLARAVEEVTDKERIEQLLREVK |
| | EEIRRAEEESRKETDDEEAAKRAREALRRIRERAREVEEDKS(GWLEHHHHHH) (SEQ ID NO: 134) |
| Sculpt 42 | MIEEVVAEMIDILAESSKKSIEELARAADNKTTEKAVAEAIEEIARLATAAIQLIEALAKNLASEEFMARAISAIA |
| tj4lC3_pmlv2 | ELAKKAIEAIYRLADNHTTDTFMARAIAAIANLAVTAILAIAALASNHTTEEFMARAISAIAELAKKAIEAIYRLA |
| -(TJ41-DHR1) | DNHTTDKFMAAAIEAIALLATLAILAIALLASNHTTEEFMAKAISAIAELAKKAIEAIYRLADNHTNEEAIHEAAE |
| | AILRIAEEAIRAIEELVRRSKSEEIEERAKKLIEEIARKAIEAALRLGSEEIAARVAYILIEIIIKRHPGDKEEAA |
| | EIARKIIEQIIRTLPGGCDCVAKAASSIIRAVIEKNPNYSEVVADVAAAIVKAIIEGNPNGCDCVAKAASSIIRAV |
| | IEKNPNYSEVVADVAAAIVKAIIEGNPNGRDCVRKAASSIIRAVQEKNPNYSEVVEDVKRAIEKAIKEGNPNGGWL |
| | EHHHHHH (SEQ ID NO: 135) |
| | (M)IEEVVAEMIDILAESSKKSIEELARAADNKTTEKAVAEAIEEIARLATAAIQLIEALAKNLASEEFMARAISA |
| | IAELAKKAIEAIYRLADNHTTDTFMARAIAAIANLAVTAILAIAALASNHTTEEFMARAISAIAELAKKAIEAIYR |
| | LADNHTTDKFMAAAIEAIALLATLAILAIALLASNHTTEEFMAKAISAIAELAKKAIEAIYRLADNHTNEEAIHEA |
| | AEAILRIAEEAIRAIEELVRRSKSEEIEERAKKLIEEIARKAIEAALRLGSEEIAARVAYILIEIIIKRHPGDKEE |
| | AAEIARKIIEQIIRTLPGGCDCVAKAASSIIRAVIEKNPNYSEVVADVAAAIVKAIIEGNPNGCDCVAKAASSIIR |
| | AVIEKNPNYSEVVADVAAAIVKAIIEGNPNGRDCVRKAASSIIRAVQEKNPNYSEVVEDVKRAIEKAIKEGNPNG( |
| | GWLEHHHHHH) (SEQ ID NO: 136) |
| Sculpt 43 | MIEEVVAEMIDILAESSKKSIEELARAADNKTTEKAVAEAIEEIARLATAAIQLIEALAKNLASEEFMARAISAIA |
| HR10C5_2 | ELAKKAIEAIYRLADNHTTDTFMARAIAAIANLAVTAILAIAALASNHTTEEFMARAISAIAELAKKAIEAIYRLA |
| -(DHR10-DHR39) | DNHTTDKFMAAAIEAIALLATLAILAIALLASNHTTEEFMAKAISAIAELAKKAIEAIYRLADNHTNEEAIHEAAE |
| -DHRsc39₄ | AILRIAEEAIRAIEELVRRSKSEEIEERAKKLIEEIARKAIEAALRLGSEEIAARVAYILIEIIIKRHPGDKEEAA |
| | EIARKIIEQIIRTLPGGCDCVAKAASSIIRAVIEKNPNYSEVVADVAAAIVKAIIEGNPNGCDCVAKAASSIIRAV |
| | IEKNPNYSEVVADVAAAIVKAIIEGNPNGRDCVRKAASSIIRAVQEKNPNYSEVVEDVKRAIEKAIKEGNPNGGWL |
| | EHHHHHH (SEQ ID NO: 137) |
| | (M)IEEVVAEMIDILAESSKKSIEELARAADNKTTEKAVAEAIEEIARLATAAIQLIEALAKNLASEEFMARAISA |
| | IAELAKKAIEAIYRLADNHTTDTFMARAIAAIANLAVTAILAIAALASNHTTEEFMARAISAIAELAKKAIEAIYR |
| | LADNHTTDKFMAAAIEAIALLATLAILAIALLASNHTTEEFMAKAISAIAELAKKAIEAIYRLADNHTNEEAIHEA |
| | AEAILRIAEEAIRAIEELVRRSKSEEIEERAKKLIEEIARKAIEAALRLGSEEIAARVAYILIEIIIKRHPGDKEE |
| | AAEIARKIIEQIIRTLPGGCDCVAKAASSIIRAVIEKNPNYSEVVADVAAAIVKAIIEGNPNGCDCVAKAASSIIR |
| | AVIEKNPNYSEVVADVAAAIVKAIIEGNPNGRDCVRKAASSIIRAVQEKNPNYSEVVEDVKRAIEKAIKEGNPNG( |
| | GWLEHHHHHH) (SEQ ID NO: 138) |
| Sculpt 44 | HHHHHHHGGSGENLYFQGGSGWGSEEVNKKVEDLAREAQKATDKETVIRIVETLAELAKKSTDKDLVNEIVRQLAE |
| DHR14₉ | VAKQATDKELVIRIVEILAELAKTSTDSELVNEIVKQLAEVAKRATDPELVIRIVEILAELAKTSTDSELVNEIVK |
| -(DHR14-DHR76) | QLAEVAKRATDPDLVIYIVTILAELAKTSTDKDLVNEIVKQLAEVAKRATDKDLVIYIVTILAELAKTSTDSKLVE |
| -DHR76₉ | EIVKQLAEVAKRATDKELVIYIVTILAELAKTSTDSELVNEIVKQLAEVAKRATDKELVIYIVHILARLAQTSTDS |
| | ELVNEIVKQLAEVAKRATDKELVIYIVEILARLADTSTDQELVRRIVQQLAQVAKRATDNELVIYIVEILAELAKR |
| | STDPKVVAEILQALAEVAQQSTDPELARKIIEVIAELAKDQGDSALLQAAEAAKKAANKGNERLLLAVLQALLVAV |
| | EVLIVAEEARENGNKELADAATRLIKAVARAITEAVDQGNPELVKWVAEAAKVAADVIRVAIQANREGNSQLFKAA |
| | LRLVEAVIEAIKEAVDQGNPELVHWVARAAKVAADVIRVAIQAKKEGNEELFQAALRLVQAVIEAIKEAVKQGNPE |
| | LVEWVARAAKVAADVIRVAIQAKREGNRELFEAALRLVQAVIEAIKEAVKQGNPELVEWVARAAKVAAEVIKVAIQ |
| | AKREGNEELFQAALRLVQAVIEAIKEAVKQGNPELVEWVARAATVAAEVIKVAIQAKKEGNPDLFRAALRLVDAVI |
| | EAIKRAVKQGNPELVEWVARAAHVAARVIEVAIQAKREGNPELFKAALRLVDAVIEAIKRAVRQGNPELVEWVARA |
| | AKVAAEVIKVAIQAKKEGNRELFEAALRLVDAVIEAIKRAVRQGNPELVEWVARAAHVAARVIEVAIQAKKEGNPD |
| | LFRAALRLVQAVIEAIKEAVRQGNPELVERVARLATHAAELIKEAIKAKREGNDDKRRRALETVQKVIEDIKELVR |
| | QGN (SEQ ID NO: 139) |
| | (HHHHHHHGGSGENLYFQGGSGWG)SEEVNKKVEDLAREAQKATDKETVIRIVETLAELAKKSTDKDLVNEIVRQL |
| | AEVAKQATDKELVIRIVEILAELAKTSTDSELVNEIVKQLAEVAKRATDPELVIRIVEILAELAKTSTDSELVNEI |
| | VKQLAEVAKRATDPDLVIYIVTILAELAKTSTDKDLVNEIVKQLAEVAKRATDKDLVIYIVTILAELAKTSTDSKL |
| | VEEIVKQLAEVAKRATDKELVIYIVTILAELAKTSTDSELVNEIVKQLAEVAKRATDKELVIYIVHILARLAQTST |
| | DSELVNEIVKQLAEVAKRATDKELVIYIVEILARLADTSTDQELVRRIVQQLAQVAKRATDNELVIYIVEILAELA |
| | KRSTDPKVVAEILQALAEVAQQSTDPELARKIIEVIAELAKDQGDSALLQAAEAAKKAANKGNERLLLAVLQALLV |
| | AVEVLIVAEEARENGNKELADAATRLIKAVARAITEAVDQGNPELVKWVAEAAKVAADVIRVAIQANREGNSQLFK |
| | AALRLVEAVIEAIKEAVDQGNPELVHWVARAAKVAADVIRVAIQAKKEGNEELFQAALRLVQAVIEAIKEAVKQGN |
| | PELVEWVARAAKVAADVIRVAIQAKREGNRELFEAALRLVQAVIEAIKEAVKQGNPELVEWVARAAKVAAEVIKVA |
| | IQAKREGNEELFQAALRLVQAVIEAIKEAVKQGNPELVEWVARAATVAAEVIKVAIQAKKEGNPDLFRAALRLVDA |
| | VIEAIKRAVKQGNPELVEWVARAAHVAARVIEVAIQAKREGNPELFKAALRLVDAVIEAIKRAVRQGNPELVEWVA |
| | RAAKVAAEVIKVAIQAKKEGNRELFEAALRLVDAVIEAIKRAVRQGNPELVEWVARAAHVAARVIEVAIQAKKEGN |
| | PDLFRAALRLVQAVIEAIKEAVRQGNPELVERVARLATHAAELIKEAIKAKREGNDDKRRRALETVQKVIEDIKEL |
| | VRQGN (SEQ ID NO: 140) |
| Sculpt 45 | HHHHHHHGGSGENLYFQGGSGWGDSEEVNDKVRRLAKKAKDATDKETVIRIVHTLARLAEKSTDKDLVNEIVKQLA |
| DHR14₇ | EVAKRATDKELVIRIVEILARLAERSTDSELVNEIVKQLAEVAKRATDQELVIRIVEILAELAKRSTDKDLVNEIV |
| -(DHR14-DHR79) | KQLAEVAKRATDQDLVIRIVEILAELAKTSTDKDLVNEIVKQLAEVAKRATDPDLVIRIVEILAELAKTSTDSKLV |
| -(DHR79-DHR54) | NDIVKQLAEVAKRATDKDLVIRIVHILHRLAQTSTDDELVNEIVRQLAEVARRATDRELVIHIVTILAKLAEESTD |
| -DHR54₇ | EKAIQEIAERLATVAKESQDEELILTIILVLLRLLSTSTDPEALEQIARAVLELARQNGDEKLAELAEEALRAVQT |
| | AKEAKEKGDEDLAQAALLIALAAAAAAAALIAARQTGDPRVRRLAEELKRLAQEAAERVKRDPSSEETLRALTIII |
| | IAIEVAVIALEVARKQGNPNVKRRASELVEQAVRAAQEVNDDPTDEAVYNAVHTLARAALQAVKDGPDTQEVVKKA |
| | LEVVAKLAIIAARQGSTDAVRDALQVALEIARTAGNQEAVKLALEVVAQVAIEAAKTGNTDAVREALRVALQIART |
| | SGTEEAVKLALEVVARVAIEAARRGNTDAVRDALEVALQIARTSGTEEAVKLALEVVARVAIEAARRGNTEAVREA |
| | LEVALKIAKTSGTQEAVKLALEVVARVAIEAARRGNTEAVRDALRVALKIAKTSGTEEAVKLALEVVARVAIEAAR |
| | RGNTDAVRDALQVALEIAKTSGTEEAVKLALEVVARVAIEAARRGNTDAVREALEVALQIARTSGTDEAVKLALEV |
| | VKRVSDEARRRGNEEAVKEAEEVRERIERTQGT (SEQ ID NO: 141) |
| | (HHHHHHHGGSGENLYFQGGSGWG)DSEEVNDKVRRLAKKAKDATDKETVIRIVHTLARLAEKSTDKDLVNEIVKQ |
| | LAEVAKRATDKELVIRIVEILARLAERSTDSELVNEIVKQLAEVAKRATDQELVIRIVEILAELAKRSTDKDLVNE |
| | IVKQLAEVAKRATDQDLVIRIVEILAELAKTSTDKDLVNEIVKQLAEVAKRATDPDLVIRIVEILAELAKTSTDSK |
| | LVNDIVKQLAEVAKRATDKDLVIRIVHILHRLAQTSTDDELVNEIVRQLAEVARRATDRELVIHIVTILAKLAEES |
| | TDEKAIQEIAERLATVAKESQDEELILTIILVLLRLLSTSTDPEALEQIARAVLELARQNGDEKLAELAEEALRAV |
| | QTAKEAKEKGDEDLAQAALLIALAAAAAAAALIAARQTGDPRVRRLAEELKRLAQEAAERVKRDPSSEETLRALTI |
| | IIIAIEVAVIALEVARKQGNPNVKRRASELVEQAVRAAQEVNDDPTDEAVYNAVHTLARAALQAVKDGPDTQEVVK |
| | KALEVVAKLAIIAARQGSTDAVRDALQVALEIARTAGNQEAVKLALEVVAQVAIEAAKTGNTDAVREALRVALQIA |
| | RTSGTEEAVKLALEVVARVAIEAARRGNTDAVRDALEVALQIARTSGTEEAVKLALEVVARVAIEAARRGNTEAVR |
| | EALEVALKIAKTSGTQEAVKLALEVVARVAIEAARRGNTEAVRDALRVALKIAKTSGTEEAVKLALEVVARVAIEA |
| | ARRGNTDAVRDALQVALEIAKTSGTEEAVKLALEVVARVAIEAARRGNTDAVREALEVALQIARTSGTDEAVKLAL |
| | EVVKRVSDEARRRGNEEAVKEAEEVRERIERTQGT (SEQ ID NO: 142) |

In an embodiment all aspects and embodiments of the polypeptides and fusion proteins disclosed herein, a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that the desired activity is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In one embodiment, mutations in hydrophobic residues relative to the reference sequence are conservative amino acid substitutions. In another embodiment, mutations in residues relative to the reference sequence are conservative amino acid substitutions.

In one embodiment 1, 2, 3, 4, 5, 6, 7, 8, or more of the optional amino acid residues are absent. In another embodiment, 1, 2, 3, 4, 5, 6, 7, 8, or more of the optional amino acid residues are present. In a further embodiment, all of the optional amino acid residues are absent. In one embodiment, all of the optional amino acid residues are present.

In another embodiment, the disclosure provides polymers comprising the polypeptides or fusion proteins of the disclosure. As noted above, the polypeptides and fusion proteins may be joined in numerous configurations to generate polymers of interest. In various embodiments, the polymer comprises 3, 4, 5, 6, 7, 8, 9, 10, 15, 20, 25, 50, 75, 100, or more copies or repeats of the fusion protein or polypeptides of the disclosure.

In another embodiment, the polymers further comprise one or more functional molecules bound to the polymer. The functional molecules can be bound to the polypeptides or fusion proteins via genetic fusion prior to forming the polymers, may be covalently attached to formed polymers, or may be bound to the polymers via any other suitable means. Any suitable functional molecule may be bound to the polymer as deemed appropriate for an intended use, including but not limited to receptor binding domains, detectable molecules, antibodies, mini-protein binders, ankyrins, and/or protein A.

In another embodiment, the disclosure provides a composition, comprising 5, 10, 25, 50, 75, 100, 250, 500, 1000, or more different polypeptides, fusion proteins, and/or polymers of any embodiment or combination of embodiments disclosed herein.

As used throughout the present application, the term “polypeptide” is used in its broadest sense to refer to a sequence of subunit amino acids. The polypeptides of the invention may comprise L-amino acids, D-amino acids (which are resistant to L-amino acid-specific proteases in vivo), or a combination of D- and L-amino acids. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

As will be understood by those of skill in the art, the polypeptides of the invention may include additional residues at the N-terminus, C-terminus, or both that are not present in the polypeptides disclosed herein; these additional residues are not included in determining the percent identity of the polypeptides of the invention relative to the reference polypeptide.

In another aspect the disclosure provides nucleic acids encoding the polypeptide or fusion protein of any embodiment or combination of embodiments of the disclosure. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids, expression vectors (i.e.: episomal or chromosomally integrated), polypeptides, fusion protein, or compositions disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another aspect, the disclosure provides methods for designing rigid helical junctions for modular repeat proteins, or methods for designing a non-natural modular repeat protein comprising rigid helical junctions, using any method or combination of methods as disclosed in the examples that follow. In one embodiment the protein comprises at least 1, 2, or 3 polypeptides selected from:

a) a de novo helical repeat building block polypeptide;

b) homo-oligomer polypeptide; and

c) ankyrin polypeptide.

In another embodiment, junctions between the polypeptide and neighboring amino acid sequence comprises an overlap of six (6) amino acid residues. In a further embodiment, the protein comprises two or more helices in contact throughout the rigid helical junction, and there are no buried unsatisfied residues.

The disclosure further provides non-natural modular repeat proteins comprising rigid helical junctions, for example those made using the design methods described herein. In one embodiment, the protein comprises at least one de novo helical repeat building block polypeptide. In another embodiment, the protein comprises at least one homo-oligomer polypeptide. In one embodiment, the protein comprises at least one ankyrin polypeptide. In another embodiment, the protein comprises at least two of:

a) a de novo helical repeat building block polypeptide;

b) homo-oligomer polypeptide; and

c) ankyrin polypeptide.

### Examples

### Abstract:

The ability to precisely design large proteins with diverse shapes would enable applications ranging from the design of protein binders that wrap around their target to the positioning of multiple functional sites in specified orientations. We describe a protein backbone design method for generating a wide range of rigid fusions between helix containing proteins, and use it to design 75,000 structurally unique junctions between monomeric and homo-oligomeric de novo designed and ankyrin repeat proteins. Of the junction designs that were experimentally characterized, 82% have circular dichroism and solution x-ray scattering profiles consistent with the design models and are stable at 95° C. Crystal structures of 4 designed junctions were in close agreement with the design models with RMSDs ranging from 0.9 to 1.6 Å. Electron microscopic images of extended tetrameric structures and ˜10 nm diameter “L” and “V” shapes generated using the junctions are close to the design models, demonstrating the control the rigid junctions provide for protein shape sculpting over multiple nanometer length scales.

The ability to robustly control macromolecular shape on the nanometer length scale is important for a wide range of biomedical and materials applications. We describe a large library of protein building blocks and junctions between them that enable the design of proteins with a wide range of shapes through modular combination of blocks rather than traditional and more complex design at the level of amino acid residues.

### Introduction

A modular combination of structured elements is difficult with proteins because they can adopt a wide variety of folds that are not universally complementary. The rigid body orientation of multiple protein domains with flexible linkers is not fixed, making it difficult to programmatically assemble larger structures using this approach. The design of complex structures would be considerably facilitated by general methods for rigidly fusing together pre-existing modules.

Here we focus on the creation of a wide range of protein shapes using a diverse set of de novo designed protein building blocks with structural features that enable rigid fusion. Repeat proteins are excellent building blocks for protein-based nano-scale materials as they can readily be shortened or lengthened by changing the number of repeats; hence each repeat protein generates a family of structures RPₙ, where n is the number of repeats. A rigid fusion of two different repeat proteins would provide access to the larger family of structures RP1ₘRP2ₙ, and fusion of three to the still larger family RP1ₘRP2ₙRP3ₗ. The set of de novo designed helical repeat proteins (DHRs) is a particularly attractive starting point: DHRs are extremely stable with individual repeat units that, unlike the repeat proteins in nature, have favorable folding free energies⁷ and are identical in each copy in the overall protein. 44 DHRs have been structurally validated: 15 by crystallography and the remainder by solution x-ray scattering (SAXS). The DHRs are quite versatile: they have been built into homo-oligomers, filaments, lattices on inorganic crystals, and used as scaffolds for ligand induced heterodimerization.

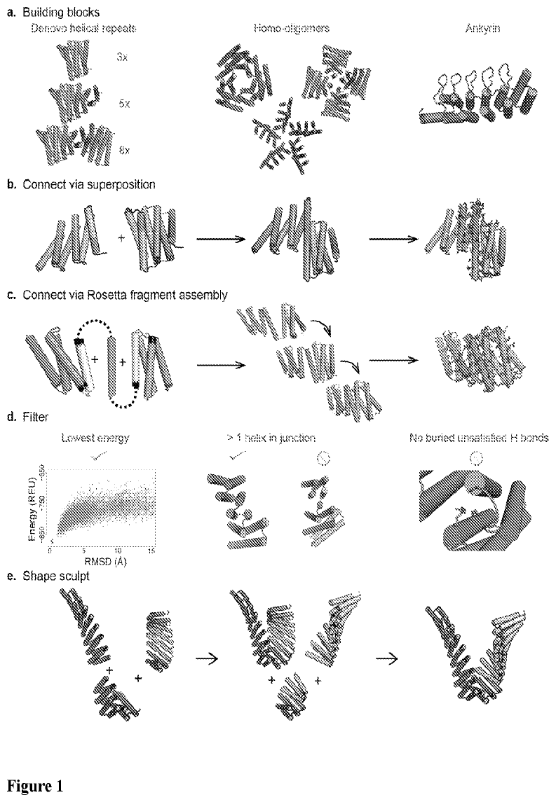

Here we describe a general approach for robustly joining together de novo designed repeat to generate a wide range of shapes. We apply the method to rigidly combine DHRs, designed homo-oligomers and DHR-Ankyrin fusions (FIG. 1a), and demonstrate that the junctions enable the specification of protein shape on the multiple nanometer length scale.

### Results

### Protein Fusion Approach

We set out to develop methods for systematically generating large sets of rigid protein building blocks by combinatorially fusing DHRs. We explored two approaches, the first based on helical superposition and the second on Rosetta™ fragment assembly. The helical superposition approach utilizes structure fusion through overlap of helical segments in our approach 6-residue helical segments in a first DHR are superimposed onto a 6-residue helical segment of a second DHR and the sequences of residues adjacent to the junction are optimized using RosettaDesign™ (FIG. 1b). We then select out the small fraction of the fusions in which the joined DHRs are in contact beyond the superimposed junction helix to reduce flexibility across the junction by requiring that at least two helices from each DHR make contact across the new interface. We also filtered out models with buried unsatisfied hydrogen bonds, and then used Rosetta™ de novo structure prediction calculations to identify sequences strongly specifying the designed structures in silico (FIG. 1d). With the helix fusion approach, we were able to generate an average of 2.7 junctions per DHR-DHR pair with sequences predicted to robustly fold into the designed shape in silico.

### Rosetta™ Fragment Assembly Approach:

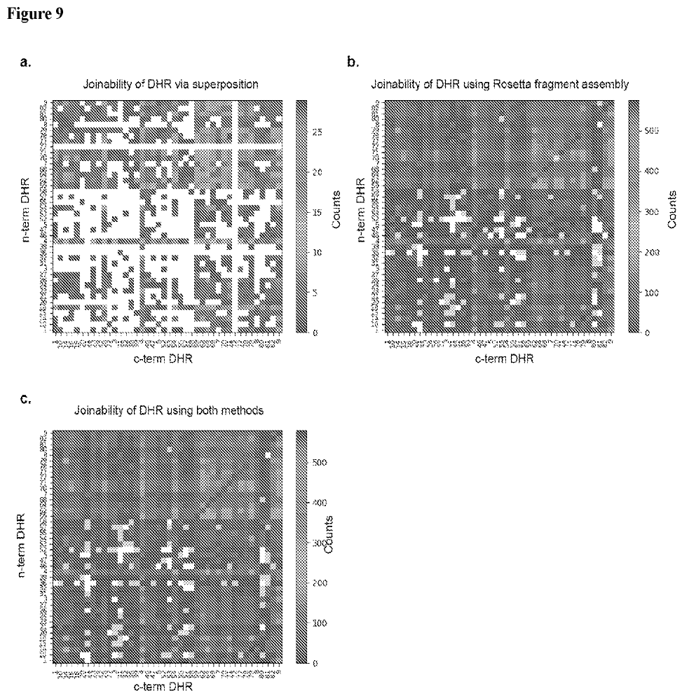

To access a larger number of junctions for a given repeat protein pair, we developed a Rosetta™ Monte Carlo fragment assembly approach which generates additional backbone structure to rigidly connect two DHRs. For each DHR pair, a new structural element was built to interface between the two domains, consisting of either a loop, a helix (with two loops) or two helices (with three loops). The lengths of the helices ranged from one less than the shortest of the helices in the DHR's being joined to one residue longer than the longest of the helices, and the length of the loops ranged from 2 to 4 residues (the total length of the inserted structure ranged from 2 to 64 residues). For each junction, we exhaustively generated all secondary structure strings (“blueprints”) consistent with these rules, and then built up backbone coordinates for each string through 3200 Monte Carlo fragment assembly steps. Following each fragment insertion, the net rigid body transform was propagated to the downstream repeat protein domain (FIG. 1c steps 1, 2 and SI Discussion S1); during this process the backbone in the flanking repeat proteins were kept rigid. Rosetta™ design was then used to design the amino acid sequence of the new residues and residues in the DHR that neighbor the new residues (FIG. 1c step 3). The same filters used in the helix superposition approach were applied to eliminate implausible and flexible structures. With the fragment assembly approach we were able to design an average of 40 junctions per DHR-DHR pair and connect almost all pairs of DHRs (FIG. 9).

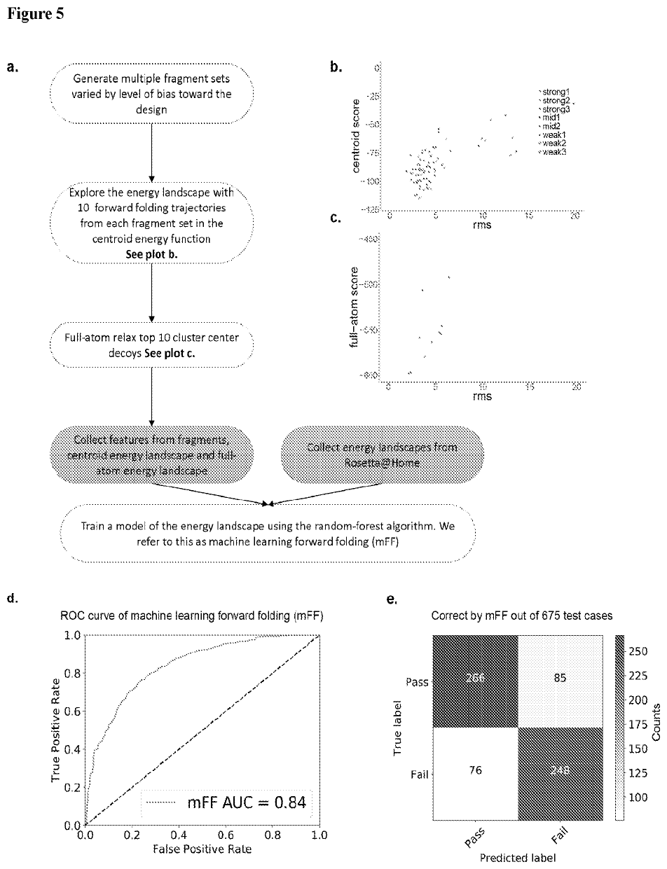

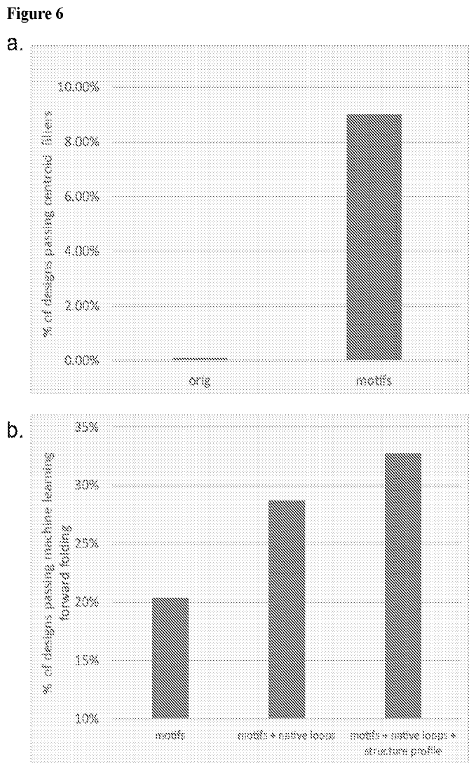

To make the large scale building of junction insertion regions between all pairs of repeat proteins computationally tractable, we increased the efficiency of the fragment assembly part of the second approach using several new algorithms which resulted in designs more similar to native structures in their core sidechain packing and turn geometry. First, the centroid backbone stage was biased toward native-like hydrophobic packing arrangements using the residue-pair transform (RPX) motif score, which favors residue-residue rigid body transforms observed between isoleucine, leucine, valine, and phenylalanine in the PDB. Incorporation of RPX motifs during low resolution backbone sampling increases the downstream yield of well packed designs 100 fold (FIG. 6. a). Second, we increased the quality of the local geometry in the junction regions, eliminating highly kinked helices and strained loops. Designs containing such structures fail the computationally expensive step of Rosetta™ de novo structure prediction, so it is advantageous to eliminate such local strain before structure prediction. To accomplish this we developed a new approach to filter out kinked helices and a loop closure technique that ensures every newly designed loop is within 0.4 RMSD of a frequently seen loop in the PDB (SI Discussion S1 step 4 and 5). Third, we developed an efficient approach to bias sequence design using a sequence profile generated from protein fragments with a similar structure to the design (SI Discussion S1 step 6). Together, the improvements in loop building and sequence design resulted in a 12% increase in the number of designs passing the final in silico validation by de novo structure prediction (FIG. 6b). Finally, we improved the efficiency of this last evaluation step by developing a protocol that predicts the results of large numbers of de novo folding simulations (carried out here on Rosetta@Home) using features from a small number of de-novo folding trajectories. These trajectories were biased by varying amounts toward the design model to sample both near the target structure and more broadly to allow more efficient estimation of the energy gap between the design and possible structurally divergent low energy states. This method recapitulates the results obtained with unbiased folding trajectories with 100 fold lower computational cost (FIG. 5 and SI Discussion S2).

### Experimental Characterization:

Using the design and filtering methods described above, followed by clustering with a 1A backbone rmsd threshold, we generated a set of 75 thousand designs that pass the in silico filtering metrics as well or better than their component DHRs (SI Discussion S5). 94% of these designs were generated with the Rosetta fragment assembly approach which explores more orientations between the DHRs and hence produces more solutions. we focused our experimental characterization on designs made using the Rosetta fragment assembly approach.

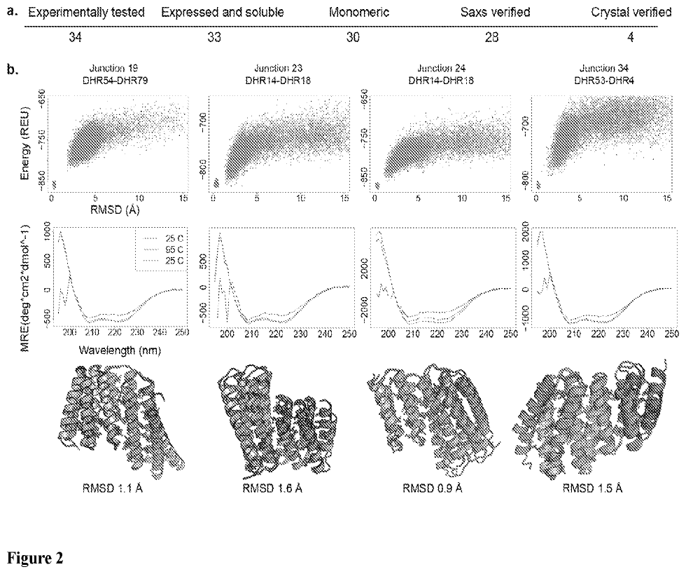

We obtained synthetic genes encoding a diverse set of 34 designs, expressed the proteins in E. coli and purified them by nickel NTA chromatography. 33 of 34 of the designs were soluble and had the expected alpha helical CD spectrum at 25° C., and 28 of the 34 were folded at 95° C. 30 of these proteins were monomeric as measured by analytical size exclusion chromatography coupled to multi-angle light scattering (SEC-MALS) (FIG. 2a).

We solved the crystal structures of 4 junctions with resolutions between 1.8 to 2.4 Å. The designs closely match the crystal structure with Ca RMSDs ranging from 0.9 Å to 1.6 Å (FIG. 2b). All of the structures add two loops and a helix between two DHRs. The designs closely match the crystal structures in the junction region. Junction 19 has an RMSD of 1.2 Å and matches closely, 0.9 Å, over the middle 110 residues but deviates slightly (1.4 Å over the 76 residues of the N and C terminal repeats) due to movement in the terminal helices also observed in the crystal structures of the components DHR54 and DHR79. Junction 23 and 24 are formed from the same building blocks (DHR14 and DHR18), but Junction 24 takes a sharp turn at the connection while Junction 23 is relatively straight; this difference is recapitulated in the crystal structures, showing that the junction method can assemble quite different geometries from the same building blocks. The crystal structure of the N-terminal DHR14 repeats in Junction 24 better matches the original design (0.8 Å) than the crystal structures of DHR14 both in isolation (1.0 Å) and in Junction 23 (0.9 Å); because of this the overall crystal structure of Junction 24 is closer to the design model than that of Junction 23 (0.9 Å vs 1.6 Å). Junction 34 connects DHR53 to DHR4 with a slight twist at the junction; the crystal structure shows some deviation in the N and C terminal helices. See SI discussion S3 and table 3 for further crystal structure analysis.

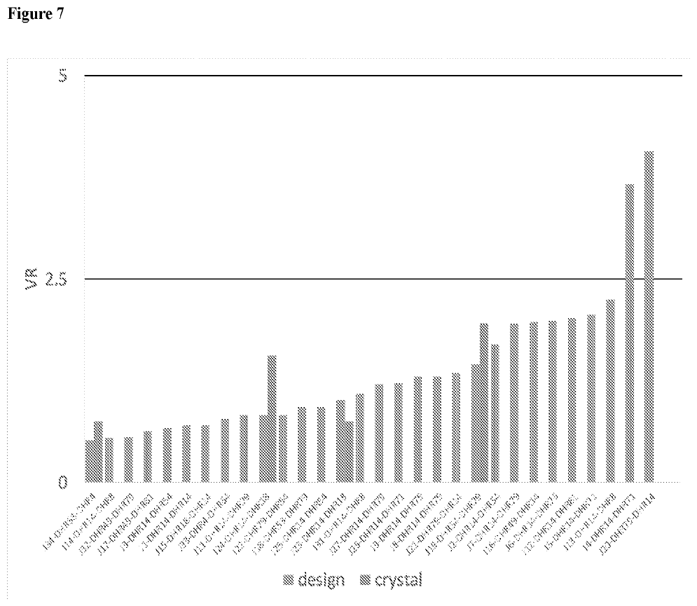

To characterize the overall shape of designs that did not crystallize we used solution x-ray scattering (SAXS). For 28 of the 30 monomeric proteins the radius of gyration (RG) and maximum distance (dmax) estimates obtained from the scattering profiles were close to those computed from the design models. We further compared the experimentally observed SAXs profiles with simulated profiles calculated from the corresponding design models using the volatility ratio (Vr) which has been shown to be more robust to noise (FIG. 7, SI Discussion S4). The maximum value of Vr obtained for the design models of the four junction crystal structures compared to the corresponding experimental SAXS spectra was 2.0, and amongst 15 previously determined crystal structures of DHRs, which have similar size and aspect ratio as the junctions, the maximum value was 2.5. Thus, designs with SAXS spectra matching spectra computed from the design models with Vr values less than 2.5 are likely be adopt structures close to the design models. 28 of the junction designs had Vr values below 2.5; the two proteins where the profiles did not match had dmax and RG approximately double that of the design indicating likely aggregation (Junction 4 and 20).

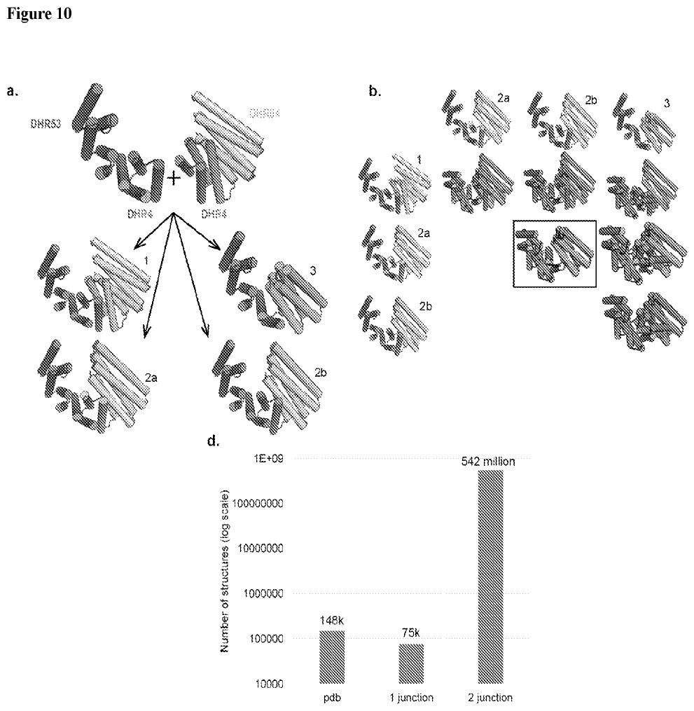

With this experimental validation of the capability of building rigid junctions, we generated a library of 75 thousand junctions between DHRs and 15 junctions between a DHR and a designed ankyrin¹⁷ built with the fragment assembly strategy. Any pair of these single junction proteins can be combined by matching a C-terminal and N-terminal DHR (FIG. 10 a). There are 542 million two junction combinations involving only DHRs, and billions when also including individual repeat proteins, homo-oligomers or ankyrin fusions (FIG. 10 and b). To facilitate generation and exploration of such multiple junction protein “sculpts”, we developed a parallelized python script that enumerates all DHR repeat lengths and junction combinations and writes a blueprint file which directs Rosetta to generate the three dimensional structures and sequences.

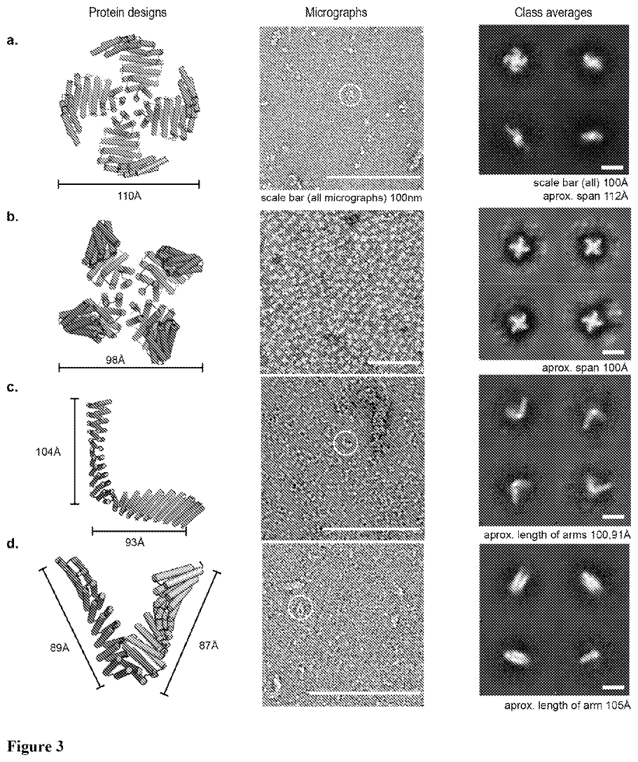

We used the enumerative method to generate large numbers of fused models, and selected two designs for experimental testing with ˜10 nm arms flanking the junction site(s) likely to be visible in negative stain electron microscopy (EM). The 975 residue “L” shape design is composed of one junction and the 853 residue “V” shape uses two junctions. To reduce possible recombination in synthetic genes encoding the designs, we introduced limited sequence variation in the surface helices of the structure. Both monomers expressed solubly in E coli and their structures, as assessed by negative stain EM, are in agreement with design models (FIG. 3). The “L” shape design links together 9 repeats of DHR14 and 9 repeats of DHR76 via a DHR14-DHR76 junction that produces a roughly 90 degree angle between the two arms. The individual repeat units of DHR14 and DHR76 are built from different length helices and the displacement along the repeat axis also differs; hence the longer arm, built from DHR14, is thinner than the shorter arm. The overall shape, the junction angle, and the differences in the thicknesses and lengths of the two arms are evident in the negative stain EM, both in the raw micrographs and in two-dimensional class averages (FIG. 3c; the shorter 93 Å arm is noticeably wider than the longer and thinner 104 Å arm). The “V” shape links together 7 repeats of DHR14 to 7 repeats of DHR54 via a DHR14-DHR79 and a DHR79-DHR54 junction. The negative stain 2D averages again are similar to the design model, with a close to “V” shape and with the two arms having similar widths and lengths. These results show that the junctions are sufficiently rigid to produce designs at the nanometer length scale.

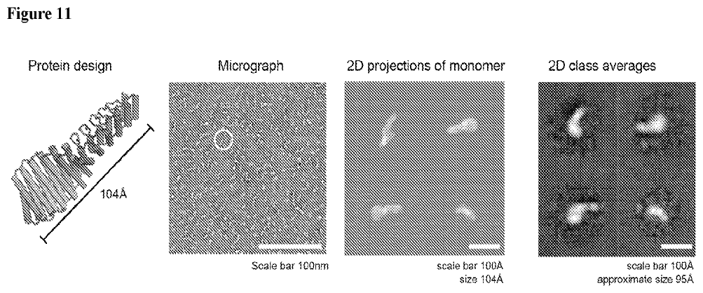

A potential application of the design methodology developed herein is to place receptor binding domains in relative orientations appropriate for engaging with multiple cell surface receptor subunits. To test our repeat protein junctions in the context of homo-oligomers, we generated junctions to four previously verified DHR-based oligomers that ranged in symmetry from C2-05. For each oligomer we generated 2-3 junction fusions that were at least 10 nm across to facilitate visualization in negative stain electron microscopy. Of the designs, 2 had negative stain EM images consistent with the design model. The spiral and X designs connect DHR53 to the HRO4C4_1 oligomer via a junction between DHR53 to DHR4 (FIG. 3 a,b). The spiral design has two more DHR4 repeats than the X shape, which flip the arms of the spiral up and into a claw like shape. A designed Ankyrin-DHR-C2 fusion disassociated in negative stain, but the monomer has a distinctive shape recapitulated in negative stain 2d-averages (FIG. 11) with a DHR component wider and shorter than the ankyrin component. SAXs data suggests the ankyrin-C2 is a dimer at the concentrations used in the scattering experiments as the experimental radius of gyration of 55 is closer to the dimer Rg of 49 than the monomer Rg of 35. All 3 designs validated by EM had SAXs distance distributions (dmax) and radius of gyration (Rg) consistent with the design (FIG. 7c). 5 of the designs that we were unable to validate by EM had SAXs dmax and Rg values that differed from the design by more than 25%. The Vr values of the EM validated designs range from 2.5-6.6 suggesting they are more flexible than the junction building blocks which all had Vr<2.5 (the Vr discrepancy also derives in part from the differences in sample size; the oligomer sculpt constructs are 10 nm across while the individual junctions span 4 nm or less).

### Discussion

The design methods described herein enable the rapid and accurate design of new proteins by fusing de novo designed repeat proteins. Of the 34 experimentally characterized single junction designs, 28 were close to the design model. The improvements in the efficiency and speed of the design protocol enabled the generation of 75 thousand junctions strongly predicted to have the designed structure. These improvements in computational efficiency will enable more research groups to design de novo proteins without the need for extensive computational resources and facilitate the design of increasingly complex structures.

Modern manufacturing was revolutionized by parts that could be used interchangeably and easily connected to one another. Here we begin to apply this concept to de novo proteins. More generally, the parts library developed here enables rapid exploration of applications to imaging and cell signaling. In contrast to approaches to joining domains with flexible linkers and bispecific antibodies, with the flexible hinge between the Fc and Fab, our junction library enables precise control over the orientation of the fused domains. This is important for both design of higher order protein assemblies and the arraying of receptor binding domains in precise orientations to engage cell surface receptors in predefined geometries. Our junction library makes the exploration of these and other applications limited not by the design of the monomers and assemblies, but the creativity of the protein engineers deploying the methods.

### REFERENCES

1. Hong, F., Zhang, F., Liu, Y. & Yan, H. DNA Origami: Scaffolds for Creating Higher Order Structures. Chem. Rev. 117, 12584-12640 (2017).
2. Jacobs, T. M. et al. Design of structurally distinct proteins using strategies inspired by evolution. Science 352, 687-690 (2016).
3. Glover, D. J., Giger, L., Kim, S. S., Naik, R. R. & Clark, D. S. Geometrical assembly of ultrastable protein templates for nanomaterials. Nat. Commun. 7, 11771 (2016).
4. Lai, Y.-T. et al. Designing and defining dynamic protein cage nanoassemblies in solution. Sci Adv 2, e1501855 (2016).
5. Youn, S.-J. et al. Construction of novel repeat proteins with rigid and predictable structures using a shared helix method. Sci. Rep. 7, 2595 (2017).
6. Parmeggiani, F. & Huang, P.-S. Designing repeat proteins: a modular approach to protein design. Curr. Opin. Struct. Biol. 45, 116-123 (2017).
7. Geiger-Schuller, K. et al. Extreme stability in de novo-designed repeat arrays is determined by unusually stable short-range interactions. Proc. Natl. Acad. Sci. U.S.A. 115, 7539-7544 (2018).
8. Brunette, T. J. et al. Exploring the repeat protein universe through computational protein design. Nature 528, 580-584 (2015).
9. Fallas, J. A. et al. Computational design of self-assembling cyclic protein homo-oligomers. Nat. Chem. 9, 353-360 (2017).
10. Shen, H. et al. De novo design of self-assembling helical protein filaments. Science 362, 705-709 (2018).
11. Pyles, H., Zhang, S., De Yoreo, J. J. & Baker, D. Controlling protein assembly on inorganic crystals through designed protein interfaces. Nature 571, 251-256 (2019).
12. Foight, G. W. et al. Multi-input chemical control of protein dimerization for programming graded cellular responses. Nat. Biotechnol. (2019) doi:10.1038/s41587-019-0242-8.
13. Maguire, J. B., Boyken, S. E., Baker, D. & Kuhlman, B. Rapid Sampling of Hydrogen Bond Networks for Computational Protein Design. J. Chem. Theory Comput. 14, 2751-2760 (2018).
14. Hura, G. L. et al. Robust, high-throughput solution structural analyses by small angle X-ray scattering (SAXS). Nat. Methods 6, 606-612 (2009).
15. Rambo, R. P. & Tainer, J. A. Super-resolution in solution X-ray scattering and its applications to structural systems biology. Annu. Rev. Biophys. 42, 415-441 (2013).
16. Hura, G. L. et al. Comprehensive macromolecular conformations mapped by quantitative SAXS analyses. Nat. Methods 10, 453-454 (2013).
17. Parmeggiani, F. et al. A general computational approach for repeat protein design. J. Mol. Biol. 427, 563-575 (2015).
18. Yeh, C.-T., Brunette, T. J., Baker, D., McIntosh-Smith, S. & Parmeggiani, F. Elfin: An algorithm for the computational design of custom three-dimensional structures from modular repeat protein building blocks. J. Struct. Biol. 201, 100-107 (2018).
19. Labrijn, A. F., Janmaat, M. L., Reichert, J. M. & Parren, P. W. H. I. Bispecific antibodies: a mechanistic review of the pipeline. Nat. Rev. Drug Discov. 18, 585-608 (2019).
20. Mohan, K. et al. Topological control of cytokine receptor signaling induces differential effects in hematopoiesis. Science 364, (2019).

### Supplemental Information:

### Discussion S1|Computational Protocol

### Overview

We developed two methods to rigidly fuse proteins together and used them to connect 44 designed helical repeat proteins (DHRs) into a building block library of 75k junctions, each with a unique shape. Proteins from this library were then used to sculpt larger, nanometer length proteins.

### A1. The Superposition Algorithm

In our approach to fuse two DHRs along a shared helix, six-residue helical segments from a first DHR were superimposed onto six-residue helical segments from a second DHR. A single repeat from each DHR was scanned. For overlaps less than 0.3 Å RMSD that did not clash, the sequence was redesigned for positions within 6 Å of the new DHR-DHR interface. Repack of side chains occurred for residues within 8 Å. Residues on the terminal DHR repeat were not redesigned. During design, surface residues were restricted to hydrophilic and core residues to hydrophobic by a Rosetta™ layer design task operator. After design, the structures were filtered according to step B.

### A2. The Rosetta™ Fragment Assembly Algorithm

A second way to make a rigid connection was to create additional residues between the two proteins using Rosetta™ fragment assembly. This proceeded in six steps:

### 1. Create Various DHR Trims

To explore a wide spectrum of possible junction geometries, the terminal helices were trimmed by one to four residues, which is enough to span one turn of an alpha-helix. For DHR combinations that were unable to be joined due to the filters applied in step B, additional interface geometries were explored such as trimming one helix out of the two-helix repeat; to keep these additional geometries compatible with the building block library, two terminal repeats were maintained.

### 2. Backbone Design Using Rosetta Fragment Assembly Guided by Motifs

For each DHR pair, additional amino acid residues were added using Rosetta fragment assembly between the two domains consisting of either a loop, a helix (with two loops), or two helices (with three loops). The lengths of the helices ranged from one less than the shortest helices of the DHRs being joined to one residue longer than the longest residue, and loops ranged from two to four residues. For structures with two helices, the helix length was restricted to be within one residue of the lengths of the DHR helices. All secondary structure possibilities consistent with these rules were exhaustively generated. Backbone coordinates were built up through 3,200 Monte Carlo fragment assembly steps with fragments harvested from a non-redundant set of structures from the PDB (1) starting from a structure with ideal helices and extended loops. Following each fragment insertion, the rigid body transform was propagated to the downstream repeat protein domain and the backbone in the flanking terminal repeat of the DHRs were kept rigid. The score that guided fragment assembly considers Van der Waal interactions, packing, backbone dihedrals angles and, for the first time, Residue-Pair-Transform (RPX) motifs (2). RPX motifs indicate when a portion of the backbone will pack together with hydrophobic residues in full-atom prior to assigning side chains (centroid representation). In this way, RPX motifs increase the accuracy of the centroid energy function.

### 3. Filter Backbones to Reduce Flexibility

To reduce flexibility across the junction, we require that at least two helices from each DHR and/or junction make contact across the new interface. We found that if a helix interacts with three or fewer other helices that structure had flexible point made up of a single helix. To determine which helices were in contact the Residue Pair Motifs (RPX) (2) was used. Structures with three helices in contact at the centroid stage can become four helices during the subsequent full-atom relax; as such, structures with <3 helices in contact were filtered.

4. Filter Backbones with Structural Features Dissimilar to Those in Solved Protein Structures

The validation step most likely to reject a design is Rosetta ab initio structure prediction. Since sequence design and filtering are computationally expensive steps, it is important to quickly triage structures that would fail ab initio. Designs are more likely to fail structure prediction when parts of the design do not resemble natural proteins. To explore the foldability of designs, nine residue fragments from the design were compared to all nine-residue fragments in the PDB. Proteins were more likely to pass Rosetta ab initio if the loops are within 0.4 Å RMSD and helices are within 0.14 Å RMSD to a structure in the PDB. A helix that is above 0.14 Å relative to all helices in the PDB appeared bent or kinked. All structures analyzed were helical with short (2-4 residue) loops so different values may be required when applying this filter to proteins with longer loops or sheets.

The algorithm to identify the most similar fragment took approximately one second to search through the four million fragments in the VALL PDB database (3). To achieve this speed, only fragments with the same secondary structure were compared, and RMSD was calculated using the Quaternion Characteristic Polynomial method (QCP kernel) (4, 5).

5. Fix Loops so they are Structurally Similar to Those in the PDB

A loop dissimilar to all loops in the PDB can often be repaired by swapping the designed loop with one from the PDB that better superimposes onto the end points of helices being bridged. To identify the loop that best matches onto the helix endpoints the two helical residues on either side of all short loops from the VALL pdb database were superimposed onto two stub residues at the end of the bridged helices. The four residue match with the lowest RMSD was considered the best match. To address small deviations in the overlapped residues the loop backbone was minimized after being placed by superposition. To explore a wide possibility of helical end point geometries the helices were extended and shrunk by three residues. The final loop RMSD was measured using the algorithm from step 4. Structures with loops >0.4 Å RMSD after fixing were filtered.

### 6. Sequence Design

Rosetta™ design was used to design the amino acid sequence of residues in the junction and residues in the repeat that neighbors the junction. Surface residues were restricted to hydrophilic and core residues to hydrophobic by a Rosetta™ layer design task operator. Sequence was further optimized to satisfy buried hydrogen bonds, match secondary structure predicted from sequence (psipred), and bias the sequence toward protein fragments with similar structure. The unsatisfied hydrogen bonds (6) and PSIPRED (7) sequence match were optimized using the generic simulated annealing mover in Rosetta™ which applies a Monte Carlo search over sequence design.

Sequence composition was biased toward native protein fragments with similar local structure using a structure profile. The structural profile used the fragment lookback approach described in step 4 to identify the most structurally similar nine residue fragments where the RMSD to the design was lower than 0.4 Å. Previously, structure profile generation would take 10-20 minutes and require a script outside of Rosetta™. Using the fragment lookback approach the structural profile now takes seconds to build.

### B. Filter

The junction library generated in the previous steps was filtered to ensure all proteins were of high quality and can be used to sculpt larger proteins. The proteins were filtered for uniqueness to 1.0 Å RMSD, lack of unsatisfied hydrogen bonds, a large and broad hydrophobic interface across multiple helices, and to have the lowest energy compared to other potential folds as measured by Rosetta™ ab initio. Most of these filter steps can be run on millions of proteins, but evaluating if the designed protein was in a lower energy state than alternative conformations can take several days on hundreds of CPUs using Rosetta ab initio. To speed up Rosetta™ ab initio, machine learning was used to simulate ab initio on a single CPU in 3-4 hours with high accuracy. The Rosetta™ ab initio step is described in more detail in SI Discussion 2.

### C. Sculpt

For protein sculpting, possible junction combinations containing one or two junctions were enumerated. The junction combinations were stored in a blueprint file that contains the information necessary for Rosetta to build protein sculpts. Due to the huge number of possible junction combinations, only a small and random subset of the possibilities were made. Ordering was done by visual inspection and designs that clash were discarded. For symmetric designs, symmetry was applied after the monomer construction.

Large proteins composed of numerous repetitive amino acid stretches require genes that are difficult to synthesize. To alleviate this problem the surface residues of all helices not part of the symmetric interface were redesigned using Rosetta™.

Discussion S2|Machine Learning Forward Folding (mFF)

In ab initio structure prediction (also called forward folding), the energy landscape is explored using short simulations starting from an initial extended structure (decoy). In each step of the simulation, a 9 or 3 residue fragment from a solved protein structure is swapped into the decoy and accepted using the Metropolis Monte Carlo criteria. Each simulation results in a decoy with an energy and distance from the design measured as root mean square deviation (RMSD). The design is validated if the distribution of decoys produces a funnel to the low energy and low RMSD designs. Thousands of decoys are required to suggest a design is lower in energy than alternative minima. To generate those decoys, Rosetta@Home is used to distribute the job to hundreds of users. A Rosetta@Home ab initio simulation can take several days, with a max throughput of 500-1000 simulations per week.

Ab initio validation contains more information than ab initio structure prediction, because structural prediction lacks the structural design data. Using information from the design can be used to bias exploration toward the design or not used so exploration broadly explores the entire energy landscape. To control this bias, 8 fragment sets were created that are subsets of the 200 fragments normally used in Rosetta™ ab initio. The 8 fragment sets used are listed with decreasing bias: top 3 by RMSD to design, top 15 by RMSD, from the first 25 fragments select the top 3, the top 3 plus a random 10 from 200, top 15 plus random 10, top 3 plus random 15, top 3 plus random 25, from the first 25 select a random 15. The top 200 fragments are ranked during fragment picking so fragments in the top 25 are more likely to be correct.

Using these 8 fragment sets ranging from strongly to weakly biased 10 centroid ab initio simulations were run. These 80 decoys were clustered and the low-energy cluster center is relaxed into the Rosetta™ full-atom energy function. It has been previously established that compute time can be saved by running full-atom Rosetta™ only on cluster centers (10).

Each of these eight centroids and one full atom simulations produces features that indicate if a protein would pass Rosetta™ ab initio structure prediction. These features are used to train a random forest that can predict if the protein design would pass ab initio structure prediction.

The features used are the lowest rms structure, the score range between structures, the standard deviation in RMSD between structures and average RMSD to the design. Additional features are extracted from the fragment sets including the percentage of fragments lower than 0.5, 1 and 1.5 Å RMSD and the average fragment quality for the top 3 and top 15 fragments sets.

To train the model we collected 2250 ab initio simulations on Rosetta@Home split evenly between cases that pass ab initio and those that did not. The simulations were labeled as passing ab initio if the ff_metric value is <25. FF_metric is an algorithm that uses the sum of RMSD in the lowest energy points to evaluate the funnel (11).

30% of the Rosetta@home simulations were set aside for testing and 70% used to train the model. The resulting random forest model had an AUC of 0.84 with error split between false positives, and false negatives. The top three features in the model are the low RMSD structure generated from the top 3, top 15 and top 3 plus 25 fragment sets.

Machine learning forward folding (mFF) takes about 3-4 hours on a single core as compared to several days on hundreds of user computers. This dramatic speed improvement allows us to simulate thousands of de novo protein designs when previously we could only simulate hundreds. It also allows us to screen designs before submitting to Rosetta@Home.

### Discussion S3|Crystal Structure Determination Analysis

Junction 19 is between DHR54 and DHR79 and had an RMSD of 1.14 Å to the crystal structure. The main deviation between the design and crystal is observed in the c-terminal helix, the likely result of a crystal-packing artifact. The n-terminal repeat and the core rotamers are in their designed positions.

Junction 23 is between DHR14 and DHR18 and had an RMSD of 1.58 Å to chain A of the crystal structure. We observed a slight deviation in the n-terminal repeat structure relative to the design. It appears that the n-terminal repeat twist does not occur in the junction itself but in the second repeat past the junction. There is a second chain resolved in the crystal structure, with an RMSD deviation of 1.5 Å relative to the design. The N-terminal helix is not resolved in the structure and is presumed to be disordered.

Junction 24 is between DHR14 and DHR18 had an RMSD of 0.93 Å relative to the crystal structure. A 5-residue stretch in the c-terminal portion of the protein is disordered. Disorder of the c-terminal helix previously occurred to several of the DHR proteins (8).

The design of junction 31 between DH53 and DHR4 had an RMSD of 1.51 Å to the crystal structure. There appears to be a slight twist in the junction.

TABLE 3
Crystallographic data collection and refinement statistics

| | Junction 19 | | Junction 23 | | Junction 24 | | Junction 34 | |
|---|---|---|---|---|---|---|---|---|
| | DHR54-DHR79 | | DHR14-DHR18 | | DHR14-DHR18 | | DHR53-DHR4 | |
| Wavelength | 0.9999 | | 0.9791 | | 1 | | 1 | |
| Resolution range | 45.63-2.35 | | 33.89-2.40 | | 43.71-2.21 | | 37.46-1.8 | |
| | (2.43-2.35) | | (2.49-2.40) | | (2.29-2.21) | | (1.86-1.80) | |
| Space group | P 1 21 1 | | P 1 21 1 | | P 21 21 21 | | P 21 21 21 | |
| Unit cell | 53.7 109.7 81.0 | | 62.0 41.1 94.0 | | 49.1 49.7 92.0 | | 43.9 57.6 71.7 | |
| | 90 107.5 90 | | 90 104.9 90 | | 90 90 90 | | 90 90 90 | |
| Total reflections | 219869 | (22021) | 198525 | (8047) | 69268 | (6908) | 117427 | (11054) |
| Unique reflections | 37152 | (3256) | 17440 | (1307) | 11787 | (1147) | 17375 | (1692) |
| Multiplicity | 5.9 | (6.0) | 11.4 | (6.0) | 5.9 | (6.0) | 6.8 | (6.5) |
| Completeness (%) | 87.01 | (63.89) | 88.97 | (62.88) | 95.32 | (85.93) | 94.30 | (80.32) |
| Mean I/sigma(I) | 15.26 | (1.32) | 11.23 | (2.39) | 18.59 | (2.05) | 13.06 | (1.06) |
| Wilson B-factor | 48.6 | | 69.6 | | 43.6 | | 30.8 | |
| R-merge | 0.08 | (1.76) | 0.11 | (0.57) | 0.06 | (0.94) | 0.07 | (1.31) |
| R-meas | 0.09 | (1.93) | 0.13 | (0.61) | 0.07 | (1.03) | 0.07 | (1.42) |
| R-pim | 0.0367 | (0.78) | 0.0332 | (0.22) | 0.028 | (0.42) | 0.028 | (0.55) |
| CC1/2 | 1 | (0.57) | 0.997 | (0.87) | 0.999 | (0.7) | 0.999 | (0.54) |
| CC* | 1 | (0.85) | 0.999 | (0.97) | 1 | (0.91) | 1 | (0.84) |
| Reflections used | 32655 | (2385) | 16292 | (1113) | 11244 | (989) | 16462 | (1371) |
| in refinement | | | | | | | | |
| Reflections used | 1794 | (133) | 1643 | (99) | 1110 | (101) | 1648 | (131) |
| for R-free | | | | | | | | |
| R-work | 0.24 | (0.38) | 0.24 | (0.33) | 0.23 | (0.32) | 0.20 | (0.30) |
| R-free | 0.27 | (0.39) | 0.27 | (0.36) | 0.25 | (0.35) | 0.23 | (0.33) |
| CC(work) | 0.97 | (0.71) | 0.95 | (0.80) | 0.97 | (0.75) | 0.96 | (0.74) |
| CC(free) | 0.95 | (0.62) | 0.95 | (0.71) | 0.96 | (0.66) | 0.95 | (0.66) |
| Number of non- | 5715 | | 2763 | | 1537 | | 1533 | |
| hydrogen atoms | | | | | | | | |
| macromolecules | 5701 | | 2762 | | 1517 | | 1435 | |
| ligands | | | | | | | 1 | |
| solvent | 14 | | 1 | | 20 | | 97 | |
| Resolution range | 45.63-2.35 | | 33.89-2.40 | | 43.71-2.21 | | 37.46-1.8 | |
| | (2.43-2.35) | | (2.49-2.40) | | (2.29-2.21) | | (1.86-1.80) | |
| Space group | P 1 21 1 | | P 1 21 1 | | P 21 21 21 | | P 21 21 21 | |
| Unit cell | 53.7 109.7 81.0 | | 62.0 41.1 94.0 | | 49.1 49.7 92.0 | | 43.9 57.6 71.7 | |
| | 90 107.5 90 | | 90 104.9 90 | | 90 90 90 | | 90 90 90 | |
| Total reflections | 219869 | (22021) | 198525 | (8047) | 69268 | (6908) | 117427 | (11054) |
| Unique reflections | 37152 | (3256) | 17440 | (1307) | 11787 | (1147) | 17375 | (1692) |
| Multiplicity | 5.9 | (6.0) | 11.4 | (6.0) | 5.9 | (6.0) | 6.8 | (6.5) |
| Completeness (%) | 87.01 | (63.89) | 88.97 | (62.88) | 95.32 | (85.93) | 94.30 | (80.32) |
| Mean I/sigma(I) | 15.26 | (1.32) | 11.23 | (2.39) | 18.59 | (2.05) | 13.06 | (1.06) |
| Wilson B-factor | 48.6 | | 69.6 | | 43.6 | | 30.8 | |
| R-merge | 0.08 | (1.76) | 0.11 | (0.57) | 0.06 | (0.94) | 0.07 | (1.31) |
| R-meas | 0.09 | (1.93) | 0.13 | (0.61) | 0.07 | (1.03) | 0.07 | (1.42) |
| R-pim | 0.0367 | (0.78) | 0.0332 | (0.22) | 0.028 | (0.42) | 0.028 | (0.55) |
| CC1/2 | 1 | (0.57) | 0.997 | (0.87) | 0.999 | (0.7) | 0.999 | (0.54) |
| CC* | 1 | (0.85) | 0.999 | (0.97) | 1 | (0.91) | 1 | (0.84) |
| Reflections used | 32655 | (2385) | 16292 | (1113) | 11244 | (989) | 16462 | (1371) |
| in refinement | | | | | | | | |
| Reflections used | 1794 | (133) | 1643 | (99) | 1110 | (101) | 1648 | (131) |
| for R-free | | | | | | | | |
| R-work | 0.24 | (0.38) | 0.24 | (0.33) | 0.23 | (0.32) | 0.20 | (0.30) |
| R-free | 0.27 | (0.39) | 0.27 | (0.36) | 0.25 | (0.35) | 0.23 | (0.33) |
| CC(work) | 0.97 | (0.71) | 0.95 | (0.80) | 0.97 | (0.75) | 0.96 | (0.74) |
| CC(free) | 0.95 | (0.62) | 0.95 | (0.71) | 0.96 | (0.66) | 0.95 | (0.66) |

| Number of non- | 5715 | 2763 | 1537 | 1533 |
|---|---|---|---|---|
| hydrogen atom* | | | | |
| macromolecules | 5701 | 2762 | 1517 | 1435 |
| ligands | | | | 1 |
| solvent | 14 | 1 | 20 | 97 |

Statistics for the highest-resolution shell are shown in parentheses.

### Discussion S4|Small Angle X-Ray Scattering (SAXS) Analysis

To characterize the structures of proteins we used Small Angle X-Ray Scattering (SAXS) analysis (12-15). with data collected at the SIBYLS beamline (16). Data frames were merged using the SAXS Frameslice™ program. The Porod, q range, Guinier, realspace p(r), model p(r) and crystal fit were solved using SCÅTTER 3.0 g (14). The model fit measurements of the volatility of ratio (Vr) and Chi were calculated using scripts from (15).

The protein designs and crystals were prepared for SAXs by adding missing residues and the n-terminal GWLEHHHHHH (SEQ ID NO:144) purification tag with Rosetta. The tag was added using Rosetta™ ab initio structure prediction on Rosetta@Home. The lowest energy 100 decoy were then clustered. Vr and chi were calculated for the top 5 cluster centers and the lowest VR was reported. Subsequent analysis within SCÅTTER was conducted using the design with the tag that produced the lowest Vr.

Data was collected on the 30 designs that were monomeric in SEC. The 28 designs with Vr<2.5 were considered successes. The 2.5 Vr cutoff was the maximum Vr of a design that produced a crystal structure (8). Additionally, all 30 designs had a Porod of >3.8 indicating a well-folded core. 27 of the designs had a Vr<2.5, and real space radius of gyration (Rg) and a maximum of distance distribution (dmax) within 30% of the model. For 1 design, junction 12, the Vr was <2.5 but the dmax was 38% of the model indicating there is likely aggregation.

The two failed proteins, Junction 4 and 20, had a Vr score greater than 2.5. These failed designs also had a dmax and Rg significantly higher than predicted indicating there was likely aggregation.

### Discussion S5|Filtering and Coverage of Junction Library

A key step in protein design is typically visual inspection to eliminate designs that appears good by Rosetta™ score metrics but poor by visual inspection. An example of this would be buried unsatisfied hydrogen bonds. The Rosetta™ metric for solvent accessible surface area (SASA) will evaluate a residue to be at the surface when the bond is close to a small pocket. While the protein designer may intuit that pocket is unlikely to exist so the hydrogen bond is unsatisfied in the core. The parameter to control pocket detection (SASA) could be tuned to match human intuition for that one case but in another case, a good design would be discarded.

For our filters, we attempted to identify thresholds that would allow all experimentally verified DHR to pass while filtering all designs that human intuition would discard. We were unable to identify a perfect filter threshold that would accomplish both goals. The filters we used are >1 helix in junction, no buried unsatisfied hydrogen bonds and that the design is the lowest point in the energy landscapes which was modeled with machine learning (mFF). For the filter thresholds that best matched human intuition 14 of the 44 experimentally verified DHRs would also be discarded; DHR53, 80 and 81 fail to have >1 helix in junction. DHR10, 52, 77, 78, 79 and 81 fail the unsatisfied hydrogen bond filter. And DHR1, 5, 10, 36, 46, 47, 53 and 59 fail mFF.

To allow DHRs to be joined where the DHR itself is below the filter cutoffs we relax the thresholds to require junctions be better than their component DHR. For >1 helix in a junction, the design must have more contact between neighboring helices than either component DHR. For unsatisfied hydrogen bonds, the junction must have fewer unsatisfied hydrogen bonds than the initial design. And for mFF, the junction must be more likely to fold than the average of the two parent DHRs. The resulting database of junctions contains 75k designs.

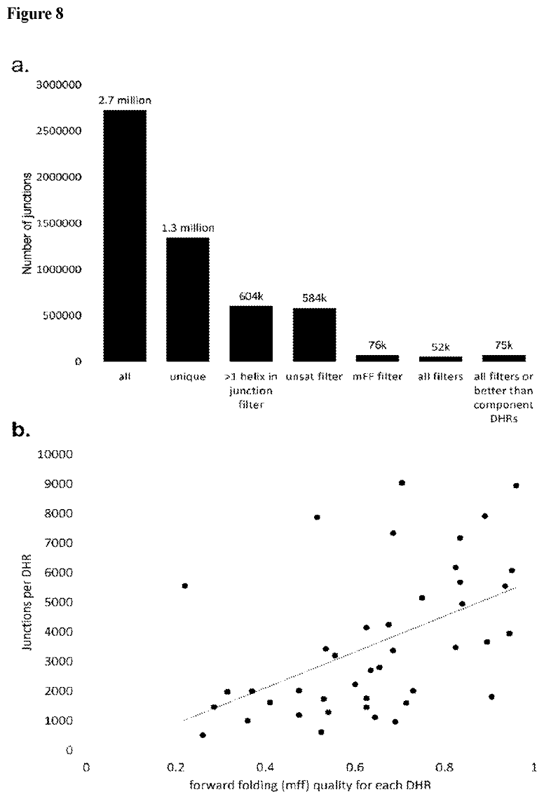

FIG. 8 a shows the number of designs filtered at each stage. The joinability between DHR correlates with the quality of mFF of the parent DHR (FIG. 8 b).

TABLE 4
Summary of sculpt data
a.

| | | | Correct | Correct | |
|---|---|---|---|---|---|
| | | Expressed | oligomeric state | Rg by | EM |
| | Tested | and soluble | by sec-mals | SAXs | verified |
| Monomer | 2 | 2 | 2 | 2 | 2 |
| sculpt | | | | | |
| Oligomer | 9 | 8 | 6 | 3 | 2, 1 |
| sculpt | | | | | as monomer |

b.

| | | | Correct | Correct | | |
|---|---|---|---|---|---|---|
| Oligomer | | Expressed | oligomeric state | Rg by | EM | |
| Component | Tested | and soluble | by sec-mals | SAXs | verified | Note |
| C2-(tj18C2_V03) | 2 | 2 | 2 | 1 | 1(as | dimer interface looks |
| | | | | | monomer) | correct in SEC-mals |
| | | | | | | and SAXs but not in |
| | | | | | | negative stain EM |
| C3-(tj41C3_pm1v2) | 2 | 2 | 1 | 0 | 0 | |
| C4-(HR04C4_1) | 2 | 2 | 2 | 2 | 2 | |
| C5-(HR10C5_2) | 3 | 3 | 2 | 0/1* | 0 | |

a. All monomer sculpts were able to be validated by EM but only 22% of oligomers were correct.
b. Oligomer success rate appears correlated to which oligomer is used. The C4 oligomer had a 100% success rate, while the C2, C3, and C5 oligomers fail more frequently.

### Discussion S6|Protein Sculpt Analysis

100% of the monomer sculpts had the correct shape by electron microscopy (EM). While only 22% of the oligomer sculpts were correct by EM. In most cases of EM failure, the SAXs Rg value does not match while the SEC mals size matches the correct oligomer. This suggests there may be re-arrangement happening at the interface or the interface is breaking. Also, all of the oligomer successes came from the same C4 building block. Future work will seek to identify the most stable oligomer building blocks or to design more robust building blocks. For details see Table 4.

### Sequences: See Tables 1 and 2

### Discussion S9|Methods for Expression, Crystallization, SAXs and Negative Stain Electron Microscopy

### Protein Expression and Characterization:

Genes were synthesized and cloned by IDT into pET29b. Genes were optimized for E. coli expression using DNAworks™ (17). For the 34 junction proteins, an addition c-terminal tag of GWLEHHHHHH (SEQ ID NO:144) was added; W was added for tracking protein concentration through absorbance at A280. For the protein “sculpts” the tag was changed to the n-terminal HHHHHHHGGS (His tag; SEQ ID NO:145), GENLYFQG (TEV site; SEQ ID NO:146), GSGWG (flexible region+W; SEQ ID NO:147), except for cases where the n-terminal was part of the dimer interface. In those cases, the original c-terminal tag was used. The genes for the 800+ residue protein “L” and “V” sculpts were synthesized by Genscript.

Proteins were expressed in E. coli Lemo21s using 500 μM isopropyl-β-D-thiogalactopyransoide (IPTG) after 4 hours at 37° C. in Terrific Broth (TB) growth medium. Cells were harvested by centrifugation and lysed using a Microfluidizer (Microfluidics) and purified by metal ion affinity (IMAC) and size-exclusion chromatography (SEC). The lysis buffer was 20 mM Tris pH 8.0, 500 mM NaCl, DNase, 0.25% CHAPS. The wash buffer was 20 mM Tris pH 8.0, 500 mM NaCl, 30 mM imidazole. The elution buffer was 20 mM Tris pH 8.0, 150 mM NaCl and 250 mM imidazole. Following the IMAC step, proteins were dialyzed in 20 mM Tris 150 mM NaCl pH 8.0. Protein concentrations were measured using a NanoDrop™ spectrophotometer (Thermo Scientific). Thermal denaturation and secondary structure content were monitored by circular dichroism (CD) using an AVIV 420 spectrometer (Aviv Biomedical). Oligomeric states were measured by analytical gel filtration (Superdex™ 75 or 200, GE Healthcare) coupled with multiple-angle light scattering (SEC-MALS). Molecular weights were confirmed by mass spectrometry on an LCQ Fleet™ Ion Trap Mass Spectrometer (Thermo Scientific).

### Crystallization:

All crystallization trials were carried out at 22° C. in 96-well format using the hanging-drop method. Crystal trays were set up using a Mosquito™ crystallization robot enclosed in a humidifying chamber (TTP labtech). Drop volumes ranged from 200 to 400 nl and contained protein to crystallization solution in ratios of 1:1, 2:1 and 1:2. All crystals were frozen in liquid nitrogen prior to shipment to the Advanced Light Source (ALS, Berkeley, Calif.) or the Advanced Photon Source (APS, Lemont, Ill.) for diffraction data collection. All datasets were integrated and scaled in HKL2000 (18). Diffraction data quality was assessed using Xtriage™ in the Phenix™ software suite (19). Phase information was obtained by molecular replacement in PHASER (20), using either the original Rosetta™ Design models or related low-energy variants as the search models. Initial models were automatically obtained using Phenix.autobuild (21). Final models were produced after iterative rounds of manual building in Coot (22) and refinement with Phenix.refine (23). Final resolution cutoffs were determined by monitoring the refinement statistics in the context of the reflection data completeness and the CC ½ values of the original diffraction data (24). The geometric quality of the final models was assessed using Molprobity™ (25).

Junction 19—Crystals were grown in Qiagen JCSG+ condition E5 (0.1M CAPS pH 10.5, 40% MPD) and required no additional cryopreservation. Diffraction data was collected on ALS beamline 8.2.2., 280 images with 1° increments.

Junction 23—Crystals were grown in Qiagen MPD condition A9 (0.2 Ammonium chloride, 40% MPD) and required no additional cryopreservation. Diffraction data were collected on APS beam line NE-CAT 24-ID-C, 1200 images with 0.25° oscillations.

Junction 24—Crystals were grown in Qiagen JCSG+ suite condition D9 (0.19M Ammonium sulfate, 25.5% (w/v) PEG 4000, 15% (v/v) glycerol) and required no additional cryopreservation. Diffraction data was collected on ALS beamline 8.2.2., 150 images with 1° oscillations.

Junction 34—Crystals were grown in Qiagen JCSG Core III suite condition G5 (0.2M calcium chloride dihydrate, 20% (w/v) PEG 3500. Crystals were briefly soaked in crystallization condition supplemented with 25% (v/v) PEG 400 as a cryoprotectant. Diffraction data was collected on ALS beamline 8.2.2., 200 images with 1° oscillations.

Data collection and refinement statistics are given in Table 3

### SAXS:

SAXs data was collected at the SIBYLS 12.3.1 beamline at the advanced light source LBNL (13, 16, 26) using the same method as used in (8). Data was averaged and sliced using the SAXs Frameslice program and analyzed using SCÅTTER 3.0 g program (14). An in-depth analysis of the SAXs method can be found in the supplementary information.

### Negative Stain Electron Microscopy

Samples were applied to glow-discharged continuous carbon film EM grids and stained with 1% uranyl formate. Designs that failed with the uranyl formate stain were tried with nano-tungsten stain but these still failed. Screens were run on an FEI Morgagni 268 electron microscope operating at an accelerating voltage of 100 kV. Grids were then examined using a Tecnai Spirit G2 transmission electron microscope operating at an acceleration voltage of 120 kV. Micrographs were acquired at a magnification of 67,000× and pixel size of 1.60 Å with a Gatan Ultrascan™ 4000 CCD via Leginon™ software (27). Approximately 100 micrographs were collected per sample at a defocus range between 1-1.5 μm. Image processing, including CTF estimation, particle picking, and 2D reference-free classification, was performed using the software package cisTEM (28). Multiple rounds of 2D classification were carried out to remove junk particles, and selected representative final averages are shown. The 2D projection images in FIG. S8 were generated using the v4 projection tool in the Eman 1.9 software package (29).

### SUPPLEMENTARY REFERENCES

1. C. A. Rohl, C. E. M. Strauss, K. M. S. Misura, D. Baker, Protein structure prediction using Rosetta. Methods Enzymol. 383, 66-93 (2004).
2. J. A. Fallas, et al., Computational design of self-assembling cyclic protein homo-oligomers. Nat. Chem. 9, 353-360 (2017).
3. P. Bradley, K. M. S. Misura, D. Baker, Toward high-resolution de novo structure prediction for small proteins. Science 309, 1868-1871 (2005).
4. D. L. Theobald, Rapid calculation of RMSDs using a quaternion-based characteristic polynomial. Acta Crystallogr. A 61, 478-480 (2005).
5. P. Liu, D. K. Agrafiotis, D. L. Theobald, Fast determination of the optimal rotational matrix for macromolecular superpositions. J. Comput. Chem. 31, 1561-1563 (2010).
6. J. B. Maguire, S. E. Boyken, D. Baker, B. Kuhlman, Rapid Sampling of Hydrogen Bond Networks for Computational Protein Design. J. Chem. Theory Comput. 14, 2751-2760 (2018).
7. D. T. Jones, Protein secondary structure prediction based on position-specific scoring matrices. J. Mol. Biol. 292, 195-202 (1999).
8. T. J. Brunette, et al., Exploring the repeat protein universe through computational protein design. Nature 528, 580-584 (2015).
9. R. Das, et al., Structure prediction for CASP7 targets using extensive all-atom refinement with Rosetta@home. Proteins 69 Suppl 8, 118-128 (2007).
10. T. J. Brunette, O. Brock, Guiding conformation space search with an all-atom energy potential. Proteins 73, 958-972 (2008).
11. G. J. Rocklin, et al., Global analysis of protein folding using massively parallel design, synthesis, and testing. Science 357, 168-175 (2017).
12. R. P. Rambo, J. A. Tainer, Super-resolution in solution X-ray scattering and its applications to structural systems biology. Annu. Rev. Biophys. 42, 415-441 (2013).
13. G. L. Hura, et al., Robust, high-throughput solution structural analyses by small angle X-ray scattering (SAXS). Nat. Methods 6, 606-612 (2009).
14. R. P. Rambo, J. A. Tainer, Accurate assessment of mass, models and resolution by small-angle scattering. Nature 496, 477-481 (2013).
15. G. L. Hura, et al., Comprehensive macromolecular conformations mapped by quantitative SAXS analyses. Nat. Methods 10, 453-454 (2013).
16. S. Classen, et al., Implementation and performance of SIBYLS: a dual endstation small-angle X-ray scattering and macromolecular crystallography beamline at the Advanced Light Source. J. Appl. Crystallogr. 46, 1-13 (2013).
17. D. M. Hoover, J. Lubkowski, DNAWorks: an automated method for designing oligonucleotides for PCR-based gene synthesis. Nucleic Acids Res. 30, e43 (2002).
18. Z. Otwinowski, W. Minor, Processing of X-ray diffraction data collected in oscillation mode. Methods Enzymol. 276, 307-326 (1997).
19. P. D. Adams, et al., PHENIX: a comprehensive Python-based system for macromolecular structure solution. Acta Crystallogr. D Biol. Crystallogr 66, 213-221 (2010).
20. A. J. McCoy, et al., Phaser crystallographic software. J. Appl. Crystallogr 40, 658-674 (2007).
21. T. C. Terwilliger, et al., Iterative model building, structure refinement and density modification with the PHENIX AutoBuild wizard. Acta Crystallogr. D Biol. Crystallogr. 64, 61-69 (2008).
22. P. Emsley, B. Lohkamp, W. G. Scott, K. Cowtan, Features and development of Coot. Acta Crystallogr. D Biol. Crystallogr. 66, 486-501 (2010).
23. P. V. Afonine, et al., Towards automated crystallographic structure refinement with phenix.refine. Acta Crystallogr. D Biol. Crystallogr. 68, 352-367 (2012).
24. P. A. Karplus, K. Diederichs, Linking crystallographic model and data quality. Science 336, 1030-1033 (2012).
25. V. B. Chen, et al., MolProbity: all-atom structure validation for macromolecular crystallography. Acta Crystallogr. D Biol. Crystallogr. 66, 12-21 (2010).
26. S. Classen, et al., Software for the high-throughput collection of SAXS data using an enhanced Blu-Ice/DCS control system. J. Synchrotron Radiat. 17, 774-781 (2010).
27. C. Suloway, et al., Automated molecular microscopy: the new Leginon system. J. Struct. Biol. 151, 41-60 (2005).
28. T. Grant, A. Rohou, N. Grigorieff, cisTEM, user-friendly software for single-particle image processing. Elife 7 (2018).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements. All headings are for the convenience of the reader and should not be used to limit the meaning of the text that follows the heading, unless so specified.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. A polypeptide comprising an amino acid sequence at least 50% identical to the amino acid sequence selected from SEQ ID NOS:1-78, wherein residues in parentheses are optional and may be present or absent.

2. The polypeptide of claim 1, comprising an amino acid sequence at least 80% identical to the amino acid sequence selected from SEQ ID NOS:1-78, wherein residues in parentheses are optional and may be present or absent.

3. The polypeptide of claim 1, comprising an amino acid sequence at least 90%, identical to the amino acid sequence selected from SEQ ID NOS:1-78, wherein residues in parentheses are optional and may be present or absent.

4. The polypeptide of claim 1, wherein mutations in hydrophobic residues relative to the reference sequence are conservative amino acid substitutions.

5. The polypeptide of claim 1, wherein mutations in residues relative to the reference sequence are conservative amino acid substitutions.

6. A fusion protein, comprising the general formula X1-X2-X3, wherein the fusion protein is selected from the following group, wherein residues in parentheses are optional and may be present or absent:
(a) X1 and X3 each independently is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86, wherein residues in parentheses are optional; and X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:1-2, wherein residues in parentheses are optional;
(b) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:3-4, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104;
(c) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:5-6, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104;
(d) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:7-8, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:113;
(e) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:9-10, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:113;
(f) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:11-12, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:115;
(g) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:13-14, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;
(h) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:15-16, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;
(i) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:17-18, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;
(j) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:19-20, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;
(k) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:21-22, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;
(l) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:23-24, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:120;
(m) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:25-26, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:83;
(n) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:27-28, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:83;
(o) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:88; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:29-30, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86;
(p) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:101; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:31-32, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86;
(q) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:101; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:33-34, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:120;
(r) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:35-36, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;
(s) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:37-38, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;
(t) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:39-40, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86;
(u) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:41-42, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86;
(v) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:43-44, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104;
(w) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:45-46, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:88;
(x) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:47-48, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:88;
(y) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:49-50, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:104;
(z) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:51-52, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:113;
(aa) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:53-54, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;
(bb) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:55-56, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;
(cc) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:57-58, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:83;
(dd) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:59-60, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:83;
(ee) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:86; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:61-62, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:83;
(ff) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:101; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:63-64, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:118;
(gg) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:80; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:65-66, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:110; and
(hh) X1 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:103; X2 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:67-68, wherein residues in parentheses are optional; and X3 is a DHR polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence of SEQ ID NO:80.

7. The fusion protein of claim 6, comprising the general formula X1-X2-X3-X₄, wherein X4 is a junction polypeptide comprising an amino acid sequence at least 50%, 55%, 60%, 65%, 70%, 75%, 80%, 85%, 90%, 91%, 92%, 93%, 94%, 95%, 96%, 97%, 98%, 99%, or 100% identical to a junction polypeptide that can be used to form a junction with X3 as shown in Table 1.

8. The fusion protein of claim 6, comprising the general formula X1-X2-X3-X4, wherein X4 is at least 50%, 55%, 60%, 65%, 70%, 75%, 80%, 85%, 90%, 91%, 92%, 93%, 94%, 95%, 96%, 97%, 98%, 99%, or 100% identical to the junction polypeptide of X2.

9. The fusion protein of claim 7, comprising the general formula X1-X2-X3-X4-X5, X5 comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to a DHR polypeptide that can be used with the X4 junction as shown in Table 1.

10. A polypeptide comprising an amino acid sequence at least 50% identical to the amino acid sequence selected from SEQ ID NOS:121-142, wherein residues in parentheses are optional and may be present or absent.

11. The polypeptide of claim 10, wherein mutations in hydrophobic residues relative to the reference sequence are conservative amino acid substitutions.

12. The polypeptide of claim 10, wherein mutations in residues relative to the reference sequence are conservative amino acid substitutions.

13.-16. (canceled)

17. A polymer comprising 2 or more copies of the fusion proteins of claim 6.

18.-20. (canceled)

21. A library comprising 5, 10, 25, 50, 75, 100, 250, 500, 1000, or more different polypeptides of claim 1.

22. A nucleic acid encoding the polypeptide of claim 1.

23. An expression vector comprising the nucleic acid of claim 22 operatively linked to a suitable control element.

24. A host cell comprising the expression vector of claim 23.

25.-37. (canceled)


SEQUENCE LISTING (147 sequences)

SEQ ID NO: 1 - protein, 211 aa, Artificial Sequence
  Synthetic peptide
MDSEEVNERVKQLAEKAKEATDKEEVIEIVKELAELAKQSTDPEVVKEIVTQLAQVAQESTNEELIREIIEVLKELLKEAQTPEEQAFIAAAIAAAAAKSGNEEEVRQAIQKAAELASQTSEESVKELVRELAELAKKAKDPKAVEAIVQLLAELAKKSSDSELVNEIVKQLEEVAKEATDKELVEHIEKILEELKKQSTDGWLEHHHHHH

SEQ ID NO: 2 - protein, 211 aa, Artificial Sequence; identical to SEQ ID NO: 1
  Synthetic peptide
  MISC_FEATURE (1)..(1): Optional residue
  MISC_FEATURE (202)..(211): Optional residues

SEQ ID NO: 3 - protein, 193 aa, Artificial Sequence
  Synthetic peptide
MDSEEVNERVKQLAEKAKEATDKEEVIEIVKELAELAKQSTDPELVLEILKQLIEVLKKSQNEELQEEILEVLKELLQLGDLEVILRAAQLAAKKGDQEVVRAALEAVAEKAIKAARKGNTDEVRKALEVALKIAEDAGTEEAVRLALEVVKRVSDEAKKQGNEDAVKEAEEVRKKIEEESGTGWLEHHHHHH

SEQ ID NO: 4 - protein, 193 aa, Artificial Sequence; identical to SEQ ID NO: 3
  Synthetic peptide
  MISC_FEATURE (1)..(1): Optional residue
  MISC_FEATURE (184)..(193): Optional residues

SEQ ID NO: 5 - protein, 200 aa, Artificial Sequence
  Synthetic peptide
MDSEEVNERVKQLAEKAKEATDKEEVIEIVKELAELAKQSTDPNLVKEIVEQLLQVAQESTDEELLETILQVIKELAKNAQSPEAALRAAEAILELAKEAGKLTEEEAKELLEIIARAAIEAARSGNVEAVRKALELALQVAKSAGTEEAVRLALEVVKRVSDEAKKQGNEDAVKEAEEVRKKIEEESGTGWLEHHHHHH

SEQ ID NO: 6 - protein, 200 aa, Artificial Sequence; identical to SEQ ID NO: 5
  Synthetic peptide
  MISC_FEATURE (1)..(1): Optional residue
  MISC_FEATURE (191)..(200): Optional residues

SEQ ID NO: 7 - protein, 220 aa, Artificial Sequence
  Synthetic peptide
MDSEEVNERVKQLAEKAKEATDKEEVIEIVKELAELAKQSTDPEVVAEIVTQLLQVAKESTDVELILEIAEVLLRLAEKAQSKELASKALSSAVEAVTYLAELLKEGPPNPEAALEAAEAALQAARLAAENGNEEAFKKAAEAALQAAKILVEVASESGDPELVEEAAKVAEEVRKLAKKQGDEEVYEKARETAREVKEELKRVREEKGDGWLEHHHHHH

SEQ ID NO: 8 - protein, 220 aa, Artificial Sequence; identical to SEQ ID NO: 7
  Synthetic peptide
  MISC_FEATURE (1)..(1): Optional residue
  MISC_FEATURE (211)..(220): Optional residues

SEQ ID NO: 9 - protein, 212 aa, Artificial Sequence
  Synthetic peptide
MDSEEVNERVKQLAEKAKEATDKEEVIEIVKELAELAKQSTDPEVISEILELLEEVARKSTDKELILEIVQVILQLAKRNHGSPLAVKAARIAAKLAADAGDAELALRAAELAVEIARTAVENGDDEVAKEAAEAALEIAKKVVEAASEKGDPELVEEAAKVAEEVRKLAKKQGDEEVYEKARETAREVKEELKRVREEKGDGWLEHHHHHH

SEQ ID NO: 10 - protein, 212 aa, Artificial Sequence; identical to SEQ ID NO: 9
  Synthetic peptide
  MISC_FEATURE (1)..(1): Optional residue
  MISC_FEATURE (203)..(212): Optional residues

SEQ ID NO: 11 - protein, 212 aa, Artificial Sequence; identical to SEQ ID NO: 9
  Synthetic peptide

SEQ ID NO: 12 - protein, 212 aa, Artificial Sequence; identical to SEQ ID NO: 9
  Synthetic peptide
  MISC_FEATURE (1)..(1): Optional residue
  MISC_FEATURE (203)..(212): Optional residues

SEQ ID NO: 13 - protein, 220 aa, Artificial Sequence
  Synthetic peptide
MDSEEVNERVKQLAEKAKEATDKEEVIEIVKELAELAKQSTDPKVVAKILQALAEVAQQSTDPELARRIIEVIAELAKESGDEALLQAAEAAKEAAQKGNTELLLAVLQALLVAVEVLIVAEQARENGNEELAEAARELIRAVAEAITEAVQQGNPELVERVARLAKKAAELIKRAIRAEKEGNRDERREALERVREVIERIEELVRQGNGWLEHHHHHH

SEQ ID NO: 14 - protein, 220 aa, Artificial Sequence; identical to SEQ ID NO: 13
  Synthetic peptide
  MISC_FEATURE (1)..(1): Optional residue
  MISC_FEATURE (211)..(220): Optional residues

SEQ ID NO: 15 - protein, 249 aa, Artificial Sequence
  Synthetic peptide
MDSEEVNERVKQLAEKAKEATDKEEVIEIVKELAELAKQSTDEKAIQEIAERLAEVAKESQDEELILTIILVLLNLLSTSTDPEALEQIARAVLELARQNGDEELAQLAEEALRAVQTAKEAKEKGDEDLAQAALLIALAAAAAAAALIAAKQTGDPEVRELAQKLVELAQTAATQVKQNPKDEEVNEALKKIVKAIQEAVESLREAEESGDPEKREKARERVREAVERAEEVQRDPSSGWLEHHHHHH

SEQ ID NO: 16 - protein, 249 aa, Artificial Sequence; identical to SEQ ID NO: 15
  Synthetic peptide
  MISC_FEATURE (1)..(1): Optional residue
  MISC_FEATURE (240)..(249): Optional residues

SEQ ID NO: 17 - protein, 250 aa, Artificial Sequence
  Synthetic peptide
MDSEEVNERVKQLAEKAKEATDKEEVIEIVKELAELAKQSTDPTLISKIAERLTEVAEQGTNDELLVQIIYVLLRILQNGQTDDLKKRVEKNAIKVLQKVVSNRDAADLAAKAVRKVAEDTLREHPDSSDVEKALKLVEEAQKAAERAREAADRTGTEDVQRLAQELIRLAIEAALQVVSDPSSEEVNEALKKIVKAIQEAVESLREAEESGDPEKREKARERVREAVERAEEVQRDPSSGWLEHHHHHH

SEQ ID NO: 18 - protein, 250 aa, Artificial Sequence; identical to SEQ ID NO: 17
  Synthetic peptide
  MISC_FEATURE (1)..(1): Optional residue
  MISC_FEATURE (241)..(250): Optional residues

SEQ ID NO: 19 - protein, 248 aa, Artificial Sequence
  Synthetic peptide
MDSEEVNERVKQLAEKAKEATDKEEVIEIVKELAELAKQSTDPEVVLEIVEQLAQVATEAQDPELVSRILEVLARLAETLTNPEALSTVIQILTELAKELLEQGNLEAAAEAIAIALEALAKTTGDEEVKRAAELAKLALQAAQEATEAAQRTGDPEVKKLAQKLAKLAATAALQILQNPDDEEVNEALKKIVKAIQEAVESLREAEESGDPEKREKARERVREAVERAEEVQRDPSSGWLEHHHHHH

SEQ ID NO: 20 - protein, 248 aa, Artificial Sequence; identical to SEQ ID NO: 19
  Synthetic peptide
  MISC_FEATURE (1)..(1): Optional residue
  MISC_FEATURE (239)..(248): Optional residues

SEQ ID NO: 21 - protein, 247 aa, Artificial Sequence
  Synthetic peptide
MDSEEVNERVKQLAEKAKEATDKEEVIEIVKELAELAKQSTDTELVKKVVSLLAEVAVESKNEELIQEIIEVLKELISSIQDPEQLKELAQELKEQLQEALEKGDYDAAKVLAEALAAAAKESGDEDLAEAAKLIAKAAEAIKRAKEAADRTGDPEVQKLAEELARLALEAALQVLQDPKDEEVNEALKKIVKAIQEAVESLREAEESGDPEKREKARERVREAVERAEEVQRDPSSGWLEHHHHHH

SEQ ID NO: 22 - protein, 247 aa, Artificial Sequence; identical to SEQ ID NO: 21
  Synthetic peptide
  MISC_FEATURE (1)..(1): Optional residue
  MISC_FEATURE (238)..(247): Optional residues

SEQ ID NO: 23 - protein, 226 aa, Artificial Sequence
  Synthetic peptide
MDSEEVNERVKQLAEKAKEATDKEEVIEIVKELAELAKQSTDEEVIKRILELLKQVLKESTDPELQARILLVLARLASQQGNLREAARLAVRAAETAAKAGDQEALKEALEIARKALEEAQQQARQAKNEGDLETLAKALIAIALAIIAAAIVACTSGDKEEAERAYEDARRVEEEARKVKESAEEQGDSEVKRLAEEAEQLAREARRHVQECRGNGWLEHHHHHH

SEQ ID NO: 24 - protein, 226 aa, Artificial Sequence; identical to SEQ ID NO: 23
  Synthetic peptide
  MISC_FEATURE (1)..(1): Optional residue
  MISC_FEATURE (217)..(226): Optional residues

SEQ ID NO: 25 - protein, 203 aa, Artificial Sequence
  Synthetic peptide
MDSEEVNERVKQLAEKAKEATDKEEVIEIVKELAELAKQSTDPTLVAKILADLAEAALEAKDPELVQRIIEILQELAKQATSEDLLTIAQLAISAARAAQNGDEAVAKVALALLQAVKLALENGNPEVAATIAKVAKKILEALKENPSDEMAKKMLELAKRVLDAAKNNDDETAREIARQAAEEVEADRENNSGWLEHHHHHH

SEQ ID NO: 26 - protein, 203 aa, Artificial Sequence; identical to SEQ ID NO: 25
  Synthetic peptide
  MISC_FEATURE (1)..(1): Optional residue
  MISC_FEATURE (194)..(203): Optional residues

SEQ ID NO: 27 - protein, 208 aa, Artificial Sequence
  Synthetic peptide
MDSEEVNERVKQLAEKAKEATDKEEVIEIVKELAELAKQSTDPEAVKEVAIQLAAVAAQAQDPELVKRIAQILEEILQQFPDDEAAREALQIARAILIVLEALHSSNSEEFKKVAKALLEAVLLALENGDPKVALEIARAAEAIIRALRENPSDEMAKKMLELAKRVLDAAKNNDDETAREIARQAAEEVEADRENNSGWLEHHHHHH

SEQ ID NO: 28 - protein, 208 aa, Artificial Sequence; identical to SEQ ID NO: 27
  Synthetic peptide
  MISC_FEATURE (1)..(1): Optional residue
  MISC_FEATURE (199)..(208): Optional residues

SEQ ID NO: 29 - protein, 229 aa, Artificial Sequence
  Synthetic peptide
MDIEKLCKKAESEAREARSKAEELRQRHPDSQAARDAQKLASQAEEAVKLACELAQEHPNADRAKACILLASAAAYAASKAVEDAQRHPDNQTARDKIKEASRIAELVIQFCRAAQENNDQKALDVLEKLATVASESGNEHVLKIIVEVLAILAQTITNKDDVIQAVDIARKIAEESTNSELVNEIVKQLEEVAKEATDKELVEHIEKILEELKKQSTDGWLEHHHHHH

SEQ ID NO: 30 - protein, 229 aa, Artificial Sequence; identical to SEQ ID NO: 29
  Synthetic peptide
  MISC_FEATURE (1)..(1): Optional residue
  MISC_FEATURE (220)..(229): Optional residues

SEQ ID NO: 31 - protein, 191 aa, Artificial Sequence
  Synthetic peptide
MDSEEEQERIRRILKEARKSGTEESLRQAIEDVAQLAKKSQDPEVIAHAVHVIAKIAQTSGSEEAKQQALRAVTEILSNASEEEILEALKEALETAQQEGDDEALKLLVAAAAAAAKNSKDPDAIKEIVQLLLEAAKNSTDSELVNEIVKQLEEVAKEATDKELVEHIEKILEELKKQSTDGWLEHHHHHH

SEQ ID NO: 32 - protein, 191 aa, Artificial Sequence; identical to SEQ ID NO: 31
  Synthetic peptide
  MISC_FEATURE (1)..(1): Optional residue
  MISC_FEATURE (182)..(191): Optional residues

SEQ ID NO: 33 - protein, 233 aa, Artificial Sequence
  Synthetic peptide
MDSEEEQERIRRILKEARKSGTEESLRQAIEDVAQLAKKSQDPEVLRTAVEVIKEIAETSGSPEALYEAIQAVIEIARSAQDEEALATAAIAAAELADQLLQTASESGDEEALTEAAELAREILREARRVLEQAQRSGNLEVAAKALIAIALAILVIAKVACQKGDKEEAERAYEDARRVEEEARKVKESAEEQGDSEVKRLAEEAEQLAREARRHVQECRGNGWLEHHHHHH

SEQ ID NO: 34 - protein, 233 aa, Artificial Sequence; identical to SEQ ID NO: 33
  Synthetic peptide
  MISC_FEATURE (1)..(1): Optional residue
  MISC_FEATURE (224)..(233): Optional residues

SEQ ID NO: 35 - protein, 260 aa, Artificial Sequence
  Synthetic peptide
MSNDEKEKLKELLKRAEELAKSPDPEDLKEAVRLAEEVVRERPGSEDAKKALKIVIKAAAELAKAPNPEALKEAIEALQKVAEHSNSEEVKEAIEAIKSVLEAAREALESGDEEAAQELARLAYRAAQLLIKLEDSQDDEEKKALLLAVQALAAAAQALQAASQTGDPEVIELAQKLVELAETAATQVEQNPKDEEVNEALKKIVKAIQEAVESLREAEESGDPEKREKARERVREAVERAEEVQRDPSSGWLEHHHHHH

SEQ ID NO: 36 - protein, 260 aa, Artificial Sequence; identical to SEQ ID NO: 35
  Synthetic peptide
  MISC_FEATURE (1)..(1): Optional residue
  MISC_FEATURE (251)..(260): Optional residues

SEQ ID NO: 37 - protein, 234 aa, Artificial Sequence
  Synthetic peptide
MTTEDERRELEKVARKAIEAAREGNTDEVREQLQRALEIARESGTKTAVKLALDVALRVAQEAAKRGNKDAIDEAAEVVVRIAEESNNSDALEQALRVLEEIAKAVLKSEKTEDAKKAVKLVQEAYKAAQRAIEAAKRTGTPDVIKLAIKLAKLAARAALEVIKRPKSEEVNEALKKIVKAIQEAVESLREAEESGDPEKREKARERVREAVERAEEVQRDPSSGWLEHHHHHH

SEQ ID NO: 38 - protein, 234 aa, Artificial Sequence; identical to SEQ ID NO: 37
  Synthetic peptide
  MISC_FEATURE (1)..(1): Optional residue
  MISC_FEATURE (225)..(234): Optional residues

SEQ ID NO: 39 - protein, 218 aa, Artificial Sequence
  Synthetic peptide
MSSDEEEARELIERAKEAAERAQEAAERTGDPRVRELARELKRLAQEAAEEVKRDPSSRITLDILKAVIEAIEVAVRSLEKAYRNGNPEDVKKASKIVEEAVRLAEAATKGNYQEINKAAREATKNNNEDLVRIAVKAAAAAAKETQTKDDVKKIVDELRKIAKNNTNSELVNEIVKQLEEVAKEATDKELVEHIEKILEELKKQSTDGWLEHHHHHH

SEQ ID NO: 40 - protein, 218 aa, Artificial Sequence; identical to SEQ ID NO: 39
  Synthetic peptide
  MISC_FEATURE (1)..(1): Optional residue
  MISC_FEATURE (209)..(218): Optional residues

SEQ ID NO: 41 - protein, 222 aa, Artificial Sequence
  Synthetic peptide
MSSDEEEARELIERAKEAAERAQEAAERTGDPRVRELARELKRLAQEAAEEVKRDPSSKTTLIALKLIIIAIELAVRALEEAIKKGNPEEVKKATKIVEKAVRLAEEIQHGNQKQIARAAADIAKLAIESGNEDVARKVVKVVAELAQTGTNKDVVTEIVKALEKIARQGTNSELVNEIVKQLEEVAKEATDKELVEHIEKILEELKKQSTDGWLEHHHHHH

SEQ ID NO: 42 - protein, 222 aa, Artificial Sequence; identical to SEQ ID NO: 41
  Synthetic peptide
  MISC_FEATURE (1)..(1): Optional residue
  MISC_FEATURE (213)..(222): Optional residues

SEQ ID NO: 43 - protein, 234 aa, Artificial Sequence
  Synthetic peptide
MSSDEEEARELIERAKEAAERAQEAAERTGDPRVRELARELKRLAQEAAEEVKRDPSSKDTLRALSIIIIAIEVAVIALEVAQKQGNPKVKERASQLVEEAVRAAEEVQNDPTDDAVYNAVHTLARAALDAVKNGPDTRDVVKKALEVVARLAIIAARQGSTDAVRDALKVALKIARTAGNEEAVRLALEVVKRVSDEAKKQGNEDAVKEAEEVRKKIEEESGTGWLEHHHHHH

SEQ ID NO: 44 - protein, 234 aa, Artificial Sequence; identical to SEQ ID NO: 43
  Synthetic peptide
  MISC_FEATURE (1)..(1): Optional residue
  MISC_FEATURE (225)..(234): Optional residues

SEQ ID NO: 45 - protein, 236 aa, Artificial Sequence
  Synthetic peptide
MDSEEVNERVKQLAEKAKEATDKEEVIEIVKELAELAKQSTDPNLVAEVVRALTEVAKTSTDTELIREIIKVLLELASKLRDPQAVLEALQAVAELARELAEKTGDPIAKECAEAVSAAAEAVKKAADLLKRHPGSEAAQAALELAKAAAEAVLIACLLALDYPKSDIAKKCIKAASEAAEEASKAAEEAQRHPDSQKARDEIKEASQKAEEVKERCERAQEHPNAGWLEHHHHHH

SEQ ID NO: 46 - protein, 236 aa, Artificial Sequence; identical to SEQ ID NO: 45
  Synthetic peptide
  MISC_FEATURE (1)..(1): Optional residue
  MISC_FEATURE (227)..(236): Optional residues

SEQ ID NO: 47 - protein, 242 aa, Artificial Sequence
  Synthetic peptide
MDSEEVNERVKQLAEKAKEATDKEEVIEIVKELAELAKQSTDPNVVAEIVYQLAEVAEHSTDPELIKEILQEALRLAEEQGDEELAEAARLALKAARLLEEARQLLSKDPENEAAKECLKAVRAALEAALLALLLLAKHPGSQAAQDAVQLATAALRAVEAACQLAKQYPNSDIAKKCIKAASEAAEEASKAAEEAQRHPDSQKARDEIKEASQKAEEVKERCERAQEHPNAGWLEHHHHHH

SEQ ID NO: 48 - protein, 242 aa, Artificial Sequence; identical to SEQ ID NO: 47
  Synthetic peptide
  MISC_FEATURE (1)..(1): Optional residue
  MISC_FEATURE (233)..(242): Optional residues

SEQ ID NO: 49 - protein, 199 aa, Artificial Sequence
  Synthetic peptide
MDSEEVNERVKQLAEKAKEATDKEEVIEIVKELAELAKQSTDKEVVKRIVELLTEVAKESTDVELIAEIIAVLIELAAHASSETLQEANQLIRELLHEAASGNKEAVQILLEAIAELAVKAARKGNVEAVKLALQAALEVAESAGTEEAVRLALEVVKRVSDEAKKQGNEDAVKEAEEVRKKIEEESGTGWLEHHHHHH

SEQ ID NO: 50 - protein, 199 aa, Artificial Sequence; identical to SEQ ID NO: 49
  Synthetic peptide
  MISC_FEATURE (1)..(1): Optional residue
  MISC_FEATURE (190)..(199): Optional residues

SEQ ID NO: 51 - protein, 215 aa, Artificial Sequence
  Synthetic peptide
MDSEEVNERVKQLAEKAKEATDKEEVIEIVKELAELAKQSTDEELVNRIVEALEEVAKESTDPQLIIEILLVLALLAVESGGTEKADEALRRITEQAREAAQQGDAEAVLEAARAALQAAKAAAEKGDDEVFKSAAEAALTIAKELVEAASEKGDPELVEEAAKVAEEVRKLAKKQGDEEVYEKARETAREVKEELKRVREEKGDGWLEHHHHHH

SEQ ID NO: 52 - protein, 215 aa, Artificial Sequence; identical to SEQ ID NO: 51
  Synthetic peptide
  MISC_FEATURE (1)..(1): Optional residue
  MISC_FEATURE (206)..(215): Optional residues

SEQ ID NO: 53 - protein, 232 aa, Artificial Sequence
  Synthetic peptide
MDSEEVNERVKQLAEKAKEATDKEEVIEIVKELAELAKQSTDPEVVKEIVEQLLQVAQEAQDPELVKEIIRILKELAKTAENEEAAATALLAVAEALAVLAELLARTTGDDSARQAAELAKEAAEAAKRAQEAAKRTGDPEVKRLALELVRLAAEAAEEVTKNPDDEEVNEALKKIVKAIQEAVESLREAEESGDPEKREKARERVREAVERAEEVQRDPSSGWLEHHHHHH

SEQ ID NO: 54 - protein, 232 aa, Artificial Sequence; identical to SEQ ID NO: 53
  Synthetic peptide
  MISC_FEATURE (1)..(1): Optional residue
  MISC_FEATURE (223)..(232): Optional residues

SEQ ID NO: 55 - protein, 234 aa, Artificial Sequence
  Synthetic peptide
MDSEEVNERVKQLAEKAKEATDKEEVIEIVKELAELAKQSTDPTLVAKIAVLLAEVAAEAQDPELIKRILEILRQLIKNAKSDEARKAAKALAEAVEVALKAAQQLKQNPEDESARQALELILEAVEAAARALKAALETGSPEVIELALKLAELAIEAARQVLKNPDNEEVNEALKKIVKAIQEAVESLREAEESGDPEKREKARERVREAVERAEEVQRDPSSGWLEHHHHHH

SEQ ID NO: 56 - protein, 234 aa, Artificial Sequence; identical to SEQ ID NO: 55
  Synthetic peptide
  MISC_FEATURE (1)..(1): Optional residue
  MISC_FEATURE (225)..(234): Optional residues

SEQ ID NO: 57 - protein, 202 aa, Artificial Sequence
  Synthetic peptide
MDSEEVNERVKQLAEKAKEATDKEEVIEIVKELAELAKQSTDKEAIKDIVRALKEVLKHSQDDELREQILIVLALLAAQAGDVEEALEALERLAQEAKEKGDEEALKVLKALAEAVRTAKENGNPEVAATVAEAAAKIATALRENPSDEMAKKMLELAKRVLDAAKNNDDETAREIARQAAEEVEADRENNSGWLEHHHHHH

SEQ ID NO: 58 - protein, 202 aa, Artificial Sequence; identical to SEQ ID NO: 57
  Synthetic peptide
  MISC_FEATURE (1)..(1): Optional residue
  MISC_FEATURE (193)..(202): Optional residues

SEQ ID NO: 59 - protein, 204 aa, Artificial Sequence
  Synthetic peptide
MDSEEVNERVKQLAEKAKEATDKEEVIEIVKELAELAKQSTDEEAVKEVVRQLALVAATATDPELIAEILQVILQLAEQAGDEEVAEAARQALEEIKQAQEQGSEAVALVLAALAVAVLAAAANGNPEVARVVKHAARLIKEALEENPSDEMAKKMLELAKRVLDAAKNNDDETAREIARQAAEEVEADRENNSGWLEHHHHHH

SEQ ID NO: 60 - protein, 204 aa, Artificial Sequence; identical to SEQ ID NO: 59
  Synthetic peptide
  MISC_FEATURE (1)..(1): Optional residue
  MISC_FEATURE (195)..(204): Optional residues

SEQ ID NO: 61 - protein, 201 aa, Artificial Sequence
  Synthetic peptide
MDSEEVNERVKQLAEKAKEATDKEEVIEIVKELAELAKQSTDKKLALQIVLLLAEVLQEAQDPELAIRIAEELAEIIKEAGGSEDALQIVQEIATALRQGNEEVAKVLAVLLIAVILALQNGNPEVAHEVARVAREILKALEENPTDEMAKKMLELAKRVLDAAKNNDDETAREIARQAAEEVEADRENNSGWLEHHHHHH

SEQ ID NO: 62 - protein, 201 aa, Artificial Sequence; identical to SEQ ID NO: 61
  Synthetic peptide
  MISC_FEATURE (1)..(1): Optional residue
  MISC_FEATURE (192)..(201): Optional residues

SEQ ID NO: 63 - protein, 222 aa, Artificial Sequence
  Synthetic peptide
MDSEEEQERIRRILKEARKSGTEESLRQAIEDVAQLAKKSQDEEVLREAVEVITQAARDSGSEEALQQAVRAVLEIAKSGKDVEAAAHAAKLLLEKNPEDESAREALELVERAVQAAQEAQEAANRTGDPEVQELAEKLLALAADAAAQVVKNPDDEEVNEALKKIVKAIQEAVESLREAEESGDPEKREKARERVREAVERAEEVQRDPSSGWLEHHHHHH

SEQ ID NO: 64 - protein, 222 aa, Artificial Sequence; identical to SEQ ID NO: 63
  Synthetic peptide
  MISC_FEATURE (1)..(1): Optional residue
  MISC_FEATURE (213)..(222): Optional residues

SEQ ID NO: 65 - protein, 247 aa, Artificial Sequence
  Synthetic peptide
MSYEDECEEKARRVAEKVERLKRSGTSEDEIAEEVAREISEVIRTLKESGSSDEEIATCVALILAAAARALKESGVSDEQINRILATLIKEVLRALNQETNKSNEEILRELLQALIELASKSDSETALLAVQLVVVLAKVALEVAQSEGSEEALELALEAAEEAARLAKEVLRLATENGNPEVARRAVELVKRVAELLERIARESGSEEAKERAERVREEARELQERVKELREREGDGWLEHHHHHH

SEQ ID NO: 66 - protein, 247 aa, Artificial Sequence; identical to SEQ ID NO: 65
  Synthetic peptide
  MISC_FEATURE (1)..(1): Optional residue
  MISC_FEATURE (236)..(247): Optional residues

SEQ ID NO: 67 - protein, 220 aa, Artificial Sequence
  Synthetic peptide
MSNDEKEKLKELLKRAEELAKSPDPEDLKEAVRLAEEVVRERPGSEAAKKALEIIQEAAELLKKSPDPEAIIAAARALLKIAATTGDNEAAKQAIEAASKAAQLAEQRGDDELVCEALALLIAAQVLLLKQQGTSDEEVAEHVARTISQLVQRLKRKGASYEVIKECVQRIVEEIVEALKRSGTSEDEINEIVRRVKSEVERTLKESGSSGWLEHHHHHH

SEQ ID NO: 68 - protein, 220 aa, Artificial Sequence; identical to SEQ ID NO: 67
  Synthetic peptide
  MISC_FEATURE (1)..(1): Optional residue
  MISC_FEATURE (211)..(220): Optional residues

SEQ ID NO: 69 - protein, 371 aa, Artificial Sequence
  Synthetic peptide
SVLGKVLIMAALVGNKDVVKVLIEVGADVNASLVSGATPLHAAAMNGHKEVVKLLISKGADVNALDEVGWTPLHLAVWVVLEIVECLLKNGADVNAADIDGYTPLHLAAFSGHLEIVEVLLKYGADVNADDQAGFTPLHLAAIFGHKEVVKLLISKGADLNTSAKDGATPVLLALRRGDEEVVRLLKEEAKKRGDEFLARCAEAAELAIEALKLAEELLRRYPNDEAARLAHHLAKLALEAVELACILASEHPNADIAKLCIKAASEAAEAASKAAELAQRHPDSQAARDAIKLASQAAEAVKLACELAQEHPNADIAKKCIKAASEAAEEASKAAEEAQRHPDSQKARDEIKEASQKAEEVKERCERAQE

SEQ ID NO: 70 - protein, 353 aa, Artificial Sequence
  Synthetic peptide
SVLGKVLIMAALVGNKDVVKVLIEVGADVNASLVSGATPLHAAAMNGHKEVVKLLISKGADVNALDEVGWTPLHLAVWVVLEIVECLLKNGADVNAADIDGYTPLHLAAFSGHLEIVEVLLKYGADVNADDQAGFTPLHLAAIFGHKEVVKLLISKGADLNTSAKDGATPAALAASSGDKDVVETLERQARRNGDKELAQLAEVAREIYRLAEEARKLAKDEEEAKKIQKAANEAIAALALAVEKVTDNEVIEKLLEVVKEIIRLAEEAMKKMTDEEEAAKIAKEALEAIKMLARAVEEVTDKERIEQLLREVKEEIRRAEEESRKETDDEEAAKRAREALRRIRERAREVEE

SEQ ID NO: 71 - protein, 324 aa, Artificial Sequence
  Synthetic peptide
SVLGKVLIMAALVGNKDVVKVLIEVGADVNASLVSGATPLHAAAMNGHKEVVKLLISKGADVNALDEVGWTPLHLAVWVVLEIVECLLKNGADVNAADIDGYTPLHLAAFSGHLEIVEVLLKYGADVNADDQAGFTPLHLAAIFGHKEVVKLLISKGADLNTSAKDGATPLLLAVRRGDEEAIRELLRELIERARESEEQAKRILHIILLAAEEAARRGNEEILRLALEAALEVARRSGTTEAVKLALEVVARVAIEAARRGNTDAVREALEVALEIARESGTEEAVRLALEVVKRVSDEAKKQGNEDAVKEAEEVRKKIEEES

SEQ ID NO: 72 - protein, 339 aa, Artificial Sequence
  Synthetic peptide
SVLGKVLIMAALVGNKDVVKVLIEVGADVNASLVSGATPLHAAAMNGHKEVVKLLISKGADVNALDEVGWTPLHLAVWVVLEIVECLLKNGADVNAADIDGYTPLHLAAFSGHLEIVEVLLKYGADVNADDQAGFTPLHLAAIFGHKEVVKLLISKGADLNTSAKDGATPIALAIKRGDEEVAEKLIRSSSEEIIIEAARLAIEIARELLKKGDEELALRAARIALRAVRRLEEEARRTGSTEVLIEAARLAIEVARVALKVGSPETAREAVRTALELVQELERQARKTGSDEVLKRAAELAKEVARVAKEVGSPETARQARETAERLREELRRNREKK

SEQ ID NO: 73 - protein, 345 aa, Artificial Sequence
  Synthetic peptide
SVLGKVLIMAALVGNKDVVKVLIEVGADVNASLVSGATPLHAAAMNGHKEVVKLLISKGADVNALDEVGWTPLHLAVWVVLEIVECLLKNGADVNAADIDGYTPLHLAAFSGHLEIVEVLLKYGADVNADDQAGFTPLHLAAIFGHKEVVKLLISKGADLNTSAKDGATPLLFAIKRGDEEAVRILLEELERRGEHNKEEALIAARIALKVAEIARRQGNEELFKEAAEIALRLAKLLVRIAKKEGDPELVLEAAKVALRVAELAAKNGDKEVFKKAAESALEVAKRLVEVASKEGDPELVEEAAKVAEEVRKLAKKQGDEEVYEKARETAREVKEELKRVREEK

SEQ ID NO: 74 - protein, 326 aa, Artificial Sequence
  Synthetic peptide
SVLGKVLIMAALVGNKDVVKVLIEVGADVNASLVSGATPLHAAAMNGHKEVVKLLISKGADVNALDEVGWTPLHLAVWVVLEIVECLLKNGADVNAADIDGYTPLHLAAFSGHLEIVEVLLKYGADVNADDQAGFTPLHLAAIFGHKEVVKLLISKGADLNTSAKDGATPVILAARRGDEEVIELLLREAEKRGDEELLVIARLAQAIAIAKKNGNEEVAKEILKAALIIYEALRENNSDEMAKVMLALAKAVLLAAKNNDDEVAREIARAAAEIVEALRENNSDEMAKKMLELAKRVLDAAKNNDDETAREIARQAAEEVEADRE

SEQ ID NO: 75 - protein, 300 aa, Artificial Sequence
  Synthetic peptide
SDIEEIRQLAEELRKKSDNEEVRKLAQEAAELAKRSTDSDVLEIVKDALELAKQSTNEEVIKLALKAAVLAAKSTDEEILKIVLEALRKARKSTNEEEILLILRAAVLAAKGDLEEALIIAARRGDEELVELARRGGADVNASLVSGATPLHAAAMNGHKEVVKLLISKGADVNALDEVGWTPLHLAVWVVLEIVECLLKNGADVNAADIDGYTPLHLAAFSGHLEIVEVLLKYGADVNADDQAGFTPLHLAAIFGHKEVVKLLISKGADLNTSAKDGATPLDMARESGNEEVVKLLEKQ

SEQ ID NO: 76 - protein, 303 aa, Artificial Sequence
  Synthetic peptide
SEKEKVEELAQRIREQLPDTELAREAQELADEARKSDDSEALKVVYLALRIVQQLPDTELAREALELAKEAVKSTDEEILKAIYHALELVRRFPNTELAEAALLAALARQRGDEELAEKALILAAKRGSEEVVELARRAGADVNASLVSGATPLHAAAMNGHKEVVKLLISKGADVNALDEVGWTPLHLAVWVVLEIVECLLKNGADVNAADIDGYTPLHLAAFSGHLEIVEVLLKYGADVNADDQAGFTPLHLAAIFGHKEVVKLLISKGADLNTSAKDGATPLDMARESGNEEVVKLLEKQ

SEQ ID NO: 77 - protein, 324 aa, Artificial Sequence
  Synthetic peptide
SVAEEIEKRAKKISKELKKEGKNPEWIEELQRAADKLVEVARRATSSDALEIAKRAVKIAEELAKQGSNPKWIAELLKAAAKLVEVAARATSEEALEIAKLAIKIAEELAKRGHDPEEIAEILKEAAKAVELARRGNLEEALIIAAKRGNEEIVEEARRGGADVNASLVSGATPLHAAAMNGHKEVVKLLISKGADVNALDEVGWTPLHLAVWVVLEIVECLLKNGADVNAADIDGYTPLHLAAFSGHLEIVEVLLKYGADVNADDQAGFTPLHLAAIFGHKEVVKLLISKGADLNTSAKDGATPLDMARESGNEEVVKLLEKQ

SEQ ID NO: 78 - protein, 549 aa, Artificial Sequence
  Synthetic peptide
SELGKRLIEAAENGNKDRVKDLIENGADVNASDSDGRTPLHHAAENGHKEVVKLLISKGADVNAKDSDGRTPLHHAAENGHKEVVKLLISKGADVNAKDSDGRTPLHHAAENGHKEVVKLLISKGADVNAKADRGMTPLHFAAWRGHKEVVKLLISKGADLNTSAKDGATPVLLALRRGDEEVVRLLKEEAKKRGDEFLARCAEAAELAIEALKLAEELLRRYPNDEAARLAHHLAKLALEAVELACILASEHPNADIAKLCIKAASEAAEAASKAAELAQRHPDSQAARDAIKLASQAAEAVKLACELAQEHPNADIAKLCIIAASLAAEAASKAAELAQRHPDSQAARDAIKLASQAAEAVKLACELAQEHPNAIIAILCIVAAIAAAIAASMAAALAQRHPDSQAARDAIKLASQAAEAVKLACELAQEHPNAKIAVLCILAAALAAIAAALAALLAQLHPDSQAARDAIKLASQAAEAVKLACELAQEHPNADIAEKCILLAILAALLAILAALLAMLHPDSQLARDLIDLASELAEEVKERCER

SEQ ID NO: 79 - protein, 173 aa, Artificial Sequence
  Synthetic peptide
  MISC_FEATURE (1)..(1): Optional residue
MSSEDTVRKIAQKCSEAIRESNDCEEAARKCAKTISEAIRESNSSELAVRIIAQVCSEAIRESNDCECAARICAKIISEAIRESNSSELAVRIIAQVCSEAIRESNDCECAARICAKIISEAIRESNSSELAKRIIKQVCSEAKRESNDTECAKRICTKIKSEAKRESNSWLE

SEQ ID NO: 80 - protein, 202 aa, Artificial Sequence
  Synthetic peptide
  MISC_FEATURE (1)..(1): Optional residue
MSYEDECEEKARRVAEKVERLKRSGTSEDEIAEEVAREISEVIRTLKESGSSYEVICECVARIVAEIVEALKRSGTSEDEIAEIVARVISEVIRTLKESGSSYEVICECVARIVAEIVEALKRSGTSEDEIAEIVARVISEVIRTLKESGSSYEVIKECVQRIVEEIVEALKRSGTSEDEINEIVRRVKSEVERTLKESGSS

SEQ ID NO: 81 - protein, 201 aa, Artificial Sequence
  Synthetic peptide
  MISC_FEATURE (1)..(1): Optional residue
MSSEKEELRERLVKICVENAKRKGDDTEEAREAAREAFELVREAAERAGIDSSEVLELAIRLIKECVENAQREGYDISEACRAAAEAFKRVAEAAKRAGITSSEVLELAIRLIKECVENAQREGYDISEACRAAAEAFKRVAEAAKRAGITSSETLKRAIEEIRKRVEEAQREGNDISEACRQAAEEFRKKAEELKRRGDG

SEQ ID NO: 82 - protein, 170 aa, Artificial Sequence
  Synthetic peptide
  MISC_FEATURE (1)..(1): Optional residue
MSTKEDARSTCEKAARKAAESNDEEVAKQAAKDCLEVAKQAGMPTKEAARSFCEAAARAAAESNDEEVAKIAAKACLEVAKQAGMPTKEAARSFCEAAARAAAESNDEEVAKIAAKACLEVAKQAGMPTKEAARSFCEAAKRAAKESNDEEVEKIAKKACKEVAKQAGMP

SEQ ID NO: 83 - protein, 182 aa, Artificial Sequence
  Synthetic peptide
  MISC_FEATURE (1)..(1): Optional residue
MSDEMKKVMEALKKAVELAKKNNDDEVAREIERAAKEIVEALRENNSDEMAKVMLALAKAVLLAAKNNDDEVAREIARAAAEIVEALRENNSDEMAKVMLALAKAVLLAAKNNDDEVAREIARAAAEIVEALRENNSDEMAKKMLELAKRVLDAAKNNDDETAREIARQAAEEVEADRENNS

SEQ ID NO: 84 - protein, 202 aa, Artificial Sequence
  Synthetic peptide
  MISC_FEATURE (1)..(1): Optional residue
MSYEDEAEEKARRVAEKVERLKRSGTSEDEIAEEVAREISEVIRTLKESGSSYEVIAEIVARIVAEIVEALKRSGTSEDEIAEIVARVISEVIRTLKESGSSYEVIAEIVARIVAEIVEALKRSGTSEDEIAEIVARVISEVIRTLKESGSSYEVIKEIVQRIVEEIVEALKRSGTSEDEINEIVRRVKSEVERTLKESGSS

SEQ ID NO: 85 - protein, 201 aa, Artificial Sequence
  Synthetic peptide
  MISC_FEATURE (1)..(1): Optional residue
MSSEKEELRERLVKIVVENAKRKGDDTEEAREAAREAFELVREAAERAGIDSSEVLELAIRLIKEVVENAQREGYDISEAARAAAEAFKRVAEAAKRAGITSSEVLELAIRLIKEVVENAQREGYDISEAARAAAEAFKRVAEAAKRAGITSSETLKRAIEEIRKRVEEAQREGNDISEAARQAAEEFRKKAEELKRRGDG

SEQ ID NO: 86 - protein, 163 aa, Artificial Sequence
  Synthetic peptide
  MISC_FEATURE (1)..(1): Optional residue
MDSEEVNERVKQLAEKAKEATDKEEVIEIVKELAELAKQSTDSELVNEIVKQLAEVAKEATDKELVIYIVKILAELAKQSTDSELVNEIVKQLAEVAKEATDKELVIYIVKILAELAKQSTDSELVNEIVKQLEEVAKEATDKELVEHIEKILEELKKQSTDG

SEQ ID NO: 87 - protein, 187 aa, Artificial Sequence
  Synthetic peptide
  MISC_FEATURE (1)..(1): Optional residue
MNDERQKQREEVRKLAEELASKATDEELIKEIKKCAQLAEELASRSTNDELIKQILEVAKLAFELASKATDEELIKEILKCCQLAFELASRSTNDELIKQILEVAKLAFELASKATDEELIKEILKCCQLAFELASRSTNDEEIKQILETAKEAFERASKATDEEEIKEILKKCQEKFEKKSRSTNG

SEQ ID NO: 88 - protein, 241 aa, Artificial Sequence
  Synthetic peptide
  MISC_FEATURE (1)..(1): Optional residue
MDIEKLCKKAESEAREARSKAEELRQRHPDSQAARDAQKLASQAEEAVKLACELAQEHPNADIAKLCIKAASEAAEAASKAAELAQRHPDSQAARDAIKLASQAAEAVKLACELAQEHPNADIAKLCIKAASEAAEAASKAAELAQRHPDSQAARDAIKLASQAAEAVKLACELAQEHPNADIAKKCIKAASEAAEEASKAAEEAQRHPDSQKARDEIKEASQKAEEVKERCERAQEHPNA

SEQ ID NO: 89 - protein, 154 aa, Artificial Sequence
  Synthetic peptide
  MISC_FEATURE (1)..(1): Optional residue
MSDIEEIRQLAEELRKKSDNEEVRKLAQEAAELAKRSTDSDVLEIVKDALELAKQSTNEEVIKLALKAAVLAAKSTDSDVLEIVKDALELAKQSTNEEVIKLALKAAVLAAKSTDEEVLEEVKEALRRAKESTDEEEIKEELRKAVEEAESTDG

SEQ ID NO: 90 - protein, 154 aa, Artificial Sequence
  Synthetic peptide
  MISC_FEATURE (1)..(1): Optional residue
MSEKEKVEELAQRIREQLPDTELAREAQELADEARKSDDSEALKVVYLALRIVQQLPDTELAREALELAKEAVKSTDSEALKVVYLALRIVQQLPDTELAREALELAKEAVKSTDQEALKSVYEALQRVQDKPNTEEARESLERAKEDVKSTDG

SEQ ID NO: 91 - protein, 162 aa, Artificial Sequence
  Synthetic peptide
  MISC_FEATURE (1)..(1): Optional residue
MSDSEKLAKRVLKELKRRGTSDEELERMKRELEKIIKSATSSDAMRLALRVVLELVRRGTSSEILEKMMRMLIKIIQSATSSDAMRLALRVVLELVRRGTSSEILEKMMRMLIKIIQSATSDDQMREALRQVLEEVRKGTSSEQLERSMRKLIKEIKKRTSG

SEQ ID NO: 92 - protein, 186 aa, Artificial Sequence
  Synthetic peptide
  MISC_FEATURE (1)..(1): Optional residue
MSEAEELARRAAKEAKELCKRSTDEELCKELKKLAELLKELAERYPDSEAAKLALKAALEAIELCKQSTDEELCEELVKLAQKLIELAKRYPDSEAAKLALKAALEAIELCKQSTDEELCEELVKLAQKLIELAKRYPDSEEAKRALKEAKELIEQCKESTDEDECRELVKRAEELIREAKENPDG

SEQ ID NO: 93 - protein, 210 aa, Artificial Sequence
  Synthetic peptide
  MISC_FEATURE (1)..(1): Optional residue
MDECERLRQEVEKAEKELEKLAKQSTDEEVRQIAREVAKQLRRLAEEACRSNSDECLRLASEVVKAVQELVKLAEQATDEEVIRVALEVARELIRLAQEACRSNDDECLRLASEVVKAVQELVKLAEQATDEEVIRVALEVARELIRLAQEACRSNDEECLREASEVVKEVQELVKEAEKSTDEEEIRELLQRAEERIREAQERCREGDG

SEQ ID NO: 94 - protein, 218 aa, Artificial Sequence
  Synthetic peptide
  MISC_FEATURE (1)..(1): Optional residue
MTRQKEQLDEVLEEIQRLAEEARKLMTDEEEAKKIQEEAERAKEMLRRAVEKVTDNEVIEKLLEVVKEIIRLAEEAMKKMTDEEEAAKIAKEALEAIKMLARAVEEVTDNEVIEKLLEVVKEIIRLAEEAMKKMTDEEEAAKIAKEALEAIKMLARAVEEVTDKERIEQLLREVKEEIRRAEEESRKETDDEEAAKRAREALRRIRERAREVEEDKSG

SEQ ID NO: 95 - protein, 166 aa, Artificial Sequence
  Synthetic peptide
  MISC_FEATURE (1)..(1): Optional residue
MDSYTERARKAVKRYVKEEGGSEEEAEREAEKVREEIRKKASDSYLIQAAAAVVAYVIEEGGSPEEAVKIAEEVVRRIKEKADDSYLIQAAAAVVAYVIEEGGSPEEAVKIAEEVVRRIKEKADDRELIRRAAERVAEVIERGGSPEEAVKEAEKEVKKQKEESDG

SEQ ID NO: 96 - protein, 166 aa, Artificial Sequence
  Synthetic peptide
  MISC_FEATURE (1)..(1): Optional residue
MSIQEKAKQSVIRKVKEEGGSEEEARERAKEVEERLKKEADDSTLVRAAAAVVLYVLEKGGSTEEAVQRAREVIERLKKEASDSTLVRAAAAVVLYVLEKGGSTEEAVQRAREVIERLKKEASDEELIREAAKEVLKVLEEGGSVEEAVERARERIEELQKRSDDG

SEQ ID NO: 97 - protein, 166 aa, Artificial Sequence
  Synthetic peptide
  MISC_FEATURE (1)..(1): Optional residue
MSDLEKALKRFVKEEKKKGRNPEEAKKEAKKLKKKLKKSAGSSDLLTALAKFVLEEVRKGRNPEEAVKEAIKLAEKLKRSAGSSDLLTALAKFVLEEVRKGRNPEEAVKEAIKLAEKLKRSAGSSEQLEKLATKVLEEVKKGRNPKRAVEEAIKQAKEDRKRSNSG

SEQ ID NO: 98 - protein, 170 aa, Artificial Sequence
  Synthetic peptide
  MISC_FEATURE (1)..(1): Optional residue
MSDLQEVADRIVEQLKREGRSPEEARKEARRLIEEIKQSAGGDSELIEVAVRIVKELEEQGRSPSEAAKEAVELIERIRRAAGGDSELIEVAVRIVKELEEQGRSPSEAAKEAVELIERIRRAAGGDSDRIKKAVELVRELEERGRSPSEAARRAVEEIQRSVEEDGGNG

SEQ ID NO: 99 - protein, 158 aa, Artificial Sequence
  Synthetic peptide
  MISC_FEATURE (1)..(1): Optional residue
MSTKEEKERIERIEKEVRSPDPENIREAVRKAEELLRENPSTEAEELLRRAIEAAVRAPDPEAIREAVRAAEELLRENPSTEAEELLRRAIEAAVRAPDPEAIREAVRAAEELLRENPSEEAKELLRRAIESAKKAPDPEAQREAKRAEEELRKEDPG

SEQ ID NO: 100 - protein, 158 aa, Artificial Sequence
  Synthetic peptide
  MISC_FEATURE (1)..(1): Optional residue
MSTKEEKERIERIEKEVRSPDCENIREAVRKAEELLRENPSTEAEELLRRAIEAAVRCPDCEAIREAVRAAEELLRENPSTEAEELLRRAIEAAVRCPDCEAIREAVRAAEELLRENPSEEAKELLRRAIESAKKCPDPEAQREAKRAEEELRKEDPG

SEQ ID NO: 101 - protein, 162 aa, Artificial Sequence
  Synthetic peptide
  MISC_FEATURE (1)..(1): Optional residue
MDSEEEQERIRRILKEARKSGTEESLRQAIEDVAQLAKKSQDSEVLEEAIRVILRIAKESGSEEALRQAIRAVAEIAKEAQDSEVLEEAIRVILRIAKESGSEEALRQAIRAVAEIAKEAQDPRVLEEAIRVIRQIAEESGSEEARRQAERAEEEIRRRAQG

SEQ ID NO: 102 - protein, 174 aa, Artificial Sequence
  Synthetic peptide
  MISC_FEATURE (1)..(1): Optional residue
MQCEDRKEKIRELERKARENTGSDEARQAVKEIARIAKEALEEGCCDTAKEAIQRLEDLARDYSGSDVASLAVKAIAKIAETALRNGCCDTAKEAIQRLEDLARDYSGSDVASLAVKAIAKIAETALRNGCKETAEEAIKRLRELAEDYKGSEVAKLAEEAIERIEKVSRERGG

SEQ ID NO: 103 - protein, 174 aa, Artificial Sequence
  Synthetic peptide
  MISC_FEATURE (1)..(1): Optional residue
MSNDEKEKLKELLKRAEELAKSPDPEDLKEAVRLAEEVVRERPGSNLAKKALEIILRAAEELAKLPDPEALKEAVKAAEKVVREQPGSNLAKKALEIILRAAEELAKLPDPEALKEAVKAAEKVVREQPGSELAKKALEIIERAAEELKKSPDPEAQKEAKKAEQKVREERPGG

SEQ ID NO: 104 - protein, 174 aa, Artificial Sequence
  Synthetic peptide
  MISC_FEATURE (1)..(1): Optional residue
MTTEDERRELEKVARKAIEAAREGNTDEVREQLQRALEIARESGTTEAVKLALEVVARVAIEAARRGNTDAVREALEVALEIARESGTTEAVKLALEVVARVAIEAARRGNTDAVREALEVALEIARESGTEEAVRLALEVVKRVSDEAKKQGNEDAVKEAEEVRKKIEEESGG

SEQ ID NO: 105 - protein, 186 aa, Artificial Sequence
  Synthetic peptide
  MISC_FEATURE (1)..(1): Optional residue
MSSVAEEIEKRAKKISKELKKEGKNPEWIEELQRAADKLVEVARRATSSDALEIAKRAVKIAEELAKQGSNPKWIAELLKAAAKLVEVAARATSSDALEIAKRAVKIAEELAKQGSNPKWIAELLKAAAKLVEVAARATSPKALKQAKEAVKEAEELAKKGRNPKEIAEELKKRAKEVEKLARSTG

SEQ ID NO: 106 - protein, 190 aa, Artificial Sequence
  Synthetic peptide
  MISC_FEATURE (1)..(1): Optional residue
MSTEELKKVLERVRELSERAKESTDPEEALKIAKEVIELALKAVKEDPSTDALRAVLEAVRLASEVAKRVTDPDKALKIAKLVIELALEAVKEDPSTDALRAVLEAVRLASEVAKRVTDPDKALKIAKLVIELALEAVKEDPSEEAKRAVEEAKRLAEEVSKRVTDPELSEKIRQLVKELEEEAQKEDPG

SEQ ID NO: 107 - protein, 190 aa, Artificial Sequence
  Synthetic peptide
  MISC_FEATURE (1)..(1): Optional residue
MSTEELKKVLERVRELCERAKESTDPEEALKIAKEVIELALKAVKEDPSTDALRAVLEAVRCACEVAKRVTDPDKALKIAKLVIELALEAVKEDPSTDALRAVLEAVRCACEVAKRVTDPDKALKIAKLVIELALEAVKEDPSEEAKRAVEEAKRCAEEVSKRVTDPELSEKIRQLVKELEEEAQKEDPG

SEQ ID NO: 108 - protein, 194 aa, Artificial Sequence
  Synthetic peptide
  MISC_FEATURE (1)..(1): Optional residue
MKTEVEKKAKEVIKEAKELAKELDSEEAKKVVERIKEAAEAAKRAAEQGKTEVAKLALKVLEEAIELAKENRSEEALKVVLEIARAALAAAQAAEEGKTEVAKLALKVLEEAIELAKENRSEEALKVVLEIARAALAAAQAAEEGKSDEARDALRRLEEAIEEAKENRSKESLEKVREEAKEAEQQAEDAREGG

SEQ ID NO: 109 - protein, 174 aa, Artificial Sequence
  Synthetic peptide
  MISC_FEATURE (1)..(1): Optional residue
MDNDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIAKEAEKQGNVEVAVKAARVAVEAAKQAGDQDVLRKVSEQAERISKEAKKQGNSEVSEEARKVADEAKKQTGG

SEQ ID NO: 110 - protein, 230 aa, Artificial Sequence
  Synthetic peptide
  MISC_FEATURE (1)..(1): Optional residue
MDPEDELKRVEKLVKEAEELLRQAKEKGSEEDLEKALRTAEEAAREAKKVLEQAEKEGDPEVALRAVELVVRVAELLLRIAKESGSEEALERALRVAEEAARLAKRVLELAEKQGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREGG

SEQ ID NO: 111 - protein, 246 aa, Artificial Sequence
  Synthetic peptide
  MISC_FEATURE (1)..(1): Optional residue
MTPRERLEEAKERVEEIRELIDKARKLQEQGNKEEAEKVLREAREQIREVTRELEEIAKNSDTPELALRAAELLVRLIKLLIEIAKLLQEQGNKEEAEKVLREATELIKRVTELLEKIAKNSDTPELALRAAELLVRLIKLLIEIAKLLQEQGNKEEAEKVLREATELIKRVTELLEKIAKNSDTPELAKRAAELLKRLIELLKEIAKLLEEEGNEDEAEKVKEEAKELEERVRELEERIRKNSDG

SEQ ID NO: 112 - protein, 202 aa, Artificial Sequence
  Synthetic peptide
  MISC_FEATURE (1)..(1): Optional residue
MSTEEKIEEARQSIKEAERSLREGNPEKAREDVRRALELVRELEKLARKTGSTEVLIEAARLAIEVARVALKVGSPETAREAVRTALELVQELERQARKTGSTEVLIEAARLAIEVARVALKVGSPETAREAVRTALELVQELERQARKTGSDEVLKRAAELAKEVARVAKEVGSPETARQARETAERLREELRRNREKKGG

SEQ ID NO: 113 - protein, 202 aa, Artificial Sequence
  Synthetic peptide
  MISC_FEATURE (1)..(1): Optional residue
MDPEEILERAKESLERAREASERGDEEEFRKAAEKALELAKRLVEQAKKEGDPELVLEAAKVALRVAELAAKNGDKEVFKKAAESALEVAKRLVEVASKEGDPELVLEAAKVALRVAELAAKNGDKEVFKKAAESALEVAKRLVEVASKEGDPELVEEAAKVAEEVRKLAKKQGDEEVYEKARETAREVKEELKRVREEKGG

SEQ ID NO: 114 - protein, 150 aa, Artificial Sequence
  Synthetic peptide
  MISC_FEATURE (1)..(1): Optional residue
MDSTKEKARQLAEEAKETAEKVGDPELIKLAEQASQEGDSEKAKAILLAAEAARVAKEVGDPELIKLALEAARRGDSEKAKAILLAAEAARVAKEVGDPELIKLALEAARRGDSEKARAILEAAERAREAKERGDPEQIKKARELAKRGG

SEQ ID NO: 115 - protein, 222 aa, Artificial Sequence
  Synthetic peptide
  MISC_FEATURE (1)..(1): Optional residue
MNPELEEWIRRAKEVAKEVEKVAQRAEEEGNPDLRDSAKELRRAVEEAIEEAKKQGNPELVEWVARAAKVAAEVIKVAIQAEKEGNRDLFRAALELVRAVIEAIEEAVKQGNPELVEWVARAAKVAAEVIKVAIQAEKEGNRDLFRAALELVRAVIEAIEEAVKQGNPELVERVARLAKKAAELIKRAIRAEKEGNRDERREALERVREVIERIEELVRQGG

SEQ ID NO: 116 - protein, 206 aa, Artificial Sequence
  Synthetic peptide
  MISC_FEATURE (1)..(1): Optional residue
MNSDEEEAREWAERAEEAAKEALEQAKREGDEDARRVAEELEKQAEEARRKKDSEEAEAVYWAARAVLAALEALEQAKREGDEDARRVAEELLRQAEEAARKKNSEEAEAVYWAARAVLAALEALEQAKREGDEDARRVAEELLRQAEEAARKKNPEEARAVYEAARDVLEALQRLEEAKRRGDEEERREAEERLRQAEERARKKG

SEQ ID NO: 117 - protein, 206 aa, Artificial Sequence
  Synthetic peptide
  MISC_FEATURE (1)..(1): Optional residue
MNSDEEEAREWAERAEEAAKEALEQAKREGDEDARRCAEELEKQAEEARRKKDSEEAEAVYWAARAVLAALEALEQAKREGDEDARRCAEELLRQACEAARKKNSEEAEAVYWAARAVLAALEALEQAKREGDEDARRCAEELLRQACEAARKKNPEEARAVYEAARDVLEALQRLEEAKRRGDEEERREAEERLRQACERARKKG

SEQ ID NO: 118 - protein, 226 aa, Artificial Sequence
  Synthetic peptide
  MISC_FEATURE (1)..(1): Optional residue
MSSDEEEARELIERAKEAAERAQEAAERTGDPRVRELARELKRLAQEAAEEVKRDPSSSDVNEALKLIVEAIEAAVRALEAAERTGDPEVRELARELVRLAVEAAEEVQRNPSSSDVNEALKLIVEAIEAAVRALEAAERTGDPEVRELARELVRLAVEAAEEVQRNPSSEEVNEALKKIVKAIQEAVESLREAEESGDPEKREKARERVREAVERAEEVQRDPSG

SEQ ID NO: 119 - protein, 230 aa, Artificial Sequence
  Synthetic peptide
  MISC_FEATURE (1)..(1): Optional residue
MNSEELERESEEAERRLQEARKRSEEARERGDLKELAEALIEEARAVQELARVASERGNSEEAERASEKAQRVLEEARKVSEEAREQGDDEVLALALIAIALAVLALAEVASSRGNSEEAERASEKAQRVLEEARKVSEEAREQGDDEVLALALIAIALAVLALAEVASSRGNKEEAERAYEDARRVEEEARKVKESAEEQGDSEVKRLAEEAEQLAREARRHVQETRGG

SEQ ID NO: 120 - protein, 230 aa, Artificial Sequence
  Synthetic peptide
  MISC_FEATURE (1)..(1): Optional residue
MNSEELERESEEAERRLQEARKRSEEARERGDLKELAEALIEEARAVQELARVACERGNSEEAERASEKAQRVLEEARKVSEEAREQGDDEVLALALIAIALAVLALAEVACCRGNSEEAERASEKAQRVLEEARKVSEEAREQGDDEVLALALIAIALAVLALAEVACCRGNKEEAERAYEDARRVEEEARKVKESAEEQGDSEVKRLAEEAEQLAREARRHVQECRGG

SEQ ID NO: 121 - protein, 346 aa, Artificial Sequence
  Synthetic peptide
MSAEKLMLMAKLIIIVAENAKRKGDDTLIAIMAAKLAFEIVRIAAEEAGIDSSEVLELAIRLIKEVVENAQREGYDISIAALAAAMAFALVAIAAKRAGITSPEVLKLAIILIKLVVLAAQLSGYDIEEAAKKAAETFLRVAEEAREKGIDPREVIARSIADAAEEAATLAVRKGDEESLKSIVRLAATAAKTAKNPEVITKIVNLLLEIAERATDNELVNEIVKQLAEVAKEATDKDLVIHIVRILAELAKHSTDSELVNEIVKQLAEVAKRATDKELVIEIVRILAELAKESTDSRLVEEIVRQLKEVAERATDKELVEEIEKILEELKKESTDGWLEHHHHHH

SEQ ID NO: 122 - protein, 346 aa, Artificial Sequence; identical to SEQ ID NO: 121
  Synthetic peptide
  MISC_FEATURE (1)..(1): Optional residue
  MISC_FEATURE (337)..(346): Optional residues

SEQ ID NO: 123 - protein, 381 aa, Artificial Sequence
  Synthetic peptide
MHHHHHHHGGSGENLYFQGGSGWGNEEEEKLKELLERAKELAKSPDPEDLKEAVRLAEEVVRERPGSEAAKKALEIIQEAAELLKESPDPEAIIAAARALLKIAATTGDEEAAKEAIEAAEKAARLAEERGDDELVCEALALLIAARVLLLKQQGTSDEEVAETVARTISKLVKRLKKKGASEEVICECVARIVAEIVKALKRSGTSEEEIAEIVARVISEVIRTLEESGSSYEVICECVARIVAEIVEALKRSGTSAVEIAKIVARVISEVIRTLKESGSSYEVICECVARIVAEIVEALKRSGTSAAIIALIVALVISEVIRTLKESGSSFEVILECVIRIVLEIIEALKRSGTSEQDVMLIVMAVLLVVLATLQLSGS

SEQ ID NO: 124 - protein, 381 aa, Artificial Sequence; identical to SEQ ID NO: 123
  Synthetic peptide
  MISC_FEATURE (1)..(24): Optional residues

SEQ ID NO: 125 - protein, 405 aa, Artificial Sequence
  Synthetic peptide
MSAEKLMLMAKLIIIVAENAKRKGDDTLIAIMAAKLAFEIVRIAAEEAGIDSSEVLELAIRLIKEVVENAQREGYDISIAALAAAMAFALVAIAAKRAGITSSEVLELAIRLIKKVVENAQREGYDIEEAARAAAEAFERVAEAAKRAGITSSKAIKIAIELIEVVVRAASRNGHDISKAARKAAETIKTAADLAKKGNPDELAKHIAKTVEELKRNGVSEDEIARTVAAIIAFVIQALKSSGSSEDVIATIVARIVAEIVRALKRSGTSEDEIAEIVAKVISEVIRTLKESGSSHEVIAKIVARIVAEIVEALKDSGTSEEEIAKIVAHVISEVIRTLKESGSSEEVIHHIVKRIVHEIVKALKESGTSEDEIREIVKHVEHEVERTLHESGSSGWLEHHHHHH

SEQ ID NO: 126 - protein, 405 aa, Artificial Sequence; identical to SEQ ID NO: 125
  Synthetic peptide
  MISC_FEATURE (1)..(1): Optional residue
  MISC_FEATURE (396)..(405): Optional residues

SEQ ID NO: 127 - protein, 361 aa, Artificial Sequence
  Synthetic peptide
MHHHHHHHGGSGENLYFQGGSGWGSEEVNERVKQLAEKAKEATDKEEVIEIVKELAELAKQSTDPNLVAEVVRALTEVAKTSTDTELIREIIKVLLELASKLRDPQAVLEALQAVAELARELAEKTGDPIAKLCAIAVSLAAEAVKKAAELLKRHPDSQAAQDALKLAKQAAEAVLLACLLALEHPNAIIAILCIVAAIAAAIAASMAAALAQRHPDSQAARDAIKLASQAAEAVKLACELAQEHPNAKIAVLCILAAALAAIAAALAALLAQLHPDSQAARDAIKLASQAAEAVKLACELAQEHPNADIAEKCILLAILAALLAILAALLAMLHPDSQLARDLIDLASELAEEVKERCER

SEQ ID NO: 128 - protein, 361 aa, Artificial Sequence; identical to SEQ ID NO: 127
  Synthetic peptide
  MISC_FEATURE (1)..(24): Optional residues

SEQ ID NO: 129 - protein, 324 aa, Artificial Sequence
  Synthetic peptide
MHHHHHHHGGSGENLYFQGGSGWGNEEEEHLKELLKRAEELAKSPDPDDLREAVRLAEEVVRTRPGSELAKKALEIILRAAEELAKLPDPEALHEAVRAAEHVVRSQPGSEAAKEALRIIQEAAELLKESPDPTAIIRAARALLKIARTTGDEEAAKEAIEAAKKAADLARERGDDELVCEALALLVAAQVELLKQQGTSAVEIAKIVARVISEVIRTLKEKGSSYEVICECVARIVAEIVEALKRSGTSAAIIALIVALVISEVIRTLKESGSSFEVILECVIRIVLEIIEALKRSGTSEQDVMLIVMAVLLVVLATLQLSGS

SEQ ID NO: 130 - protein, 324 aa, Artificial Sequence; identical to SEQ ID NO: 129
  Synthetic peptide
  MISC_FEATURE (1)..(24): Optional residues

SEQ ID NO: 131 - protein, 573 aa, Artificial Sequence
  Synthetic peptide
MHHHHHHHGGSGENLYFQGGSGWGSELGKRLIEAAENGNKDRVKDLIENGADVNASDSDGRTPLHHAAENGHKEVVKLLISKGADVNAKDSDGRTPLHHAAENGHKEVVKLLISKGADVNAKDSDGRTPLHHAAENGHKEVVKLLISKGADVNAKADRGMTPLHFAAWRGHKEVVKLLISKGADLNTSAKDGATPVLLALRRGDEEVVRLLKEEAKKRGDEFLARCAEAAELAIEALKLAEELLRRYPNDEAARLAHHLAKLALEAVELACILASEHPNADIAKLCIKAASEAAEAASKAAELAQRHPDSQAARDAIKLASQAAEAVKLACELAQEHPNADIAKLCIIAASLAAEAASKAAELAQRHPDSQAARDAIKLASQAAEAVKLACELAQEHPNAIIAILCIVAAIAAAIAASMAAALAQRHPDSQAARDAIKLASQAAEAVKLACELAQEHPNAKIAVLCILAAALAAIAAALAALLAQLHPDSQAARDAIKLASQAAEAVKLACELAQEHPNADIAEKCILLAILAALLAILAALLAMLHPDSQLARDLIDLASELAEEVKERCER

SEQ ID NO: 132 - protein, 573 aa, Artificial Sequence; identical to SEQ ID NO: 131
  Synthetic peptide
  MISC_FEATURE (1)..(24): Optional residues

SEQ ID NO: 133 - protein, 506 aa, Artificial Sequence
  Synthetic peptide
MIEEVVAEMIDILAESSKKSIEELARAADNKTTEKAVAEAIEEIARLATAAIQLIEALAKNLASEEFMARAISAIAELAKKAIEAIYRLADNHTTDTFMARAIAAIANLAVTAILAIAALASNHTTEEFMARAISAIAELAKKAIEAIYRLADNHTTDKFMAAAIEAIALLATLAILAIALLASNHTTEEFMAKAISAIAELAKKAIEAIYRLADNHTNEELIRHAIEIIREIAEIAARAIIEIAKRLKSEEYALHALRAVLEIIEHALERIARKADKEEKKALELLIEVAREIYRLAEEAAKRAKDEEEAAKIAVIAAEAILELLRAQRKVTDNEVIEKLLEVVKEIIRLAEEAMKKMTDEEEAAKIAKEALEAIKMLARAVEEVTDNEVIEKLLEVVKEIIRLAEEAMKKMTDEEEAAKIAKEALEAIKMLARAVEEVTDKERIEQLLREVKEEIRRAEEESRKETDDEEAAKRAREALRRIRERAREVEEDKSGWLEHHHHHH

SEQ ID NO: 134 - protein, 506 aa, Artificial Sequence; identical to SEQ ID NO: 133
  Synthetic peptide
  MISC_FEATURE (1)..(1): Optional residue
  MISC_FEATURE (497)..(506): Optional residues

SEQ ID NO: 135 - protein, 463 aa, Artificial Sequence
  Synthetic peptide
MIEEVVAEMIDILAESSKKSIEELARAADNKTTEKAVAEAIEEIARLATAAIQLIEALAKNLASEEFMARAISAIAELAKKAIEAIYRLADNHTTDTFMARAIAAIANLAVTAILAIAALASNHTTEEFMARAISAIAELAKKAIEAIYRLADNHTTDKFMAAAIEAIALLATLAILAIALLASNHTTEEFMAKAISAIAELAKKAIEAIYRLADNHTNEEAIHEAAEAILRIAEEAIRAIEELVRRSKSEEIEERAKKLIEEIARKAIEAALRLGSEEIAARVAYILIEIIIKRHPGDKEEAAEIARKIIEQIIRTLPGGCDCVAKAASSIIRAVIEKNPNYSEVVADVAAAIVKAIIEGNPNGCDCVAKAASSIIRAVIEKNPNYSEVVADVAAAIVKAIIEGNPNGRDCVRKAASSIIRAVQEKNPNYSEVVEDVKRAIEKAIKEGNPNGGWLEHHHHHH

SEQ ID NO: 136 - protein, 463 aa, Artificial Sequence; identical to SEQ ID NO: 135
  Synthetic peptide
  MISC_FEATURE (1)..(1): Optional residue
  MISC_FEATURE (454)..(463): Optional residues

SEQ ID NO: 137 - protein, 463 aa, Artificial Sequence; identical to SEQ ID NO: 135
  Synthetic peptide

SEQ ID NO: 138 - protein, 463 aa, Artificial Sequence; identical to SEQ ID NO: 135
  Synthetic peptide
  MISC_FEATURE (1)..(1): Optional residue
  MISC_FEATURE (454)..(463): Optional residues

SEQ ID NO: 139 - protein, 991 aa, Artificial Sequence
  Synthetic peptide
HHHHHHHGGSGENLYFQGGSGWGSEEVNKKVEDLAREAQKATDKETVIRIVETLAELAKKSTDKDLVNEIVRQLAEVAKQATDKELVIRIVEILAELAKTSTDSELVNEIVKQLAEVAKRATDPELVIRIVEILAELAKTSTDSELVNEIVKQLAEVAKRATDPDLVIYIVTILAELAKTSTDKDLVNEIVKQLAEVAKRATDKDLVIYIVTILAELAKTSTDSKLVEEIVKQLAEVAKRATDKELVIYIVTILAELAKTSTDSELVNEIVKQLAEVAKRATDKELVIYIVHILARLAQTSTDSELVNEIVKQLAEVAKRATDKELVIYIVEILARLADTSTDQELVRRIVQQLAQVAKRATDNELVIYIVEILAELAKRSTDPKVVAEILQALAEVAQQSTDPELARKIIEVIAELAKDQGDSALLQAAEAAKKAANKGNERLLLAVLQALLVAVEVLIVAEEARENGNKELADAATRLIKAVARAITEAVDQGNPELVKWVAEAAKVAADVIRVAIQANREGNSQLFKAALRLVEAVIEAIKEAVDQGNPELVHWVARAAKVAADVIRVAIQAKKEGNEELFQAALRLVQAVIEAIKEAVKQGNPELVEWVARAAKVAADVIRVAIQAKREGNRELFEAALRLVQAVIEAIKEAVKQGNPELVEWVARAAKVAAEVIKVAIQAKREGNEELFQAALRLVQAVIEAIKEAVKQGNPELVEWVARAATVAAEVIKVAIQAKKEGNPDLFRAALRLVDAVIEAIKRAVKQGNPELVEWVARAAHVAARVIEVAIQAKREGNPELFKAALRLVDAVIEAIKRAVRQGNPELVEWVARAAKVAAEVIKVAIQAKKEGNRELFEAALRLVDAVIEAIKRAVRQGNPELVEWVARAAHVAARVIEVAIQAKKEGNPDLFRAALRLVQAVIEAIKEAVRQGNPELVERVARLATHAAELIKEAIKAKREGNDDKRRRALETVQKVIEDIKELVRQGN

SEQ ID NO: 140 - protein, 991 aa, Artificial Sequence; identical to SEQ ID NO: 139
  Synthetic peptide
  MISC_FEATURE (1)..(23): Optional residues

SEQ ID NO: 141 - protein, 869 aa, Artificial Sequence
  Synthetic peptide
HHHHHHHGGSGENLYFQGGSGWGDSEEVNDKVRRLAKKAKDATDKETVIRIVHTLARLAEKSTDKDLVNEIVKQLAEVAKRATDKELVIRIVEILARLAERSTDSELVNEIVKQLAEVAKRATDQELVIRIVEILAELAKRSTDKDLVNEIVKQLAEVAKRATDQDLVIRIVEILAELAKTSTDKDLVNEIVKQLAEVAKRATDPDLVIRIVEILAELAKTSTDSKLVNDIVKQLAEVAKRATDKDLVIRIVHILHRLAQTSTDDELVNEIVRQLAEVARRATDRELVIHIVTILAKLAEESTDEKAIQEIAERLATVAKESQDEELILTIILVLLRLLSTSTDPEALEQIARAVLELARQNGDEKLAELAEEALRAVQTAKEAKEKGDEDLAQAALLIALAAAAAAAALIAARQTGDPRVRRLAEELKRLAQEAAERVKRDPSSEETLRALTIIIIAIEVAVIALEVARKQGNPNVKRRASELVEQAVRAAQEVNDDPTDEAVYNAVHTLARAALQAVKDGPDTQEVVKKALEVVAKLAIIAARQGSTDAVRDALQVALEIARTAGNQEAVKLALEVVAQVAIEAAKTGNTDAVREALRVALQIARTSGTEEAVKLALEVVARVAIEAARRGNTDAVRDALEVALQIARTSGTEEAVKLALEVVARVAIEAARRGNTEAVREALEVALKIAKTSGTQEAVKLALEVVARVAIEAARRGNTEAVRDALRVALKIAKTSGTEEAVKLALEVVARVAIEAARRGNTDAVRDALQVALEIAKTSGTEEAVKLALEVVARVAIEAARRGNTDAVREALEVALQIARTSGTDEAVKLALEVVKRVSDEARRRGNEEAVKEAEEVRERIERTQGT

SEQ ID NO: 142 - protein, 869 aa, Artificial Sequence; identical to SEQ ID NO: 141
  Synthetic peptide
  MISC_FEATURE (1)..(23): Optional residues

SEQ ID NO: 143 - protein, 234 aa, Artificial Sequence
  Synthetic peptide
  MISC_FEATURE (1)..(1): Optional residue
MNDEEVQEAVERAEELREEAEELIKKARKTGDPELLRKALEALEEAVRAVEEAIKRNPDNDEAVETAVRLARELKKVAEELQERAKKTGDPELLKLALRALEVAVRAVELAIKSNPDNDEAVETAVRLARELKKVAEELQERAKKTGDPELLKLALRALEVAVRAVELAIKSNPDNEEAVETAKRLAEELRKVAELLEERAKETGDPELQELAKRAKEVADRARELAKKSNPNG

SEQ ID NO: 144 - protein, 10 aa, Artificial Sequence
  Synthetic peptide
GWLEHHHHHH

SEQ ID NO: 145 - protein, 10 aa, Artificial Sequence
  Synthetic peptide
HHHHHHHGGS

SEQ ID NO: 146 - protein, 8 aa, Artificial Sequence
  Synthetic peptide
GENLYFQG

SEQ ID NO: 147 - protein, 5 aa, Artificial Sequence
  Synthetic peptide
GSGWG